(19) United States
(12) Patent Application Publication
(10) Pub. No.: US 2021/0361634 A1
(43) Pub. Date: Nov. 25, 2021

(54) THERAPEUTIC COMPOUNDS AND COMPOSITIONS

(71) Applicant: eXIthera Pharmaceuticals, Inc., Westborough, MA (US)

(72) Inventors: Neil J. Hayward, Westborough, MA (US); Bertrand L. Chenard, Waterford, CT (US); Yuelian Xu, East Haven, CT (US); Philipp Erik Schneggenburger, Arlington, MA (US); Matteo Placido Placidi, Arlington, MA (US); Wendy Mercer Geil, Concord, MA (US); Gonto Johns., III, Chicago, IL (US)

(21) Appl. No.: 17/388,859

(22) Filed: Jul. 29, 2021

Related U.S. Application Data
(63) Continuation of application No. PCT/US2020/015002, filed on Jan. 24, 2020.
(60) Provisional application No. 62/798,012, filed on Jan. 29, 2019.

(51) Int. Cl. A61K 31/4427; A61K 47/40; A61K 47/26; A61K 47/02; A61K 9/00; A61P 7/02

(52) U.S. Cl. CPC A61K 31/4427; A61K 47/40; A61P 7/02; A61K 47/02; A61K 9/0019; A61K 47/26

(57) ABSTRACT

Provided herein are pharmaceutical compositions comprising compounds that inhibit Factor XIa or kallikrein and methods of use thereof.

### CROSS-REFERENCE TO RELATED APPLICATIONS

This application is a continuation of International Application No. PCT/US2020/015002, filed Jan. 24, 2020, which claims priority to U.S. Ser. No. 62/798,012 filed Jan. 29, 2019, which is incorporated herein by reference in its entirety.

### BACKGROUND OF THE INVENTION

Blood coagulation is the first line of defense against blood loss following injury. The blood coagulation “cascade” involves a number of circulating serine protease zymogens, regulatory cofactors and inhibitors. Each enzyme, once generated from its zymogen, specifically cleaves the next zymogen in the cascade to produce an active protease. This process is repeated until finally thrombin cleaves the fibrinopeptides from fibrinogen to produce fibrin that polymerizes to form a blood clot. Although efficient clotting limits the loss of blood at a site of trauma, it also poses the risk of systemic coagulation resulting in massive thrombosis. Under normal circumstances, hemostasis maintains a balance between clot formation (coagulation) and clot dissolution (fibrinolysis). However, in certain disease states such as acute myocardial infarction and unstable angina, the rupture of an established atherosclerotic plaque results in abnormal thrombus formation in the coronary arterial vasculature.

Diseases that stem from blood coagulation, such as myocardial infarction, unstable angina, atrial fibrillation, stroke, pulmonary embolism, and deep vein thrombosis, are among the leading causes of death in developed countries. Current anticoagulant therapies, such as injectable unfractionated and low molecular weight (LMW) heparin and orally administered warfarin (coumadin), carry the risk of bleeding episodes and display patient-to-patient variability that results in the need for close monitoring and titration of therapeutic doses. Consequently, there is a large medical need for novel anticoagulation drugs that lack some or all of the side effects of currently available drugs.

Factor XIa is an attractive therapeutic target involved in the pathway associated with these diseases. Increased levels of Factor XIa or Factor XIa activity have been observed in several thromboembolic disorders, including venous thrombosis (Meijers et al., N. Engl. J. Med. 342:696, 2000), acute myocardial infarction (Minnema et al., Arterioscler Thromb Vasc Biol 20:2489, 2000), acute coronary syndrome (Butenas et al., Thromb Haemost 99:142, 2008), coronary artery disease (Butenas et al., Thromb Haemost 99:142, 2008), chronic obstructive pulmonary disease (Jankowski et al., Thromb Res 127:242, 2011), aortic stenosis (Blood Coagul Fibrinolysis, 22:473, 2011), acute cerebrovascular ischemia (Undas et al., Eur J Clin Invest, 42:123, 2012), and systolic heart failure due to ischemic cardiomyopathy (Zabcyk et al., Pol Arch Med Wewn. 120:334, 2010). Patients that lack Factor XI because of a genetic Factor XI deficiency exhibit few, if any, ischemic strokes (Salomon et al., Blood, 111:4113, 2008). At the same time, loss of Factor XIa activity, which leaves one of the pathways that initiate coagulation intact, does not disrupt hemostasis. In humans, Factor XI deficiency can result in a mild-to-moderate bleeding disorder, especially in tissues with high levels of local fibrinolytic activity, such as the urinary tract, nose, oral cavity, and tonsils. Moreover, hemostasis is nearly normal in Factor XI-deficient mice (Gailani, Blood Coagul Fibrinolysis, 8:134, 1997). Furthermore, inhibition of Factor XI has also been found to attenuate arterial hypertension and other diseases and dysfunctions, including vascular inflammation (Kossmann et al. Sci. Transl. Med. 9, eaah4923 (2017)).

Consequently, compounds that inhibit Factor XIa have the potential to prevent or treat a wide range of disorders while avoiding the side effects and therapeutic challenges that plague drugs that inhibit other components of the coagulation pathway. Moreover, due to the limited efficacy and adverse side effects of some current therapeutics for the inhibition of undesirable thrombosis (e.g., deep vein thrombosis, hepatic vein thrombosis, and stroke), improved compounds and methods (e.g., those associated with Factor XIa) are needed for preventing or treating undesirable thrombosis.

Another therapeutic target is the enzyme kallikrein. Human plasma kallikrein is a serine protease that may be responsible for activating several downstream factors (e.g., bradykinin and plasmin) that are critical for coagulation and control of e.g., blood pressure, inflammation, and pain. Kallikreins are expressed e.g., in the prostate, epidermis, and the central nervous system (CNS) and may participate in e.g., the regulation of semen liquefaction, cleavage of cellular adhesion proteins, and neuronal plasticity in the CNS. Moreover, kallikreins may be involved in tumorigenesis and the development of cancer and angioedema, e.g., hereditary angioedema. Overactivation of the kallikrein-kinin pathway can result in a number of disorders, including angioedema, e.g., hereditary angioedema (Schneider et al., J. Allergy Clin. Immunol. 120:2, 416, 2007). To date, there are limited treatment options for HAE (e.g., WO2003/076458).

Pharmaceutical compositions comprising a therapeutic agent, e.g., compounds that inhibit Factor Xia or kallikrein described herein, enable administration to a human subject in need by various modes of administration (e.g., parenteral (e.g., intravenous, intramuscular, subcutaneous) delivery). Particularly for intravenous or subcutaneous administration, compositions are generally pH stable or chemically stable, preferably for an extended period of time.

### SUMMARY OF THE INVENTION

The present invention relates, in part, to pharmaceutical compositions comprising a compound of Formula (I-A):

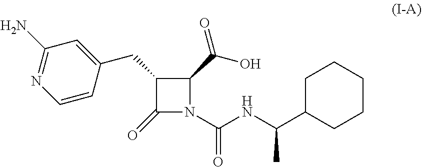 [SMILES: C[C@@H](NC(=O)N1C(=O)[C@H](Cc2ccnc(N)c2)[C@H]1C(=O)O)C1CCCCC1]

also referred to herein as “Compound 1,” or a pharmaceutically acceptable salt thereof.

Thus, in an aspect, provided herein is an aqueous pharmaceutical composition comprising a compound of Formula (I-A)

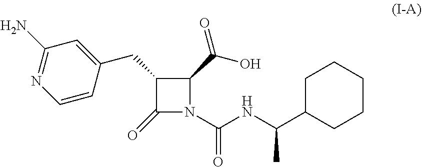 [SMILES: C[C@@H](NC(=O)N1C(=O)[C@H](Cc2ccnc(N)c2)[C@H]1C(=O)O)C1CCCCC1]

or a pharmaceutically acceptable salt thereof, a cyclodextrin, and an excipient.

In some embodiments, the pharmaceutical composition comprises the compound of Formula (I-A), the cyclodextrin, and the excipient. In some embodiments, the cyclodextrin is selected from the group consisting of alkyl cyclodextrin, hydroxyalkyl cyclodextrin, carboxyalkyl cyclodextrin, and sulfoalkyl ether cyclodextrin. In some embodiments, the cyclodextrin is hydroxypropyl β-cyclodextrin. In some embodiments, the cyclodextrin is sulfobutyl ether β-cyclodextrin.

In some embodiments, the excipient is a sugar (e.g., a saccharide (e.g., monosaccharide, disaccharide, or polysaccharide)) or a sugar alcohol. In some embodiments, the excipient is sucrose, lactose, trehalose, dextran, erythritol, arabitol, xylitol, sorbitol, or mannitol, or a combination thereof. In some embodiments, the excipient is mannitol. In some embodiments, the excipient is lactose.

In some embodiments, the pharmaceutical composition further comprises a buffer. In some embodiments, the buffer is a monoprotic acid or a polyprotic acid or a combination thereof. In some embodiments, the buffer is a solution of one or more substances. In some embodiments, the buffer is a solution of a salt of a weak acid and a weak base. In some embodiments, the buffer is a solution of a salt of the weak acid with a strong base. In some embodiments, the buffer is selected from the group consisting of a maleate buffer, a citrate buffer, and a phosphate buffer. In some embodiments, the buffer is a phosphate buffer. In some embodiments, the phosphate buffer is a solution of monosodium phosphate, disodium phosphate, trisodium phosphate, or a combination thereof.

In some embodiments, the pharmaceutical compositions described herein further comprises a solubilizing agent. In some embodiments, the solubilizing agent is a polyoxyethylene sorbitan ester (e.g, TWEEN® 20) or a polyethylene glycol (e.g., PEG400).

In some embodiments, the pH is from about 2 to about 8. In some embodiments, the pH is about 6.8.

In some embodiments, the concentration of the compound of Formula (I-A) is from about 0.1 mg/mL to about 100 mg/mL. For example, the concentration of the compound of Formula (I-A) may be about 10 mg/mL.

In some embodiments, the concentration of the buffer is from about 1 mM to about 500 mM. For example, the concentration of the buffer may be about 10 mM. In some embodiments, the buffer is phosphate buffer.

In some embodiments, the cyclodextrin is in an amount of from about 0.1% to about 10% (e.g., about 0.5% to about 6% (e.g., about 0.7% to about 5.6% (e.g., about 2.1% to about 5%))) by weight relative to weight of the compound of Formula (I-A). For example, the cyclodextrin is in an amount of about 3.5% by weight relative to weight of the compound of Formula (I-A). As another example, the cyclodextrin is in an amount of about 5% by weight relative to weight of the compound of Formula (I-A). In some embodiments, the cyclodextrin is hydroxypropyl β-cyclodextrin.

In some embodiments, the excipient is in an amount of from about 0.1% to about 10% by weight relative to weight of the compound of Formula (I-A). For example, the excipient is in an amount of about 3% by weight relative to weight of the compound of Formula (I-A). As another example, the excipient is in an amount of about 5% by weight relative to weight of the compound of Formula (I-A). In some embodiments, the excipient is mannitol. In other embodiments, the excipient is lactose.

In another aspect, provided herein is pharmaceutical composition comprising particles, wherein the particles comprise a compound of Formula (I-A)

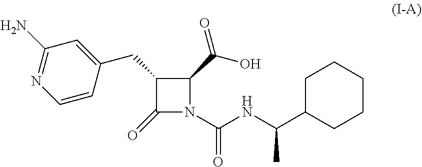 [SMILES: C[C@@H](NC(=O)N1C(=O)[C@H](Cc2ccnc(N)c2)[C@H]1C(=O)O)C1CCCCC1]

or a pharmaceutically acceptable salt thereof, a cyclodextrin, and a bulking agent.

In some embodiments, the cyclodextrin is selected from the group consisting of alkyl cyclodextrin, hydroxyalkyl cyclodextrin, carboxyalkyl cyclodextrin, and sulfoalkyl ether cyclodextrin. In some embodiments, the cyclodextrin is hydroxypropyl β-cyclodextrin. In some embodiments, the cyclodextrin is sulfobutyl ether β-cyclodextrin.

In some embodiments, the bulking agent is a sugar (e.g., a saccharide (e.g., monosaccharide, disaccharide, or polysaccharide)) or a sugar alcohol. In some embodiments, the bulking agent is sucrose, lactose, trehalose, dextran, erythritol, arabitol, xylitol, sorbitol, or mannitol, or a combination thereof. In some embodiments, the bulking agent is mannitol. In some embodiments, the bulking agent is lactose.

In some embodiments, the bulking agent is a lyoprotectant.

In some embodiments, the concentration of the compound of Formula (I-A) is from about 0.1 to about 10% by weight of the composition. For example, the concentration of the compound of Formula (I-A) is about 1% by weight of the composition. As another example, the concentration of the compound of Formula (I-A) is about 0.3% by weight of the composition.

In some embodiments, the cyclodextrin is in an amount of from about 0.1% to about 10% (e.g., about 0.5% to about 6% (e.g., about 0.7% to about 5.6% (e.g., about 2.1% to about 5%))) by weight relative to weight of the compound of Formula (I-A). For example, the cyclodextrin is in an amount of about 3.5% by weight relative to weight of the compound of Formula (I-A). As another example, the cyclodextrin is in an amount of about 5% by weight relative to weight of the compound of Formula (I-A). In some embodiments, the cyclodextrin is hydroxypropyl β-cyclodextrin.

In some embodiments, the bulking agent is in an amount of from about 0.1% to about 10% by weight relative to weight of the compound of Formula (I-A). For example, the bulking agent is in an amount of about 3% by weight relative to weight of the compound of Formula (I-A). As another example, the bulking agent is in an amount of about 5% by weight relative to weight of the compound of Formula (I-A). In some embodiments, the bulking agent is mannitol. In other embodiments, the bulking agent is lactose.

In another aspect, provided herein is a process for preparing an aqueous pharmaceutical composition from the pharmaceutical composition comprising particles, wherein the particles comprise a compound of Formula (I-A) or a pharmaceutically acceptable salt thereof, a cyclodextrin, and a bulking agent, the process comprising reconstituting the pharmaceutical composition into an aqueous medium, thereby forming the aqueous composition.

In some embodiments, the aqueous medium is deionized water. In some embodiments, the aqueous medium comprises sodium chloride. In some embodiments, the aqueous medium comprises about 5% dextrose. In some embodiments, composition is prepared to be suitable for parenteral administration to a subject in need thereof. For example, the composition is prepared to be suitable for intramuscular, subcutaneous or intravenous administration to a subject in need thereof.

The compositions described herein can be useful in the treatment, prophylaxis, or reduction in the risk of a disorder described herein. In some embodiments, the methods described herein can include those in which a subject's blood is in contact with an artificial surface.

Thus, in one aspect, provided herein is a method of treating a thromboembolic disorder in a subject in need thereof, the method comprising administering to the subject an effective amount of a pharmaceutical composition described herein, wherein the blood of the subject is contacted with an artificial surface.

In another aspect, provided herein is a method of reducing the risk of a thromboembolic disorder in a subject in need thereof, the method comprising administering to the subject an effective amount of a pharmaceutical composition described herein, wherein the blood of the subject is contacted with an artificial surface.

Also provided herein is a method of prophylaxis of a thromboembolic disorder in a subject in need thereof, the method comprising administering to the subject an effective amount of a pharmaceutical composition described herein, wherein the blood of the subject is contacted with an artificial surface.

In some embodiments of the methods described herein, the artificial surface is in contact with blood in the subject's circulatory system.

In some embodiments, the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus.

In some embodiments, the artificial surface causes or is associated with the thromboembolic disorder.

In some embodiments, the thromboembolic disorder is a venous thromboembolism, deep vein thrombosis, or pulmonary embolism.

In some embodiments, the thromboembolic disorder is a blood clot.

In some embodiments, the methods described herein further comprise conditioning the artificial surface with a separate dose of a pharmaceutical composition described herein prior to contacting the artificial surface with blood in the circulatory system of the subject.

In some embodiments, the methods described herein further comprise conditioning the artificial surface with a separate dose of a pharmaceutical composition described herein prior to or during administration of the pharmaceutical composition to the subject.

In some embodiments, the methods described herein further comprise conditioning the artificial surface with a separate dose of a pharmaceutical composition described herein prior to and during administration of the pharmaceutical composition to the subject.

In some embodiments of the methods described herein, the artificial surface is a cardiopulmonary bypass circuit.

In some embodiments of the methods described herein, the artificial surface is an extracorporeal membrane oxygenation (ECMO) apparatus. In some embodiments, the ECMO apparatus is venovenous ECMO apparatus or venoarterial ECMO apparatus.

In another aspect, disclosed herein is a method of preventing or reducing a risk of a thromboembolic disorder in a subject during or after a medical procedure, comprising:

(i) administering to the subject an effective amount of a pharmaceutical composition described herein, before, during, or after the medical procedure; and

(ii) contacting blood of the subject with an artificial surface;

thereby preventing or reducing the risk of the thromboembolic disorder during or after the medical procedure.

In some embodiments, the artificial surface is conditioned with a pharmaceutical composition described herein prior to administration of the pharmaceutical composition to the subject prior to, during, or after the medical procedure.

In some embodiments, the pharmaceutical composition for conditioning the artificial surface further comprises a solution, wherein the solution is selected from the group consisting of a saline solution, Ringer's solution, and blood.

In some embodiments, the thromboembolic disorder is a blood clot.

In some embodiments, the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises a cardiopulmonary bypass.

In some embodiments, the medical procedure comprises an oxygenation and pumping of blood via extracorporeal membrane oxygenation (ECMO). In some embodiments, the ECMO is venovenous ECMO or venoarterial ECMO.

In some embodiments of the methods described herein, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

In another aspect, provided herein is a method of treating the blood of a subject in need thereof, the method comprising administering to the subject an effective amount of a pharmaceutical composition described herein.

In some embodiments of the methods described herein, the pharmaceutical composition is administered to the subject intravenously. In other embodiments of the methods described herein, the pharmaceutical composition is administered to the subject subcutaneously. In some embodiments, the pharmaceutical composition is administered to the subject as a continuous intravenous infusion. In some embodiments, the pharmaceutical composition is administered to the subject as a bolus.

In some embodiments, the subject is a human. In some embodiments, the subject has an elevated risk of a thromboembolic disorder. In some embodiments, the thromboembolic disorder is a result of a complication in surgery. In some embodiments, the subject is sensitive to or has developed sensitivity to heparin. In some embodiments, the subject is resistant to or has developed resistance to heparin.

In another aspect, the present invention is also directed to a method of reducing the risk of stroke (e.g., ischemia, e.g., a transient ischemic event, large vessel acute ischemic stroke) in a subject that has suffered an ischemic event (e.g., a transient ischemic event), comprising administering to the subject an effective amount of a composition described herein (e.g., a composition comprising Compound 1 or a pharmaceutically acceptable salt thereof). In some embodiments, the administering reduces the risk of stroke (e.g., large vessel acute ischemic stroke) in a subject as compared to a subject who is not administered with the composition. In some embodiments, the administering reduces the risk of atrial fibrillation in a subject as compared to a subject who is not administered with the composition.

In one aspect, the present invention is directed to a method of reducing non-central nervous system systemic embolism (e.g., ischemia, e.g., a transient ischemic event) in a subject that has suffered an ischemic event (e.g., a transient ischemic event), comprising administering to the subject an effective amount of a composition described herein (e.g., a composition comprising Compound 1 or a pharmaceutically acceptable salt thereof). In some embodiments, the administering reduces non-central nervous system systemic embolism in a subject as compared to a subject who is not administered with the composition.

In one aspect, the present invention is directed to a method of treating deep vein thrombosis comprising administering to the subject that has suffered an ischemic event (e.g., a transient ischemic event), an effective amount of a composition described herein (e.g., a composition comprising Compound 1 or a pharmaceutically acceptable salt thereof).

In one aspect, the present invention is directed to a method of prophylaxis of deep vein thrombosis comprising administering to the subject that has suffered a deep vein thrombosis (e.g., a subject that has been previously treated for a deep vein thrombosis), an effective amount of a composition described herein (e.g., a composition comprising Compound 1 or a pharmaceutically acceptable salt thereof).

In one aspect, the present invention is directed to a method of reducing the risk of recurrence of deep vein thrombosis comprising administering to the subject that has suffered a deep vein thrombosis (e.g., a subject that has been previously treated for a deep vein thrombosis), an effective amount of a composition described herein (e.g., a composition comprising Compound 1 or a pharmaceutically acceptable salt thereof). In some embodiments, the administering reduces the risk of recurrence of deep vein thrombosis in a subject as compared to a subject who is not administered with the composition.

In one aspect, the present invention is directed to a method of prophylaxis of venous thromboembolism, e.g., deep vein thrombosis or pulmonary embolism in a subject, comprising administering to the subject an effective amount of a composition described herein (e.g., a composition comprising Compound 1 or a pharmaceutically acceptable salt thereof). In some embodiments, the subject is undergoing surgery. In some embodiments, the subject is administered the composition described herein before, during, or after surgery. In some embodiments, the subject is undergoing knee or hip replacement surgery. In some embodiments, the subject is undergoing orthopedic surgery. In some embodiments, the subject is undergoing lung surgery. In some embodiments, the subject is being treated for cancer, e.g., by surgery. In some embodiments, the subject is suffering from a chronic medical condition. In some embodiments, the venous thromboembolism is associated with cancer. In some embodiments, Compound 1, or a pharmaceutically acceptable salt thereof, in the composition described herein is a primary agent in prophylaxis of the deep vein thrombosis or venous thromboembolism. In some embodiments, Compound 1, or a pharmaceutically acceptable salt thereof, in the composition described herein is used as an extended therapy.

In one aspect, the present invention is directed to a method of reducing the risk of venous thromboembolism, e.g., deep vein thrombosis or pulmonary embolism, in a subject, comprising administering to the subject an effective amount of a composition described herein (e.g., a composition comprising Compound 1 or a pharmaceutically acceptable salt thereof). In some embodiments, the subject is undergoing surgery. In some embodiments, the subject is administered the composition described herein after surgery. In some embodiments, the subject is undergoing knee or hip replacement surgery. In some embodiments, the subject is undergoing orthopedic surgery. In some embodiments, the subject is undergoing lung surgery. In some embodiments, the subject is being treated for cancer, e.g., by surgery. In some embodiments, the subject is suffering from a chronic medical condition. In some embodiments, the thromboembolic disorder is associated with cancer. In some embodiments, Compound 1 or a pharmaceutically acceptable salt thereof in the composition described herein is a primary agent in reducing the risk of the thromboembolic disorder. In some embodiments, Compound 1 or a pharmaceutically acceptable salt thereof in the composition described herein is used as an extended therapy.

In one aspect, the present invention is directed to a method of reducing the risk of stroke (e.g., large vessel acute ischemic stroke) or systemic embolism in a subject in need thereof, comprising administering to the subject an effective amount of a composition described herein, e.g., a composition comprising Compound 1 or a pharmaceutically acceptable salt thereof. In some embodiments, the subject is suffering from atrial fibrillation (e.g., non-valvular atrial fibrillation). In some embodiments, the subject is suffering from a renal disorder (e.g., end-stage renal disease).

In one aspect, the present invention is directed to a method of prophylaxis of stroke (e.g., large vessel acute ischemic stroke) or systemic embolism in a subject in need thereof, comprising administering to the subject an effective amount of a composition described herein, e.g., a composition comprising Compound 1 or a pharmaceutically acceptable salt thereof. In some embodiments, the subject is suffering from atrial fibrillation (e.g., non-valvular atrial fibrillation). In some embodiments, the subject is suffering from a renal disorder (e.g., end-stage renal disease).

In one aspect, the present invention is directed to a method of reducing the risk of recurrence of pulmonary embolism (e.g., symptomatic pulmonary embolism) comprising administering to the subject that has suffered a pulmonary embolism (e.g., a subject that has been previously treated for a pulmonary embolism), an effective amount of a composition described herein (e.g., a composition comprising Compound 1 or a pharmaceutically acceptable salt thereof). In some embodiments, the administering reduces the risk of recurrence of pulmonary embolism in a subject as compared to a subject who is not administered with the composition.

In one aspect, the present invention is directed to a method of prophylaxis of pulmonary embolism in a subject that has suffered a pulmonary embolism (e.g., a subject that has been previously treated for a pulmonary embolism), comprising administering to the subject an effective amount of a composition described herein (e.g., a composition comprising Compound 1 or a pharmaceutically acceptable salt thereof).

In one aspect, the present invention is directed to a method of reducing the risk of recurrence of pulmonary embolism (e.g., symptomatic pulmonary embolism) comprising administering to the subject that has suffered a deep vein thrombosis (e.g., a subject that has been previously treated for a deep vein thrombosis), an effective amount of a composition described herein (e.g., a composition comprising Compound 1 or a pharmaceutically acceptable salt thereof). In some embodiments, the administering reduces the risk of recurrence of pulmonary embolism in a subject as compared to a subject who is not administered with the composition.

In one aspect, the present invention is directed to a method of prophylaxis of pulmonary embolism in a subject that has suffered a deep vein thrombosis (e.g., a subject that has been previously treated for a deep vein thrombosis), comprising administering to the subject a composition described herein (e.g., a composition comprising Compound 1 or a pharmaceutically acceptable salt thereof).

In one aspect, the present invention features a method of treating deep vein thrombosis in a subject that has been previously administered an anticoagulant, comprising administering to the subject an effective amount of a composition described herein (e.g., a composition comprising Compound 1 or a pharmaceutically acceptable salt thereof). In some embodiments, the anticoagulant was administered parenterally for 5-10 days.

In one aspect, the present invention features a method of treating a pulmonary embolism in a subject that has been previously administered an anticoagulant, comprising administering to the subject an effective amount of a composition described herein (e.g., a composition comprising Compound 1 or a pharmaceutically acceptable salt thereof). In some embodiments, the anticoagulant was administered parenterally for 5-10 days.

In one aspect, the present invention is directed to a method of treating a subject that has had an ischemic event (e.g., transient ischemia), comprising: a composition described herein (e.g., a composition comprising Compound 1 or a pharmaceutically acceptable salt thereof) to the subject. In some embodiments, the compound is administered to the subject within 24 hours or less, e.g., 12, 10, 9, 8, 7, 6 hours or less, after the onset of the ischemic event in the subject.

In one aspect, the present invention is directed to a method of treating a subject that has had an ischemic event (e.g., transient ischemia), comprising: administering a composition described herein (e.g., a composition comprising Compound 1 or a pharmaceutically acceptable salt thereof) to the subject. In some embodiments, the composition is administered to the subject within more than 2 hours to 12 hours, e.g., more than 2 hours to 10 hours or less, more than 2 hours to 8 hours or less, after the onset of the ischemic event in the subject.

In one aspect, the present invention is directed to a method of treating hypertension, e.g., arterial hypertension, in a subject, comprising administering to the subject an effective amount of a composition described herein (e.g., a composition comprising Compound 1 or a pharmaceutically acceptable salt thereof). In some embodiments, the hypertension, e.g., arterial hypertension, results in atherosclerosis. In some embodiments, the hypertension is pulmonary arterial hypertension.

In one aspect, the present invention is directed to a method of reducing the risk of hypertension, e.g., arterial hypertension, in a subject, comprising administering to the subject an effective amount of a composition described herein (e.g., a composition comprising Compound 1 or a pharmaceutically acceptable salt thereof). In some embodiments, the hypertension, e.g., arterial hypertension, results in atherosclerosis. In some embodiments, the hypertension is pulmonary arterial hypertension.

In one aspect, the present invention is directed to a method of prophylaxis of hypertension, e.g., arterial hypertension, in a subject, comprising administering to the subject an effective amount of a composition described herein (e.g., a composition comprising Compound 1 or a pharmaceutically acceptable salt thereof). In some embodiments, the hypertension, e.g., arterial hypertension, results in atherosclerosis. In some embodiments, the hypertension is pulmonary arterial hypertension.

In one aspect, the present invention is directed to a method of reducing inflammation in a subject, comprising administering to the subject an effective amount of a composition described herein (e.g., a composition comprising Compound 1 or a pharmaceutically acceptable salt thereof). In some embodiments, the inflammation is vascular inflammation. In some embodiments, the vascular inflammation is accompanied by atherosclerosis. In some embodiments, the vascular inflammation is accompanied by a thromboembolic disease in the subject. In some embodiments, the vascular inflammation is angiotensin II-induced vascular inflammation.

In one aspect, the present invention is directed to a method of preventing vascular leukocyte infiltration in a subject, comprising administering to the subject an effective amount of a composition described herein (e.g., a composition comprising Compound 1 or a pharmaceutically acceptable salt thereof).

In one aspect, the present invention is directed to a method of preventing angiotensin II-induced endothelial dysfunction in a subject, comprising administering to the subject an effective amount of a composition described herein (e.g., a composition comprising Compound 1 or a pharmaceutically acceptable salt thereof).

In one aspect, the present invention is directed to a method of preventing thrombin propagation in a subject, comprising administering to the subject an effective amount of a composition described herein (e.g., a composition comprising Compound 1 or a pharmaceutically acceptable salt thereof). In some embodiments, the thrombin propagation occurs on platelets.

In one aspect, the present invention is directed to a method of treating hypertension-associated renal dysfunction in a subject, comprising administering to the subject an effective amount of a composition described herein (e.g., a composition comprising Compound 1 or a pharmaceutically acceptable salt thereof).

In one aspect, the present invention is directed to a method of prophylaxis of hypertension-associated renal dysfunction in a subject, comprising administering to the subject an effective amount of a composition described herein (e.g., a composition comprising Compound 1 or a pharmaceutically acceptable salt thereof).

In one aspect, the present invention is directed to a method of reducing the risk of hypertension-associated renal dysfunction in a subject, comprising administering to the subject an effective amount of a composition described herein (e.g., a composition comprising Compound 1 or a pharmaceutically acceptable salt thereof).

In one aspect, the present invention is directed to a method of treating kidney fibrosis in a subject, comprising administering to the subject an effective amount of a composition described herein (e.g., a composition comprising Compound 1 or a pharmaceutically acceptable salt thereof).

In one aspect, the present invention is directed to a method of prophylaxis of kidney fibrosis in a subject, comprising administering to the subject an effective amount of a composition described herein (e.g., a composition comprising Compound 1 or a pharmaceutically acceptable salt thereof).

In one aspect, the present invention is directed to a method of reducing the risk of kidney fibrosis in a subject, comprising administering to the subject an effective amount of a composition described herein (e.g., a composition comprising Compound 1 or a pharmaceutically acceptable salt thereof).

In one aspect, the present invention is directed to a method of treating kidney injury in a subject, comprising administering to the subject an effective amount of a composition described herein (e.g., a composition comprising Compound 1 or a pharmaceutically acceptable salt thereof).

In one aspect, the present invention is directed to a method of prophylaxis of kidney injury in a subject, comprising administering to the subject an effective amount of a composition described herein (e.g., a composition comprising Compound 1 or a pharmaceutically acceptable salt thereof).

In one aspect, the present invention is directed to a method of reducing the risk of kidney injury in a subject, comprising administering to the subject an effective amount of a composition described herein (e.g., a composition comprising Compound 1 or a pharmaceutically acceptable salt thereof).

In one aspect, the present invention is directed to a method of inhibiting Factor XIa in a subject, comprising administering to the subject that has suffered ischemia an effective amount of a composition described herein (e.g., a composition comprising Compound 1 or a pharmaceutically acceptable salt thereof). In some embodiments, the ischemia is coronary ischemia.

In some embodiments, the subject is a mammal (e.g., a human).

In some embodiments, the subject is undergoing surgery (e.g., knee replacement surgery or hip replacement surgery). In some embodiments, the ischemia is coronary ischemia. In some embodiments, the subject is a subject with non-valvular atrial fibrillation. In some embodiments, the subject has one or more of the following risk factors for stroke: a prior stroke (e.g., ischemic, unknown, hemorrhagic), transient ischemic attack, or non-CNS systemic embolism. In some embodiments, the subject has one or more of the following risk factors for stroke: 75 years or older of age, hypertension, heart failure or left ventricular ejection fraction (e.g., less than or equal to 35%), or diabetes mellitus.

In some embodiments, the composition is administered by oral or parenteral (e.g., intravenous) administration. In some embodiments, the composition is administered by oral administration. In some embodiments, the composition is administered by parenteral (e.g., intravenous) administration. In some embodiments, the composition is administered by subcutaneous administration.

In some embodiments, the composition is administered prior to an ischemic event (e.g., to a subject is at risk of an ischemic event).

In some embodiments, the composition is administered after an ischemic event (e.g., a transient ischemic event). In some embodiments, the composition is administered about 1, 2, 3, 4, 5, 6, 7, 8, 9, 10, 11, 12, 13, or 14 days or more after an ischemic event (e.g., a transient ischemic event). In some embodiments, the composition is administered about 1, 2, 3, 4, 5, 6, 7, or 8 weeks or more after an ischemic event (e.g., a transient ischemic event).

In some embodiments, the composition is administered in combination with an additional therapeutic agent. In some embodiments, the additional therapeutic agent is administered after administration of the composition. In some embodiments, the additional therapeutic agent is administered orally. In some embodiments, the additional therapeutic agent is administered at least 1, 2, 3, 4, 5, 6, 7, 8, 10, 12, 14, 16, 18, 20, or 24 hours or more after administration of the composition. In some embodiments, the additional therapeutic agent is administered at least 1, 2, 3, 4, 5, 6, 7, 14, 21, or 28 days or more after administration of the composition. In some embodiments, the additional therapeutic agent is administered about 1 day, about 2 days, about 3 days, about 4 days, about 5 days, about 6 days, about 7 days or more after administration of the composition.

In some embodiments, the additional therapeutic agent is administered chronically (e.g., for about 1 day, about 2 days, about 3 days, about 4 days, about 5 days, about 6 days, about 7 days, about 8 days, about 9 days, about 10 days, about 11 days, about 12 days, about 13 days, or about 14 days or more) after administration of the composition.

In some embodiments, the additional therapeutic agent treats a side effect (e.g., active pathological bleeding or severe hypersensitivity reactions (e.g., anaphylactic reactions), spinal and or epidural hematoma, gastrointestinal disorder (e.g., abdominal pain upper, dyspepsia, toothache), general disorders and administration site conditions (e.g., fatigue), infections and infestations (e.g., sinusitis, urinary tract infection), musculoskeletal and connective tissues disorders (e.g., back pain, osteoarthritis), respiratory, thoracic and mediastinal disorders (e.g., oropharyngeal pain), injury, poisoning, and procedural complications (e.g., wound secretion), musculoskeletal and connective tissues disorders (e.g., pain in extremity, muscle spasm), nervous system disorders (e.g., syncope), skin and subcutaneous tissue disorders (e.g., pruritus, blister), blood and lymphatic system disorders (e.g., agranulocytosis), gastrointestinal disorders (e.g., retroperitoneal hemorrhage), hepatobiliary disorders (e.g., jaundice, cholestasis, cytolytic hepatitis), immune system disorders (e.g., hypersensitivity, anaphylactic reaction, anaphylactic shock, angioedema), nervous system disorders (e.g., cerebral hemorrhage, subdural hematoma, epidural hematoma, hemiparesis), skin and subcutaneous tissue disorders (e.g., Stevens-Johnson syndrome).

In some embodiments, the additional therapeutic agent is a NSAID (e.g., aspirin or naproxen), platelet aggregation inhibitor (e.g., clopidogrel), or anticoagulant (e.g., warfarin or enoxaparin).

In some embodiments, the additional therapeutic agent results in an additive therapeutic effect. In some embodiments, the additional therapeutic agent results in a synergistic therapeutic effect.

In another aspect, the present invention features a method of modulating (e.g., inhibiting) Factor XIa in a patient. The method comprises the step of administering an effective amount of a composition described herein (e.g., a composition comprising Compound 1 or a pharmaceutically acceptable salt thereof) to a patient in need thereof, thereby modulating (e.g., inhibiting) Factor XIa.

In another aspect, the present invention features a method of treating a subject in need thereof for a thromboembolic disorder. The method comprises administering to the subject an effective amount of a composition described herein (e.g., a composition comprising Compound 1 or a pharmaceutically acceptable salt thereof). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another aspect, the present invention features a method of prophylaxis of a thromboembolic disorder in a subject. The method comprises administering to the subject an effective amount of a composition described herein (e.g., a composition comprising Compound 1 or a pharmaceutically acceptable salt thereof). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another aspect, the present invention features a method of reducing the risk of a thromboembolic disorder in a subject. The method comprises administering to the subject an effective amount of a composition described herein (e.g., a composition comprising Compound 1 or a pharmaceutically acceptable salt thereof). The thromboembolic disorder can be arterial cardiovascular thromboembolic disorders, arterial thrombosis, venous cardiovascular thromboembolic disorders, and thromboembolic disorders in the chambers of the heart; including unstable angina, an acute coronary syndrome, first myocardial infarction, recurrent myocardial infarction, ischemia (e.g., coronary ischemia, ischemic sudden death, or transient ischemic attack), stroke (e.g., large vessel acute ischemic stroke), atherosclerosis, peripheral occlusive arterial disease, venous thromboembolism, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In one aspect, the present invention is directed to a method of treating end-stage renal disease in a subject, comprising administering to the subject an effective amount of a composition described herein (e.g., a composition comprising Compound 1 or a pharmaceutically acceptable salt thereof).

In one aspect, the present invention is directed to a method of prophylaxis of end-stage renal disease in a subject, comprising administering to the subject an effective amount of a composition described herein (e.g., a composition comprising Compound 1 or a pharmaceutically acceptable salt thereof).

In one aspect, the present invention is directed to a method of reducing the risk of end-stage renal disease in a subject, comprising administering to the subject an effective amount of a composition described herein (e.g., a composition comprising Compound 1 or a pharmaceutically acceptable salt thereof).

In another aspect, the present invention features a method of treating a thromboembolic disorder in a subject need thereof, the method comprising administering to the subject an effective amount of a composition described herein (e.g., a composition comprising Compound 1 or a pharmaceutically acceptable salt thereof), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of reducing the risk of a thromboembolic disorder in a subject need thereof, the method comprising administering to the subject an effective amount of a composition described herein (e.g., a composition comprising Compound 1 or a pharmaceutically acceptable salt thereof), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of prophylaxis of a thromboembolic disorder in a subject need thereof, the method comprising administering to the subject an effective amount of a composition described herein (e.g., a composition comprising Compound 1 or a pharmaceutically acceptable salt thereof), wherein the subject is exposed to an artificial surface. In some embodiments, the artificial surface contacts the subject's blood. In some embodiments, the artificial surface is an extracorporeal surface. In some embodiments, the artificial surface is that of an implantable device, e.g., a mechanical valve. In some embodiments, the artificial surface is that of a dialysis catheter. In some embodiments, the artificial surface is that of a cardiopulmonary bypass circuit. In some embodiments, the artificial surface is that of an artificial heart valve. In some embodiments, the artificial surface is that of a ventricular assist device. In some embodiments, the artificial surface is that of a small caliber graft. In some embodiments, the artificial surface is that of a central venous catheter. In some embodiments, the artificial surface is that of an extracorporeal membrane oxygenation (ECMO) apparatus. In some embodiments, the artificial surface causes or is associated with the thromboembolic disorder. In some embodiments, the thromboembolic disorder is a venous thromboembolism. In some embodiments, the thromboembolic disorder is deep vein thrombosis. In some embodiments, the thromboembolic disorder is pulmonary embolism.

In another aspect, the present invention features a method of treating atrial fibrillation, in a subject in need thereof, the method comprising administering to the subject an effective amount of a composition described herein (e.g., a composition comprising Compound 1 or a pharmaceutically acceptable salt thereof). In some embodiments, the subject is also in need of dialysis, e.g., renal dialysis. In some embodiments, the composition described herein is administered to the subject while the subject is undergoing dialysis. In some embodiments, the composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of reducing the risk of atrial fibrillation, in a subject in need thereof, the method comprising administering to the subject an effective amount of a composition described herein (e.g., a composition comprising Compound 1 or a pharmaceutically acceptable salt thereof). In some embodiments, the subject is at a high risk of developing atrial fibrillation. In some embodiments, the subject is also in need of dialysis, e.g., renal dialysis. In some embodiments, the composition described herein is administered to the subject while the subject is undergoing dialysis. In some embodiments, the composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of prophylaxis of atrial fibrillation, in a subject in need thereof, the method comprising administering to the subject an effective amount of a composition described herein (e.g., a composition comprising Compound 1 or a pharmaceutically acceptable salt thereof). In some embodiments, the subject is at a high risk of developing atrial fibrillation. In some embodiments, the subject is also in need of dialysis, e.g., renal dialysis. In some embodiments, the composition described herein is administered to the subject while the subject is undergoing dialysis. In some embodiments, the composition is administered to the subject before or after receiving dialysis. In some embodiments, the patient has end-stage renal disease. In some embodiments, the subject is not in need of dialysis, e.g., renal dialysis. In some embodiments, the patient is at a high risk for bleeding. In some embodiments, the atrial fibrillation is associated with another thromboembolic disorder, e.g., a blood clot.

In another aspect, the present invention features a method of treating heparin-induced thrombocytopenia in a subject in need thereof, the method comprising administering to the subject an effective amount of a composition described herein (e.g., a composition comprising Compound 1 or a pharmaceutically acceptable salt thereof).

In another aspect, the present invention features a method of reducing the risk of heparin-induced thrombocytopenia in a subject in need thereof, the method comprising administering to the subject an effective amount of a composition described herein (e.g., a composition comprising Compound 1 or a pharmaceutically acceptable salt thereof).

In another aspect, the present invention features a method of prophylaxis of heparin-induced thrombocytopenia in a subject in need thereof, the method comprising administering to the subject an effective amount of a composition described herein (e.g., a composition comprising Compound 1 or a pharmaceutically acceptable salt thereof).

In another aspect, the present invention features a method of treating heparin-induced thrombocytopenia thrombosis in a subject in need thereof, the method comprising administering to the subject an effective amount of a composition described herein (e.g., a composition comprising Compound 1 or a pharmaceutically acceptable salt thereof).

In another aspect, the present invention features a method of reducing the risk of heparin-induced thrombocytopenia thrombosis in a subject in need thereof, the method comprising administering to the subject an effective amount of a composition described herein (e.g., a composition comprising Compound 1 or a pharmaceutically acceptable salt thereof).

In another aspect, the present invention features a method of prophylaxis of heparin-induced thrombocytopenia thrombosis in a subject in need thereof, the method comprising administering to the subject an effective amount of a composition described herein (e.g., a composition comprising Compound 1 or a pharmaceutically acceptable salt thereof).

In another aspect, the present invention features a method of prophylaxis of a thromboembolic disorder in a subject in need thereof, the method comprising administering to the subject an effective amount of a composition described herein (e.g., a composition comprising Compound 1 or a pharmaceutically acceptable salt thereof), wherein the subject has cancer or is being with a chemotherapeutic. In some embodiments, the subject is concurrently receiving chemotherapy. In some embodiments, the subject has elevated lactase dehydrogenase levels. In some embodiments, the thromboembolic disorder is venous thromboembolism. In some embodiments, the thromboembolic disorder is deep vein thrombosis. In some embodiments, the thromboembolic disorder is pulmonary embolism.

In another aspect, the present invention features a method of treating thrombotic microangiopathy in a subject in need thereof, the method comprising administering to the subject an effective amount of a composition described herein (e.g., a composition comprising Compound 1 or a pharmaceutically acceptable salt thereof). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of reducing the risk of thrombotic microangiopathy in a subject in need thereof, the method comprising administering to the subject an effective amount of a composition described herein (e.g., a composition comprising Compound 1 or a pharmaceutically acceptable salt thereof). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of prophylaxis of thrombotic microangiopathy in a subject in need thereof, the method comprising administering to the subject an effective amount of a composition described herein (e.g., a composition comprising Compound 1 or a pharmaceutically acceptable salt thereof). In some embodiments, the thrombotic microangiopathy is hemolytic uremic syndrome (HUS). In some embodiments, the thrombotic microangiopathy is thrombotic thrombocytopenic purpura (TTP).

In another aspect, the present invention features a method of prophylaxis of recurrent ischemia in a subject in need thereof, the method comprising administering to the subject an effective amount of a composition described herein (e.g., a composition comprising Compound 1 or a pharmaceutically acceptable salt thereof), wherein the subject has acute coronary syndrome. In some embodiments, the subject has atrial fibrillation. In some embodiments, the subject does not have atrial fibrillation. In another aspect, the present invention features a method of treating a subject identified as being at risk, e.g., high risk, for stroke (e.g., large vessel acute ischemic stroke) or thrombosis thereby reducing the likelihood of stroke (e.g., large vessel acute ischemic stroke) or thrombosis in the subject. In some embodiments, the subject is further identified as being at risk for bleeding (e.g., excessive bleeding) or sepsis. In some embodiments, the treatment is effective without bleeding liabilities. In some embodiments, the treatment is effective to maintain the patency of infusion ports and lines. In addition, the compositions described herein are useful in the treatment and prevention of other diseases in which the generation of thrombin has been implicated as playing a physiologic role. For example, thrombin has been implicated in contributing to the morbidity and mortality of chronic and degenerative diseases, such as cancer, arthritis, atherosclerosis, vascular dementia, and Alzheimer's disease, by its ability to regulate many different cell types through specific cleavage and activation of a cell surface thrombin receptor, mitogenic effects, diverse cellular functions such as cell proliferation, for example, abnormal proliferation of vascular cells resulting in restenosis or angiogenesis, release of PDGF, and DNA synthesis. Inhibition of Factor XIa effectively blocks thrombin generation and therefore neutralizes any physiologic effects of thrombin on various cell types. The representative indications discussed above include some, but not all, of the potential clinical situations amenable to treatment with a Factor XIa inhibitor.

In another aspect, the present invention features a method of treating a subject that has edema (e.g., angioedema, e.g., hereditary angioedema), comprising administering an effective amount of a composition described herein (e.g., a composition comprising Compound 1 or a pharmaceutically acceptable salt thereof) to the subject.

In another aspect, the present invention features a method of prophylaxis of edema (e.g., angioedema, e.g., hereditary angioedema) in a subject, comprising administering an effective amount of a composition described herein (e.g., a composition comprising Compound 1 or a pharmaceutically acceptable salt thereof) to the subject.

In another aspect, the present invention features a method of reducing the risk of edema (e.g., angioedema, e.g., hereditary angioedema) in a subject, comprising administering an effective amount of a composition described herein (e.g., a composition comprising Compound 1 or a pharmaceutically acceptable salt thereof) to the subject.

In another aspect, the present invention features a method of inhibiting kallikrein in a subject, comprising administering to the subject with edema (e.g., angioedema, e.g., hereditary angioedema), an effective amount of a composition described herein (e.g., a composition comprising Compound 1 or a pharmaceutically acceptable salt thereof) to the subject.

In another aspect, the present invention features a method of treating a thromboembolic consequence or complication in a subject, comprising administering to a subject an effective amount of a composition described herein (e.g., a composition comprising Compound 1 or a pharmaceutically acceptable salt thereof). In some embodiments, the thromboembolic consequence or complication is associated with a peripheral vascular intervention (e.g., of the limbs), hemodialysis, catheter ablation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the present invention features a method of prophylaxis of a thromboembolic consequence or complication in a subject, comprising administering to a subject an effective amount of a composition described herein (e.g., a composition comprising Compound 1 or a pharmaceutically acceptable salt thereof). In some embodiments, the thromboembolic consequence or complication is associated with a peripheral vascular intervention (e.g., of the limbs), hemodialysis, catheter ablation, e.g., catheter ablation for atrial fibrillation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the present invention features a method of reducing the risk of a thromboembolic consequence or complication in a subject, comprising administering to a subject an effective amount of a composition described herein (e.g., a composition comprising Compound 1 or a pharmaceutically acceptable salt thereof). In some embodiments, the thromboembolic consequence or complication is associated with a peripheral vascular intervention (e.g., of the limbs), hemodialysis, catheter ablation, e.g., catheter ablation for atrial fibrillation, a cerebrovascular intervention, transplantation of an organ (e.g., liver), surgery (e.g., orthopedic surgery, lung surgery, abdominal surgery, or cardiac surgery, (e.g., open-heart surgery)), a trans-catheter aortic valve implantation, a large bore intervention used to treat an aneurysm, a percutaneous coronary intervention, or hemophilia therapy. In some embodiments, the surgery is orthopedic surgery, lung surgery, abdominal surgery, or cardiac surgery. In some embodiments, the cardiac surgery is complex cardiac surgery or lower risk cardiac surgery. In some embodiments, the thromboembolic consequence or complication is associated with a percutaneous coronary intervention.

In another aspect, the invention features a method of treating restenosis following arterial injury in a subject, comprising administering to a subject an effective amount of a composition described herein (e.g., a composition comprising Compound 1 or a pharmaceutically acceptable salt thereof). In some embodiments, the arterial injury occurs after a cranial artery stenting.

In another aspect, the present invention features a method of prophylaxis of restenosis following arterial injury in a subject, comprising administering to a subject an effective amount of a composition described herein (e.g., a composition comprising Compound 1 or a pharmaceutically acceptable salt thereof). In some embodiments, the arterial injury occurs after a cranial artery stenting.

In another aspect, the present invention features a method of reducing the risk of restenosis following arterial injury in a subject, comprising administering to a subject an effective amount of a composition described herein (e.g., a composition comprising Compound 1 or a pharmaceutically acceptable salt thereof). In some embodiments, the arterial injury occurs after a cranial artery stenting.

In another aspect, the present invention features a method of treating hepatic vessel thrombosis in a subject, comprising administering to a subject an effective amount of a composition described herein (e.g., a composition comprising Compound 1 or a pharmaceutically acceptable salt thereof).

In another aspect, the present invention features a method of prophylaxis of hepatic vessel thrombosis in a subject, comprising administering to a subject an effective amount of a composition described herein (e.g., a composition comprising Compound 1 or a pharmaceutically acceptable salt thereof).

In another aspect, the present invention features a method of reducing the risk of hepatic vessel thrombosis in a subject, comprising administering to a subject an effective amount of a composition described herein (e.g., a composition comprising Compound 1 or a pharmaceutically acceptable salt thereof).

In another aspect, the present invention features a method of treating a non-ST-elevation myocardial infarction or ST-elevation myocardial infarction), comprising administering to a subject an effective amount of a composition described herein (e.g., a composition comprising Compound 1 or a pharmaceutically acceptable salt thereof).

In another aspect, the present invention features a method of prophylaxis of a non-ST-elevation myocardial infarction or ST-elevation myocardial infarction in a subject, comprising administering to the subject an effective amount of a composition described herein (e.g., a composition comprising Compound 1 or a pharmaceutically acceptable salt thereof).

In another aspect, the present invention features a method of reducing the risk of a non-ST-elevation myocardial infarction or ST-elevation myocardial infarction in a subject, comprising administering to the subject an effective amount of a composition described herein (e.g., a composition comprising Compound 1 or a pharmaceutically acceptable salt thereof).

In another aspect, the present invention features a method of maintaining blood vessel patency, comprising administering to a subject an effective amount of a composition described herein (e.g., a composition comprising Compound 1 or a pharmaceutically acceptable salt thereof). In some embodiments, the subject has acute kidney injury. In some embodiments, the subject additionally undergoes continuous renal replacement therapy.

In some embodiments of any of the foregoing, the composition described herein is administered orally or parenterally. In certain embodiments, the composition described herein is administered parenterally. In certain embodiments, the composition described herein is administered after the subject has discontinued use of a direct oral anticoagulant. In certain embodiments, the subject used the direct oral anticoagulant for up to about 2.5 years. In certain embodiments, the subject is a mammal, e.g., a human.

In some embodiments of the methods described herein, the pharmaceutically acceptable salt of Compound 1 is a hydrochloride salt. In some embodiments, the composition is administered to the subject intravenously. In some embodiments, the composition is administered to the subject subcutaneously. In some embodiments, the composition is administered to the subject as a continuous intravenous infusion. In some embodiments, the composition is administered to the subject as a bolus. In some embodiments, the subject is a human. In some embodiments, the subject has an elevated risk of a thromboembolic disorder. In some embodiments, the thromboembolic disorder is a result of a complication in surgery.

In some embodiments, the subject is sensitive to or has developed sensitivity to heparin. In some embodiments, the subject is resistant to or has developed resistance to heparin. In some embodiments, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

### BRIEF DESCRIPTION OF THE DRAWINGS

FIG. 1 depicts an exemplary HPLC chromatogram of Compound 1 including baseline detail.

FIG. 2A depicts exemplary pH-development data of Compound 1 over the 10-day stability experiment at 4° C.

FIG. 2B depicts exemplary pH-development data of Compound 1 over the 10-day stability experiment at 40° C.

FIG. 3A depicts exemplary recovery data of Compound 1 over a 10-day stability assessment at 4° C.

FIG. 3B depicts exemplary recovery data of Compound 1 over a 10-day stability assessment 40° C.

FIG. 4A depicts an exemplary powder X-Ray diffractogram of Compound 1.HCl on scale.

FIG. 4B depicts an exemplary powder X-Ray diffractogram of Compound 1.HCl on d-scale.

FIG. 5 depicts the lyophilization cycle parameters developed for Compound 1.

FIG. 6 depicts an exemplary monitoring of product temperature and product drying.

FIG. 7 depicts exemplary long-term stability study of the Compound 1 lyophilized drug product at T=−80° C.

FIG. 8 depicts exemplary long-term stability study of the Compound 1 lyophilized drug product at T=−20° C.

FIG. 9 depicts exemplary long-term stability study of the Compound 1 lyophilized drug product at T=2-8° C.

FIG. 10 depicts an exemplary chromatograph of 48-hour stability sample of Compound 1 formulation diluted into normal saline.

FIG. 11 depicts the pressure gradient across membrane oxygenator for cardiopulmonary bypass experiment conducted in the hound model.

FIG. 12 depicts a comparison of plasma concentrations and activated partial thromboplastin time (aPTT) ratio measured in the hound model.

FIG. 13 depicts the activated partial thromboplastin time (aPTT) measured in the hound model following Compound 1 administration.

### DETAILED DESCRIPTION

Described herein are pharmaceutical compositions comprising Compound 1 or a pharmaceutically acceptable salt thereof, a cyclodextrin, and an excipient, methods of their use and administration, methods for their preparation, and containers comprising the solutions or mixtures.

### Definitions

As used herein, the terms “stabilized” and “stable” solutions described herein (e.g., an aqueous solution comprising Compound 1) refer to solutions that are “chemically stable” and “physically stable.” For example, a solution comprising Compound 1 is chemically stable if Compound 1 does not undergo chemical transformation (e.g., hydrolysis) or degradation (e.g., racemization, epimerization, oxidation).

“Assay”, as used herein, refers to a specific, stability-indicating procedure that determines the content of the drug substance. For example, an assay can be a chromatographic method (e.g., HPLC) involving use of a reference standard.

“Purity”, as used herein, refers to the absence of impurities, for example in a solution or composition, relative to its parent (e.g., at time=0).

“Sterilization”, as used herein, refers to aseptic fill (e.g., aseptic sterilization) or terminal sterilization.

A “reconstituted solution,” “reconstituted formulation,” or “reconstituted drug product” as used herein, refers to a solution which has been prepared by dissolving a lyophilized drug product in a diluent, such that the drug product is dissolved in an aqueous solution suitable for administration (e.g., parenteral administration).

The term “diluent” as used herein, refers to a pharmaceutically acceptable (e.g., safe and non-toxic for administration to a human) diluting substance useful for the preparation of a reconstituted solution. Exemplary diluents include sterile water for injection (WFI), a pH buffered solution (e.g., phosphate-buffered saline), sterile saline solution, or dextrose solution (e.g., 5% dextrose).

The term “osmolarity,” as used herein, refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na+ and Cl− in solution and thus provides 2 moles of dissolved components per 1 mole of dissolved NaCl in solution. Physiological osmolarity is typically in the range of about 280 mOsm/L to about 310 mOsm/L.

As used herein, “slurrying” refers to a method wherein a compound as described herein is suspended in a solvent (e.g., polar aprotic solvent or nonpolar solvent) and is collected again (e.g., by filtration) after agitating the suspension.

As used herein, “crystalline” refers to a solid having a highly regular chemical structure. The molecules are arranged in a regular, periodic manner in the 3-dimensional space of the lattice.

The term “substantially crystalline” refers to forms that may be at least a particular weight percent crystalline. Particular weight percentages are 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, Compound 1 can be a substantially crystalline sample of any of the crystalline solid forms described herein.

The term “substantially pure” relates to the composition of a specific crystalline solid form of Compound 1 that may be at least a particular weight percent free of impurities and/or other solid forms of Compound 1 or a pharmaceutically acceptable salt thereof. Particular weight percentages are 70%, 75%, 80%, 85%, 90%, 95%, 99%, or any percentage between 70% and 100%. In some embodiments, a crystalline solid form of Compound 1 or a pharmaceutically acceptable salt thereof as described herein is substantially pure at a weight percent between 95% and 100%, e.g., about 95%, about 96%, about 97%, about 98%, about 99%, or about 99.9%.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also, “therapeutic treatment”).

As used herein, and unless otherwise specified, a “therapeutically effective amount” of a composition is an amount sufficient to provide a therapeutic benefit in the treatment of a disease, disorder or condition, or to delay or minimize one or more symptoms associated with the disease, disorder or condition. A therapeutically effective amount of a composition means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a composition is an amount sufficient to prevent a disease, disorder or condition, or one or more symptoms associated with the disease, disorder or condition, or prevent its recurrence. A prophylactically effective amount of a composition means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

Disease, disorder, and condition are used interchangeably herein.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. In some embodiments, the pediatric subject is between the age of 0 and 18 years old. In some embodiments, the adult subject is beyond 18 years old.

As used herein, the term “artificial surface” refers to any non-human or non-animal surface that comes into contact with blood of the subject, for example, during a medical procedure. It can be a vessel for collecting or circulating blood of a subject outside the subject's body. It can also be a stent, valve, intraluminal catheter or a system for pumping blood. By way of non-limiting example such artificial surfaces can be steel, any type of plastic, glass, silicone, rubber, etc. In some embodiments, the artificial surface is exposed to at least 50%. 60%, 70% 80%, 90% or 100% of the blood of subject.

As used herein, the term “conditioning” or “conditioned” with respect to an artificial surface refers to priming or flushing the artificial surface (e.g., extracorporeal surface) with a composition described herein, already in a priming or flushing solution (e.g., blood, a saline solution, Ringer's solution) or as a separate administration to the artificial surface prior to, during, or after a medical procedure.

### Bulking Agent

The term “bulking agent” as used herein, includes agents that provide the structure of the composition (e.g., in lyophilized product) without interacting directly (e.g, chemically) with the pharmaceutical product (e.g, drug product). In addition to providing a pharmaceutically elegant cake, bulking agents may also impart useful qualities in regard to modifying the collapse temperature, providing freeze-thaw protection, and enhancing the active pharmaceutical ingredient (API) stability over long-term storage. Non-limiting examples of bulking agents include a sugar (e.g., a saccharide (e.g., monosaccharide, disaccharide, or polysaccharide)) or a sugar alcohol (e.g., sucrose, lactose, trehalose, dextran, erythritol, arabitol, xylitol, sorbitol, or mannitol, or a combination thereof). Bulking agents may be crystalline (e.g., mannitol, glycine, or sodium chloride) or amorphous (e.g., dextran, hydroxyethyl starch).

Preferably, the bulking agent applied in pharmaceutical formulation promotes the formation of a cake that is aesthetically acceptable, uniform, or mechanically strong. Bulking agents may also preferably promote ease and speed of reconstitution. Bulking agents may also preferably reduce or prevent cake collapse, eutectic melting, or retention of residual moisture. In some embodiments, the bulking agent is a lyoprotectant.

### Buffers

In some embodiments, the aqueous pharmaceutical compositions described herein further comprise a buffer (e.g., a buffer at a pH of between about 6 and about 8 (e.g., between about 6.5 and about 7.0, or about 6.8).

As used herein, the terms “buffer,” “buffer system,” or “buffering component” refers to a compound that, usually in combination with at least one other compound, provides a chemical system in solution that exhibits buffering capacity, that is, the capacity to neutralize, within limits, the pH lowering or raising effects of either strong acids or bases (alkali), respectively, with relatively little or no change in the original pH (e.g., the pH before being affected by, e.g., strong acid or base). For example, a buffer described herein maintains or controls the pH of a solution to a certain pH range. For example, “buffering capacity” can refer to the millimoles (mM) of strong acid or base (or respectively, hydrogen or hydroxide ions) required to change the pH by one unit when added to one liter (a standard unit) of the buffer solution. From this definition, it is apparent that the smaller the pH change in a solution caused by the addition of a specified quantity of acid or alkali, the greater the buffer capacity of the solution. See, for example, Remington: The Science and Practice of Pharmacy, Mack Publishing Co., Easton, Pa. (19^{th} Edition, 1995), Chapter 17, pages 225-227. The buffer capacity will depend on the kind and concentration of the buffer components.

In some embodiments, the buffer comprises a monoprotic acid. In some embodiments, the buffer comprises a polyprotic acid (e.g., maleate, citrate, or phosphate). In some embodiments, the buffer is a solution of one or more substances (e.g., a salt of a weak acid and a weak base; a mixture of a weak acid and a salt of the weak acid with a strong base).

In some embodiments, the buffer is maleate buffer. In some embodiments, the buffer is citrate buffer. In some embodiments, the buffer is phosphate buffer.

### Lyoprotectant

The term “lyoprotectant” as used herein, refers to a substance, when combined with the drug product, reduces the chemical and/or physical instability of the drug product upon lyophilization and/or subsequent storage. Exemplary lyoprotectants include sugars and their corresponding sugar alcohols, such as sucrose, lactose, trehalose, dextran, erythritol, arabitol, xylitol, sorbitol, and mannitol; amino acids, such as arginine or histidine; lyotropic salts, such as magnesium sulfate; polyols, such as propylene glycol, glycerol, poly(ethylene glycol), or polypropylene glycol); and combinations thereof. Additional exemplary lyoprotectants include gelatin, dextrins, modified starch, and carboxymethyl cellulose. Sugar alcohols are those compounds obtained by reduction of mono- and di-saccharides, such as lactose, trehalose, maltose, lactulose, and maltulose.

### Cyclodextrins

Cyclodextrins are cyclic oligosaccharides containing or comprising six (α-cyclodextrin), seven (β-cyclodextrin), eight (γ-cyclodextrin), or more α-(1,4)-linked glucose residues. The hydroxyl groups of the cyclodextrins are oriented to the outside of the ring while the glucosidic oxygen and two rings of the non-exchangeable hydrogen atoms are directed towards the interior of the cavity.

The cyclodextrin may be chemically modified such that some or all of the primary or secondary hydroxyl groups of the macrocycle, or both, are functionalized with a pendant group. Suitable pendant groups include, but are not limited to, sulfinyl, sulfonyl, phosphate, acyl, and C1-C12 alkyl groups optionally substituted with one or more (e.g., 1, 2, 3, or 4) hydroxy, carboxy, carbonyl, acyl, oxy, oxo; or a combination thereof. Methods of modifying these alcohol residues are known in the art, and many cyclodextrin derivatives are commercially available, including sulfo butyl ether β-cyclodextrins available under the trade name CAPTISOL® from Ligand Pharmaceuticals (La Jolla, Calif.).

Cyclodextrins include, but are not limited to, alkyl cyclodextrins, hydroxy alkyl cyclodextrins, such as hydroxy propyl β-cyclodextrin, carboxy alkyl cyclodextrins and sulfoalkyl ether cyclodextrins, such as sulfo butyl ether β-cyclodextrin.

In particular embodiments, the cyclodextrin is beta cyclodextrin having a plurality of charges (e.g., negative or positive) on the surface. In more particular embodiments, the cyclodextrin is a β-cyclodextrin containing or comprising a plurality of functional groups that are negatively charged at physiological pH. Examples of such functional groups include, but are not limited to, carboxylic acid (carboxylate) groups, sulfonate (RSO3-), phosphonate groups, phosphinate groups, and amino acids that are negatively charged at physiological pH. The charged functional groups can be bound directly to the cyclodextrins or can be linked by a spacer, such as an alkylene chain. The number of carbon atoms in the alkylene chain can be varied, but is generally between about 1 and 10 carbons, preferably 1-6 carbons, more preferably 1-4 carbons. Highly sulfated cyclodextrins are described in U.S. Pat. No. 6,316,613.

In one embodiment, the cyclodextrins is a β-cyclodextrin functionalized with a plurality of sulfobutyl ether groups. Such a cyclodextrins is sold under the trade name CAPTISOL®.

CAPTISOL® is a polyanionic beta-cyclodextrin derivative with a sodium sulfonate salt separated from the lipophilic cavity by a butyl ether spacer group, or sulfobutylether (SBE). CAPTISOL® is not a single chemical species, but comprised of a multitude of polymeric structures of varying degrees of substitution and positional/regional isomers dictated and controlled to a uniform pattern by a patented manufacturing process consistently practiced and improved to control impurities.

CAPTISOL® contains six to seven sulfobutyl ether groups per cyclodextrin molecule. Because of the very low pKa of the sulfonic acid groups, CAPTISOL® carries multiple negative charges at physiologically compatible pH values. The four-carbon butyl chain coupled with repulsion of the end group negative charges allows for an “extension” of the cyclodextrin cavity. This often results in stronger binding to drug candidates than can be achieved using other modified cyclodextrins. It also provides a potential for ionic charge interactions between the cyclodextrin and a positively charged drug molecule. In addition, these derivatives impart exceptional solubility and parenteral safety to the molecule. Relative to beta-cyclodextrin, CAPTISOL® provides higher interaction characteristics and superior water solubility in excess of 100 grams/100 ml, a 50-fold improvement.

### Solubilizing Agent

The term “solubilizing agent”, as used herein, describes a substance which is capable of facilitating the dissolution of insoluble or poorly soluble components in a solution containing same. Representative examples of solubilizing agents that are usable in the context of the present invention include, without limitation, TWEENS® and spans, e.g., TWEEN® 80 and TWEEN® 20. Other solubilizing agents that are usable in the context of embodiments of the invention include, for example, polyoxyethylene sorbitan esters, polyoxyethylene sorbitan fatty acid ester, polyoxyethylene n-alkyl ethers, polyethylene glycols (e.g, PEG200, PEG300, PEG400, PEG500, PEG600, etc), n-alkyl amine n-oxides, poloxamers, organic solvents, phospholipids and cyclodextrins.

### Containers

Also described herein are containers that include an aqueous solution or admixture described herein. Examples of containers include bags (e.g., plastic or polymer bags such as PVC), vials (e.g., a glass vial), bottles, or syringes. In an embodiment, the container is configured to deliver the solution or admixture parenterally (e.g., intramuscular, subcutaneous, or intravenous).

In some embodiments, the product intended for injection is packed in a suitably sized hermetically sealed glass container. In some embodiments the product is intended to be diluted prior to infusion, and is packaged in a pharmaceutical vial or bottle (e.g. suitably sized, suitable glass or plastic vial or bottle). In some embodiments the product may prepared to be ready for injection and may be packaged in a prefilled syringe or other syringe device (e.g. suitably sized, suitable glass or plastic package) or large volume container (e.g. suitably sized, suitable glass or plastic container) intended to be used for infusion. In some embodiments, the product is provided in a container that does not leach (e.g., does not introduce (or allow growth of) contamination or impurities in the solution.

### Lyophilization

The term “lyophilization” refers to a freeze-drying process in which water is removed from a product by freezing the product and placing it under a vacuum, which allows the ice to change directly from the solid phase to the vapor phase without passing through the liquid phase. The process consists of three separate, unique, and interdependent processes: freezing, primary drying (sublimation), and secondary drying (desorption). There are several advantages associated with lyophilization, such as: (i) ease of processing a liquid, which simplifies aseptic handling; (ii) enhanced stability of a dry powder; (iii) removal of water without excessive heating of the product; (iv) enhanced product stability in a dry state; and (v) rapid and easy dissolution of the reconstituted product.

The lyophilization process generally includes the following steps:
Dissolving the drug and excipients in a suitable solvent, generally water for injection (WFI).
Sterilizing the bulk solution by passing it through a 0.22 micron bacteria-retentive filter.
Filling into individual sterile containers and partially stoppering the containers under aseptic conditions.
Transporting the partially stoppered containers to the lyophilizer and loading into the chamber under aseptic conditions.
Freezing the solution by placing the partially stoppered containers on cooled shelves in a freeze-drying chamber or pre-freezing in another chamber.
Applying a vacuum to the chamber and heating the shelves in order to evaporate the water from the frozen state.
Complete stoppering of the vials usually by hydraulic or screw rod stoppering mechanisms installed in the lyophilizers.

### Compounds

The present invention relates, in part, to pharmaceutical compositions comprising a compound of Formula (I-A):

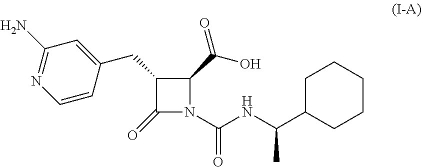 [SMILES: C[C@@H](NC(=O)N1C(=O)[C@H](Cc2ccnc(N)c2)[C@H]1C(=O)O)C1CCCCC1]

also referred to herein as “Compound 1,” or a pharmaceutically acceptable salt thereof. In some embodiments, the pharmaceutically acceptable salt of Compound 1 is a hydrochloride salt.

In some embodiments, a compound described herein is formed into a salt. A compound described herein can be administered as a free acid, a zwitterion or as a salt. A salt can also be formed between a cation and a negatively charged substituent on a compound described herein, the deprotonated carboxylic acid moiety of Compound 1 for example. Suitable cationic counterions include sodium ions, potassium ions, magnesium ions, calcium ion, and ammonium ions (e.g., a tetraalkyl ammonium cation such as tetramethylammonium ion). In acid addition salts, a salt can be formed between an anion and a positively charged substituent (e.g., amino group) or basic substituent (e.g., pyridyl) on a compound described herein. Suitable anions include chloride, bromide, iodide, sulfate, nitrate, phosphate, citrate, methanesulfonate, trifluoroacetate, and acetate.

Pharmaceutically acceptable salts of the compounds described herein (e.g., a pharmaceutically acceptable salt of Compound 1) also include those derived from pharmaceutically acceptable inorganic and organic acids and bases. Examples of suitable acid salts include acetate, 4-acetamidobenzoate, adipate, alginate, 4-aminosalicylate, aspartate, ascorbate, benzoate, benzenesulfonate, bisulfate, butyrate, citrate, camphorate, camphorsulfonate, carbonate, cinnamate, cyclamate, decanoate, decanedioate, 2,2-dichloroacetate, digluconate, dodecylsulfate, ethanesulfonate, ethane-1,2-disulfonate, formate, fumarate, galactarate, glucoheptanoate, gluconate, glucoheptonate, glucoronate, glutamate, glutarate, glycerophosphate, glycolate, hemisulfate, heptanoate, hexanoate, hippurate, hydrochloride, hydrobromide, hydroiodide, 1-hydroxy-2-naphthoate, 2-hydroxyethanesulfonate, isobutyrate, lactate, lactobionate, laurate, malate, maleate, malonate, mandelate, methanesulfonate, naphthalene-1,5-disulfonate, 2-naphthalenesulfonate, nicotinate, nitrate, octanoate, oleate, oxalate, 2-oxoglutarate, palmitate, palmoate, pectinate, 3-phenylpropionate, phosphate, phosphonate, picrate, pivalate, propionate, pyroglutamate, salicylate, sebacate, succinate, stearate, sulfate, tartrate, thiocyanate, toluenesulfonate, tosylate, and undecanoate.

Salts derived from appropriate bases include alkali metal (e.g., sodium), alkaline earth metal (e.g., magnesium), ammonium and (alkyl)₄N⁺ salts. This invention also envisions the quaternization of any basic nitrogen-containing groups of the compounds disclosed herein. Water or oil-soluble or dispersible products may be obtained by such quaternization.

As used herein, the compounds of this invention, including the Compound 1, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood), or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein.

Any formula or a compound described herein is also intended to represent unlabeled forms as well as isotopically labeled forms of the compounds, isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ⁵¹P, ³²P, ³⁵S, ³⁶Cl, ¹²⁵I respectively. The invention includes various isotopically labeled compounds as defined herein, for example, those into which radioactive isotopes, such as ³H, ¹³C, and ¹⁴C are present. Such isotopically labelled compounds are useful in metabolic studies (with ¹⁴C), reaction kinetic studies (with, for example ′H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies, isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of a formula described herein. The concentration of such a heavier isotope, specifically deuterium, may be defined by the isotopic enrichment factor. The term “isotopic enrichment factor” as used herein means the ratio between the isotopic abundance and the natural abundance of a specified isotope If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 8633.3 (99.5% deuterium incorporation).

Isotopically-labelled compounds described herein can generally be prepared by conventional techniques known to those skilled in the art or by processes analogous to those described in the accompanying Examples and Preparations using an appropriate isotopically-labeled reagents in place of the non-labeled reagent previously employed. Pharmaceutically acceptable solvates in accordance with the invention include those wherein the solvent of crystallization may be isotopically substituted, e.g, D₂O, D₆-acetone, D₆-DMSO.

Any asymmetric atom (e.g., carbon or the like) of the compound(s) of the present invention can be present in racemic or enantiomerically enriched, for example the (R)- (S)- or (RS)-configuration, in certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated bonds may, if possible, be present in cis-(Z)- or trans-(E)-form Accordingly, as used herein a compound of the present invention can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof. Any resulting mixtures of isomers can be separated on the basis of the physicochemical differences of the constituents, into the pure or substantially pure geometric or optical isomers, diastereomers, racemates, for example, by chromatography or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. An acidic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, (+)-O,O′-Di-p-toluoyl-D-tartaric acid, mandelic acid, malic acid or camphor-10-sulfonic acid. Racemic products can also be resolved by chiral chromatography, e.g., high pressure liquid chromatography (HPLC) using a chiral adsorbent.

The compounds described herein (e.g., Compound 1) may also be represented in multiple tautomeric forms. In such instances, the invention expressly includes all tautomeric forms of the compounds described herein. All crystal forms of the compounds described herein are expressly included in this invention.

### Methods of Synthesizing Compounds

The compounds described herein can be synthesized by conventional methods using commercially available starting materials and reagents. For example, compounds can be synthesized utilizing the methods set forth in U.S. Pat. No. 7,501,404, or as described in the methods described herein.

Compounds described herein can be purified using various techniques in the art of synthetic organic chemistry. A compound described herein can be purified using one or more chromatographic methods, e.g., column chromatography or HPLC. A compound described herein can be purified by a purification method that is not chromatography, e.g., recrystallization or slurrying. In one embodiment, a compound described herein can be purified using recrystallization. In another embodiment, a compound described herein can also be purified by slurrying.

In some embodiments, a compound described herein that has been purified by chromatography can also be purified by a recrystallization. A compound described herein can also be purified by slurrying (or re-slurrying) the compound with one or more solvents, e.g., a slurry described herein. A compound described herein can also be purified by trituration with one or more solvents, e.g., a trituration described herein. For example, a compound described herein that has been purified by chromatography can also be purified by trituration. In a chemical reactor, the trituration process may be affected by suspension or resuspension of a solid product in a solvent or mixture of solvents with mechanical stirring. In an embodiment, a compound described herein can also be purified by precipitation from a solution using one or more anti-solvents. For example, a compound described herein that has been purified by chromatography can also be purified by precipitation. In one embodiment, a compound described herein is purified by simulated moving bed (SMB) chromatography. In one embodiment, a compound described herein is purified by supercritical fluid chromatography, e.g., supercritical fluid chromatography with liquid carbon dioxide. In one embodiment, a compound described herein is purified by chiral chromatography e.g., high pressure liquid chromatography (HPLC) using a chiral adsorbent.

### Methods of Treatment, Prophylaxis, or Reduction of Risk

The compounds described herein (e.g., Compound 1 or a pharmaceutically acceptable salt thereof) can inhibit Factor XIa or kallikrein. In some embodiments, the compounds described herein (e.g., Compound 1 or a pharmaceutically acceptable salt thereof) can inhibit both Factor XIa and kallikrein. As a result, these compounds can be useful in the treatment, prophylaxis, or reduction in the risk of a disorder described herein.

Exemplary disorders include thrombotic events associated with coronary artery and cerebrovascular disease, venous or arterial thrombosis, coagulation syndromes, ischemia (e.g., coronary ischemia) and angina (stable and unstable), deep vein thrombosis (DVT), hepatic vein thrombosis, disseminated intravascular coagulopathy, Kasabach-Merritt syndrome, pulmonary embolism, myocardial infarction (e.g., ST-elevation myocardial infarction or non-ST-elevation myocardial infarction (e.g., non-ST-elevation myocardial infarction before catheterization), cerebral infarction, cerebral thrombosis, transient ischemic attacks, atrial fibrillation (e.g., non-valvular atrial fibrillation), cerebral embolism, thromboembolic complications of surgery (e.g., hip or knee replacement, orthopedic surgery, cardiac surgery, lung surgery, abdominal surgery, or endarterectomy) and peripheral arterial occlusion and may also be useful in treating or preventing myocardial infarction, stroke (e.g., large vessel acute ischemic stroke), angina and other consequences of atherosclerotic plaque rupture. The compounds of the invention possessing Factor XIa or kallikrein inhibition activity may also be useful in preventing thromboembolic disorders, e.g., venous thromboembolisms, in cancer patients, including those receiving chemotherapy and/or those with elevated lactase dehydrogenase (LDH) levels, and to prevent thromboembolic events at or following tissue plasminogen activator-based or mechanical restoration of blood vessel patency. The compounds of the invention possessing Factor XIa or kallikrein inhibition activity may also be useful as inhibitors of blood coagulation such as during the preparation, storage and fractionation of whole blood. Additionally, the compounds described herein may be used in acute hospital settings or periprocedurally, where a patient is at risk of a thromboembolic disorder or complication, and also in patients who are in a heightened coagulation state, e.g., cancer patients.

Factor XIa inhibition, according to the present invention, can be a more effective and safer method of inhibiting thrombosis compared to inhibiting other coagulation serine proteases such as thrombin or Factor Xa. Administration of a small molecule Factor XIa inhibitor should have the effect of inhibiting thrombin generation and clot formation with no or substantially no effect on bleeding times and little or no impairment of haemostasis. These results differ substantially from that of other “direct acting” coagulation protease inhibitors (e.g., active-site inhibitors of thrombin and Factor Xa), which demonstrate prolongation of bleeding time and less separation between antithrombotic efficacy and bleeding time prolongation. A preferred method according to the invention comprises administering to a mammal a pharmaceutical composition containing at least one compound of the invention.

The compounds described herein (e.g., Compound 1 or pharmaceutically acceptable salts thereof) can inhibit kallikrein. As a result, these compounds can be useful in the treatment, prophylaxis, or reduction in the risk of diseases involved in inflammation, such as edema (e.g., cerebral edema, macular edema, and angioedema (e.g., hereditary angioedema)). In some embodiments, the compounds of the invention can be useful in the treatment or prevention of hereditary angioedema. The compounds described herein (e.g., Compound 1) can also be useful in the treatment, prophylaxis, or reduction in the risk of, e.g., stroke, ischemia (e.g., coronary ischemia), and perioperative blood loss for example, Compound 1 or pharmaceutically acceptable salts thereof. The methods of the present invention are useful for treating or preventing those conditions which involve the action of Factor XIa or kallikrein. Accordingly, the methods of the present invention are useful in treating consequences of atherosclerotic plaque rupture including cardiovascular diseases associated with the activation of the coagulation cascade in thrombotic or thrombophilic states.

More particularly, the methods of the present invention can be used in the treatment, prophylaxis, or reduction in the risk of acute coronary syndromes such as coronary artery disease, myocardial infarction, unstable angina (including crescendo angina), ischemia (e.g., ischemia resulting from vascular occlusion), and cerebral infarction. The methods of the present invention further may be useful in the treatment, prophylaxis, or reduction in the risk of stroke (e.g., large vessel acute ischemic stroke) and related cerebral vascular diseases (including cerebrovascular accident, vascular dementia, and transient ischemic attack); venous thrombosis and thrombo-embolism, such as deep vein thrombosis (DVT) and pulmonary embolism; thrombosis associated with atrial fibrillation, ventricular enlargement, dilated cardiac myopathy, or heart failure; peripheral arterial disease and intermittent claudication; the formation of atherosclerotic plaques and transplant atherosclerosis; restenosis following arterial injury induced endogenously (by rupture of an atherosclerotic plaque), or exogenously (by invasive cardiological procedures such as vessel wall injury resulting from angioplasty or post-cranial artery stenting); disseminated intravascular coagulopathy, Kasabach-Merritt syndrome, cerebral thrombosis, and cerebral embolism.

Additionally, the methods of the present invention can be used in the treatment, prophylaxis (e.g., preventing), or reduction in the risk of thromboembolic consequences or complications associated with cancer, thrombectomy, surgery (e.g., hip replacement, orthopedic surgery), endarterectomy, introduction of artificial heart valves, peripheral vascular interventions (e.g., of the limbs), cerebrovascular interventions, large bore interventions used in the treatment of aneurysms, vascular grafts, mechanical organs, and implantation (e.g., trans-catheter aortic valve implantation) or transplantation of organs, (e.g., transplantation of the liver), tissue, or cells); percutaneous coronary interventions; catheter ablation; hemophilia therapy; hemodialysis; medications (such as tissue plasminogen activator or similar agents and surgical restoration of blood vessel patency) in patients suffering myocardial infarction, stroke (e.g., large vessel acute ischemic stroke), pulmonary embolism and like conditions; medications (such as oral contraceptives, hormone replacement, and heparin, e.g., for treating heparin-induced thrombocytopenia); sepsis (such as sepsis related to disseminated intravascular coagulation); pregnancy or childbirth; and another chronic medical condition. The methods of the present invention may be used to treat thrombosis due to confinement (e.g., immobilization, hospitalization, bed rest, or limb immobilization, e.g., with immobilizing casts, etc.). In some embodiments, the thromboembolic consequence or complication is associated with a percutaneous coronary intervention.

Additionally, the compounds described herein (e.g., Compound 1) or pharmaceutically acceptable salts thereof or compositions thereof can be useful in the treatment, prophylaxis and reduction in the risk of a thromboembolic disorder, e.g., a venous thromboembolism, deep vein thrombosis or pulmonary embolism, or associated complication in a subject, wherein the subject is exposed to an artificial surface. The artificial surface can contact the subject's blood, for example, as an extracorporeal surface or that of an implantable device. Such artificial surfaces include, but are not limited to, those of dialysis catheters, cardiopulmonary bypass circuits, artificial heart valves, e.g., mechanical heart valves (MHVs), ventricular assist devices, small caliber grafts, central venous catheters, extracorporeal membrane oxygenation (ECMO) apparatuses. Further, the thromboembolic disorder or associated complication may be caused by the artificial surface or associated with the artificial surface. For example, foreign surfaces and various components of mechanical heart valves (MHVs) are pro-thrombotic and promote thrombin generation via the intrinsic pathway of coagulation. Further, thrombin and FXa inhibitors are contraindicated with thromboembolic disorders or associated complications caused by artificial surfaces such as those MHVs, as these inhibitors are ineffective at blocking the intrinsic pathway at plasma levels that will not cause heavy bleeding. The compounds of the present invention, which can be used as, for example, Factor XIa inhibitors, are thus contemplated as alternative therapeutics for these purposes.

The compounds described herein (e.g., Compound 1) or pharmaceutically acceptable salts thereof or compositions thereof can also be useful for the treatment, prophylaxis, or reduction in the risk of atrial fibrillation in a subject in need thereof. For example, the subject can have a high risk of developing atrial fibrillation. The subject can also in need of dialysis, such as renal dialysis. The compounds described herein (e.g., Compound 1) or pharmaceutically acceptable salts thereof or compositions thereof can be administered before, during, or after dialysis. Direct oral anticoagulants (DOACs) currently available on the market, such as certain FXa or thrombin inhibitors, are contraindicated for atrial fibrillation under such a condition. The compounds of the present invention, which can be used as, for example, Factor XIa inhibitors, are thus contemplated as alternative therapeutics for these purposes. Additionally, the subject can be at a high risk of bleeding. In some embodiments, the subject can have end-stage renal disease. In other cases, the subject is not in need of dialysis, such as renal dialysis. Further, the atrial fibrillation can be associated with another thromboembolic disorder such as a blood clot.

Furthermore, the compounds described herein (e.g., Compound 1) or pharmaceutically acceptable salts thereof or compositions thereof can be used in the treatment, prophylaxis, or reduction in the risk of hypertension, e.g., arterial hypertension, in a subject. In some embodiments, the hypertension, e.g., arterial hypertension, can result in atherosclerosis. In some embodiments, the hypertension can be pulmonary arterial hypertension.

Furthermore, the compounds described herein (e.g., Compound 1) or pharmaceutically acceptable salts thereof or compositions thereof can be used in the treatment, prophylaxis, or reduction in the risk of disorders such as heparin-induced thrombocytopenia, heparin-induced thrombocytopenia thrombosis, or thrombotic microangiopathy, e.g., hemolytic uremic syndrome (HUS) or thrombotic thrombocytopenic purpura (TTP).

In some embodiments, the subject is sensitive to or has developed sensitivity to heparin. Heparin-induced thrombocytopenia (HIT) is the development of (a low platelet count), due to the administration of various forms of heparin. HIT is caused by the formation of abnormal antibodies that activate platelets. HIT can be confirmed with specific blood tests. In some embodiments, the subject is resistant to or has developed resistance to heparin. For example, activated clotting time (ACT) test can be performed on the subject to test for sensitivity or resistance towards heparin. The ACT test is a measure of the intrinsic pathway of coagulation that detects the presence of fibrin formation. A subject who is sensitive and/or resistant to standard dose of heparin typically do not reach target anticoagulation time. Common correlates of heparin resistance include, but are not limited to, previous heparin and/or nitroglycerin drips and decreased antithrombin III levels. In some embodiments, the subject has previously been administered an anticoagulant (e.g. bivalirudin/Angiomax).

The compounds described herein (e.g., Compound 1) or pharmaceutically acceptable salts thereof or compositions thereof can be used to reduce inflammation in a subject. In some embodiments, the inflammation can be vascular inflammation. In some embodiments, the vascular inflammation can be accompanied by atherosclerosis. In some embodiments, the vascular inflammation can be accompanied by a thromboembolic disease in the subject. In some embodiments, the vascular inflammation can be angiotensin II-induced vascular inflammation.

The compounds described herein (e.g., Compound 1) or pharmaceutically acceptable salts thereof or compositions thereof can be used in the treatment, prophylaxis, or reduction in the risk of renal disorders or dysfunctions, including end-stage renal disease, hypertension-associated renal dysfunction in a subject, kidney fibrosis, and kidney injury.

The methods of the present invention may also be used to maintain blood vessel patency, for example, in patients undergoing thrombectomy, transluminal coronary angioplasty, or in connection with vascular surgery such as bypass grafting, arterial reconstruction, atherectomy, vascular grafts, stent patency, and organ, tissue or cell implantation and transplantation. The inventive methods may be used to inhibit blood coagulation in connection with the preparation, storage, fractionation, or use of whole blood. For example, the inventive methods may be used in maintaining whole and fractionated blood in the fluid phase such as required for analytical and biological testing, e.g., for ex vivo platelet and other cell function studies, bioanalytical procedures, and quantitation of blood-containing components, or for maintaining extracorporeal blood circuits, as in a renal replacement solution (e.g., hemodialysis) or surgery (e.g., open-heart surgery, e.g., coronary artery bypass surgery). In some embodiments, the renal replacement solution can be used to treat patients with acute kidney injury. In some embodiments, the renal replacement solution can be continuous renal replacement therapy.

In addition, the methods of the present invention may be useful in treating and preventing the prothrombotic complications of cancer. The methods may be useful in treating tumor growth, as an adjunct to chemotherapy, for preventing angiogenesis, and for treating cancer, more particularly, cancer of the lung, prostate, colon, breast, ovaries, and bone.

### Extracorporeal Membrane Oxygenation (ECMO)

“Extracorporeal membrane oxygenation” (or “ECMO”) as used herein, refers to extracorporeal life support with a blood pump, artificial lung, and vascular access cannula, capable of providing circulatory support or generating blood flow rates adapted to support blood oxygenation, and optionally carbon dioxide removal. In venovenous ECMO, extracorporeal gas exchange is provided to blood that has been withdrawn from the venous system; the blood is then reinfused to the venous system. In venoarterial ECMO, gas exchange is provided to blood that is withdrawn from the venous system and then infused directly into the arterial system to provide partial or complete circulatory or cardiac support. Venoarterial ECMO allows for various degrees of respiratory support.

As used herein, “extracorporeal membrane oxygenation” or “ECMO” refers to extracorporeal life support that provides circulatory support or generates blood flow rates adequate to support blood oxygenation. In some embodiments, ECMO comprises removal of carbon dioxide from a subject's blood. In some embodiments, ECMO is performed using an extracorporeal apparatus selected from the group consisting of a blood pump, artificial lung, and vascular access cannula.

As used herein, “venovenous ECMO” refers to a type of ECMO in which blood is withdrawn from the venous system of a subject into an ECMO apparatus and subjected to gas exchange (including oxygenation of the blood), followed by reinfusion of the withdrawn blood into the subject's venous system. As used herein, “venoarterial ECMO” refers to a type of ECMO in which blood is withdrawn from the venous system of a subject into an ECMO apparatus and subjected to gas exchange (including oxygenation of the blood), followed by infusion of the withdrawn blood directly into the subject's arterial system. In some embodiments, venoarterial ECMO is performed to provide partial circulatory or cardiac support to a subject in need thereof. In some embodiments, venoarterial ECMO is performed to provide complete circulatory or cardiac support to a subject in need thereof.

The compounds of the present invention can be used in the treatment, prophylaxis, or reduction in the risk of a thromboembolic disorder in a subject in need thereof, wherein the subject is exposed to an artificial surface such as that of an extracorporeal membrane oxygenation (ECMO) apparatus (vide supra), which can be used as a rescue therapy in response to cardiac or pulmonary failure. The surface of an ECMO apparatus that directly contacts the subject can be a pro-thrombotic surface that can result in a thromboembolic disorder such as a venous thromboembolism, e.g., deep vein thrombosis or pulmonary embolism, leading to difficulties in treating a patient in need of ECMO. Clots in the circuit are the most common mechanical complication (19%). Major clots can cause oxygenator failure, and pulmonary or systemic emboli.

ECMO is often administered with a continuous infusion of heparin as an anticoagulant to counter clot formation. However, cannula placement can cause damage to the internal jugular vein, which causes massive internal bleeding. Bleeding occurs in 30-40% of patients receiving ECMO and can be life-threatening. This severe bleeding is due to both the necessary continuous heparin infusion and platelet dysfunction. Approximately 50% of reported deaths are due to severe bleeding complications. Aubron et al. Critical Care, 2013, 17:R73 looked at the factors associated with ECMO outcomes.

The compounds of the present invention, which can be used as, for example, Factor XIa inhibitors, are thus contemplated as an alternative replacement for heparin in ECMO therapy. The compounds of the present invention are contemplated as effective agents for blocking the intrinsic pathway at plasma levels that will afford effective anti-coagulation/anti-thrombosis without marked bleeding liabilities. In some embodiments, the subject is sensitive to or has developed sensitivity to heparin. In some embodiments, the subject is resistant to or has developed resistance to heparin.

### Ischemia

“Ischemia” or an “ischemic event” is a vascular disease generally involving vascular occlusion or a restriction in blood supply to tissues. Ischemia can cause a shortage of oxygen and glucose needed for cellular metabolism. Ischemia is generally caused by problematic blood vessels that result in damage or dysfunction of tissue. Ischemia can also refer to a local loss in blood or oxygen in a given part of the body resulting from congestion (e.g., vasoconstriction, thrombosis, or embolism). Causes include embolism, thrombosis of an atherosclerosis artery, trauma, venous problems, aneurysm, heart conditions (e.g., myocardial infarction, mitral valve disease, chronic arterial fibrillation, cardiomyopathies, and prosthesis), trauma or traumatic injury (e.g., to an extremity producing partial or total vessel occlusion), thoracic outlet syndrome, atherosclerosis, hypoglycemia, tachycardia, hypotension, outside compression of a blood vessel (e.g., by a tumor), sickle cell disease, localized extreme cold (e.g., by frostbite), tourniquet application, glutamate receptor stimulation, arteriovenous malformations, rupture of significant blood vessels supplying a tissue or organ, and anemia.

A transient ischemic event generally refers to a transient (e.g., short-lived) episode of neurologic dysfunction caused by loss of blood flow (e.g., in the focal brain, spinal cord, or retinal) without acute infarction (e.g., tissue death). In some embodiments, the transient ischemic event lasts for less than 72 hours, 48 hours, 24 hours, 12 hours, 10 hours, 8 hours, 4 hours, 2 hours, 1 hour, 45 minutes, 30 minutes, 20 minutes, 15 minutes, 10 minutes, 5 minutes, 4 minutes, 3 minutes, 2 minutes, or 1 minute.

### Angioedema

Angioedema is the rapid swelling of the dermis, subcutaneous tissue, mucosa, and submucosal tissues. Angioedema is typically classified as either hereditary or acquired.

“Acquired angioedema” can be immunologic, non-immunologic, or idiopathic; caused by e.g., allergy, as a side effect of medications, e.g., ACE inhibitor medications.

“Hereditary angioedema” or “HAE” refers to a genetic disorder that results in acute periods of edema (e.g., swelling) that may occur in nearly all parts of the body, including the face, limbs, neck, throat, larynx, extremities, gastrointestinal tract, and genitalia. Attacks of HAE can often be life-threatening, with severity depending on the area affected, e.g., abdominal attacks may result in intestinal obstruction, while swelling of the larynx and upper airway can lead to asphyxiation. Pathogenesis of hereditary angioedema may be related to unopposed activation of the contact pathway by the initial generation of kallikrein or clotting factors (e.g., Factor XII).

Signs and symptoms include swelling, e.g., of the skill of the face, mucosa of the mouth or throat, and tongue. Itchiness, pain, decreased sensation in the affected areas, urticaria (i.e., hives), or stridor of the airway may also be a sign of angioedema. However, there can be no associated itch, or urticaria, e.g., in hereditary angioedema. HAE subjects can experience abdominal pain (e.g., abdominal pain lasting one to five days, abdominal attacks increasing a subject's white blood cell count), vomiting, weakness, watery diarrhea, or rash.

Bradykinin plays an important role in angioedema, particularly hereditary angioedema. Bradykinin is released by various cell types in response to numerous different stimuli and is a pain mediator. Interfering with bradykinin production or degradation can lead to angioedema. In hereditary angioedema, continuous production of enzyme kallikrein can facilitate bradykinin formation. Inhibition of kallikrein can interfere with bradykinin production; and treat or prevent angioedema.

The methods described herein may comprise administering to a subject in need thereof an effective amount of a pharmaceutical composition described herein.

In an aspect, the methods described herein can include those in which a subject's blood is in contact with an artificial surface. For example, provided herein is a method of treating a thromboembolic disorder in a subject in need thereof, the method comprising administering to the subject an effective amount of a pharmaceutical composition described herein, wherein the blood of the subject is contacted with an artificial surface.

In another aspect, provided herein is a method of reducing the risk of a thromboembolic disorder in a subject in need thereof, the method comprising administering to the subject an effective amount of a pharmaceutical composition described herein, wherein the blood of the subject is contacted with an artificial surface.

Also provided herein is a method of prophylaxis of a thromboembolic disorder in a subject in need thereof, the method comprising administering to the subject an effective amount of a pharmaceutical composition described herein, wherein the blood of the subject is contacted with an artificial surface.

In some embodiments of the methods described herein, the artificial surface is in contact with blood in the subject's circulatory system.

In some embodiments, the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus.

In some embodiments, the artificial surface causes or is associated with the thromboembolic disorder.

In some embodiments, the thromboembolic disorder is a venous thromboembolism, deep vein thrombosis, or pulmonary embolism.

In some embodiments, the thromboembolic disorder is a blood clot.

In some embodiments, the methods described herein further comprises conditioning the artificial surface with a separate dose of a pharmaceutical composition described herein prior to contacting the artificial surface with blood in the circulatory system of the subject.

In some embodiments, the methods described herein further comprises conditioning the artificial surface with a separate dose of a pharmaceutical composition described herein prior to or during administration of the pharmaceutical composition to the subject.

In some embodiments, the methods described herein further comprises conditioning the artificial surface with a separate dose of a pharmaceutical composition described herein prior to and during administration of the pharmaceutical composition to the subject.

In some embodiments of the methods described herein, the artificial surface is a cardiopulmonary bypass circuit.

In some embodiments of the methods described herein, the artificial surface is an extracorporeal membrane oxygenation (ECMO) apparatus. In some embodiments, the ECMO apparatus is venovenous ECMO apparatus or venoarterial ECMO apparatus.

In another aspect, disclosed herein is a method of preventing or reducing a risk of a thromboembolic disorder in a subject during or after a medical procedure, comprising:

(i) administering to the subject an effective amount of a pharmaceutical composition described herein, before, during, or after the medical procedure; and

(ii) contacting blood of the subject with an artificial surface;

thereby preventing or reducing the risk of the thromboembolic disorder during or after the medical procedure.

In some embodiments, the artificial surface is conditioned with a pharmaceutical composition described herein prior to administration of the pharmaceutical composition to the subject prior to, during, or after the medical procedure.

In some embodiments, the pharmaceutical composition for conditioning the artificial surface further comprises a solution, wherein the solution is selected from the group consisting of a saline solution, Ringer's solution, and blood.

In some embodiments, the thromboembolic disorder is a blood clot.

In some embodiments, the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

In some embodiments, the medical procedure comprises a cardiopulmonary bypass.

In some embodiments, the medical procedure comprises an oxygenation and pumping of blood via extracorporeal membrane oxygenation (ECMO). In some embodiments, the ECMO is venovenous ECMO or venoarterial ECMO.

In some embodiments of the methods described herein, the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

In another aspect, provided herein is a method of treating the blood of a subject in need thereof, the method comprising administering to the subject an effective amount of a pharmaceutical composition described herein.

In some embodiments of the methods described herein, the pharmaceutical composition is administered to the subject intravenously. In other embodiments of the methods described herein, the pharmaceutical composition is administered to the subject subcutaneously. In some embodiments, the pharmaceutical composition is administered to the subject as a continuous intravenous infusion. In some embodiments, the pharmaceutical composition is administered to the subject as a bolus.

In some embodiments, the subject is a human. In some embodiments, the subject has an elevated risk of a thromboembolic disorder. In some embodiments, the thromboembolic disorder is a result of a complication in surgery. In some embodiments, the subject is sensitive to or has developed sensitivity to heparin. In some embodiments, the subject is resistant to or has developed resistance to heparin.

### Pharmaceutical Compositions

The compositions described herein include the compound described herein (e.g., Compound 1 or a pharmaceutically acceptable salt thereof) as well as additional therapeutic agents, if present, in amounts effective for achieving the treatment of a disease or disease symptoms (e.g., such as a disease associated with Factor XIa or kallikrein).

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions may be in the form of a solid composition (e.g., lyophilized composition) that can be reconstituted by addition of a compatible reconstitution diluent prior to parenteral administration or in the form of a frozen composition adapted to be thaws and, if desired, diluted with a compatible diluent prior to parenteral administration. In some embodiments, the pharmaceutical composition includes particles or a powder (e.g., lyophilized composition) dissolved in an aqueous medium, (e.g., a saline solution, dextrose solution) in a unit dosage IV bag or bottle at a concentration suitable for intravenous administration to a subject. In some embodiments, ingredients of a pharmaceutical composition suitable for intravenous administration are separated from each other in a single container, e.g., a powder comprising a compound described herein or a pharmaceutically acceptable salt thereof, is separated from an aqueous medium such as a saline solution. In this latter example, the various components are separated by a seal that can be broken to contact the ingredients with each other to form the pharmaceutical composition suitable for intravenous administration.

In an aspect, provided herein is an aqueous pharmaceutical composition comprising a compound of Formula (I-A)

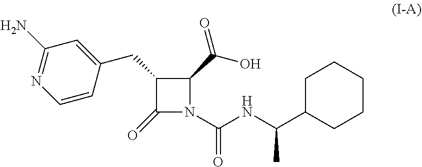 [SMILES: C[C@@H](NC(=O)N1C(=O)[C@H](Cc2ccnc(N)c2)[C@H]1C(=O)O)C1CCCCC1]

or a pharmaceutically acceptable salt thereof, a cyclodextrin, and an excipient.

In some embodiments, the pharmaceutical composition comprises the compound of Formula (I-A), the cyclodextrin, and the excipient. In some embodiments, the cyclodextrin is selected from the group consisting of alkyl cyclodextrin, hydroxyalkyl cyclodextrin, carboxyalkyl cyclodextrin, and sulfoalkyl ether cyclodextrin. In some embodiments, the cyclodextrin is hydroxypropyl β-cyclodextrin. In some embodiments, the cyclodextrin is sulfobutyl ether β-cyclodextrin.

In some embodiments, the excipient is a sugar (e.g., a saccharide (e.g., monosaccharide, disaccharide, or polysaccharide)) or a sugar alcohol. For example, the excipient is sucrose, lactose, trehalose, dextran, erythritol, arabitol, xylitol, sorbitol, or mannitol, or a combination thereof. In some embodiments, the excipient is mannitol. In some embodiments, the excipient is lactose.

In some embodiments, the pharmaceutical composition described herein further comprises a buffer. In some embodiments, the buffer is a monoprotic acid or a polyprotic acid or a combination thereof. In some embodiments, the buffer is a solution of one or more substances. In some embodiments, the buffer is a solution of a salt of a weak acid and a weak base. In some embodiments, the buffer is a solution of a salt of the weak acid with a strong base. In some embodiments, the buffer is selected from the group consisting of a maleate buffer, a citrate buffer, and a phosphate buffer. In some embodiments, the buffer is a phosphate buffer. In some embodiments, the phosphate buffer is a solution of monosodium phosphate, disodium phosphate, trisodium phosphate, or a combination thereof.

In some embodiments, the pharmaceutical composition further comprises a solubilizing agent. In some embodiments, the solubilizing agent is a polyoxyethylene sorbitan ester (e.g, TWEEN® 20) or a polyethylene glycol (e.g., PEG400).

In some embodiments, the solubilizing agent is in an amount of from about 0.01% to about 1%, about 0.01% to about 0.9%, about 0.01% to about 0.8%, about 0.01% to about 0.7%, about 0.01% to about 0.6, about 0.01% to about 0.5%, about 0.01% to about 0.4%, about 0.01% to about 0.3%, about 0.01% to about 0.2%, about 0.01% to about 0.1%, or about 0.01% to about 0.05% by weight relative to weight of the compound of Formula (I-A).

In some embodiments, the pH of the composition is from about 2 to about 8 (e.g., from about 3 to about 7, from about 4 to about 7, from about 5 to about 6, from about 6 to about 7, from about 6 to about 8, from about 5 to about 8, from about 4 to about 8, or from about 3 to about 8). In some embodiments, the pH is from about 6 to about 8. In some embodiments, the pH is about 6 to about 7. In some embodiments, the pH is about 7. In some embodiments, the pH is about 6.8.

In some embodiments, the concentration of the compound of Formula (I-A) is from about 0.1 mg/mL to about 100 mg/mL, about 0.1 mg/mL to about 80 mg/mL, about 0.1 mg/mL to about 60 mg/mL, about 0.1 mg/mL to about 40 mg/mL, about 0.1 mg/mL to about 20 mg/mL, about 0.1 mg/mL to about 10 mg/mL, about 1 mg/mL to about 100 mg/mL, about 1 mg/mL to about 80 mg/mL, about 1 mg/mL to about 60 mg/mL, about 1 mg/mL to about 40 mg/mL, about 1 mg/mL to about 20 mg/mL, about 1 mg/mL to about 10 mg/mL, about 10 mg/mL to about 100 mg/mL, about 10 mg/mL to about 80 mg/mL, about 10 mg/mL to about 60 mg/mL, about 10 mg/mL to about 40 mg/mL, about 20 mg/mL to about 100 mg/mL, about 20 mg/mL to about 80 mg/mL, about 20 mg/mL to about 60 mg/mL, about 40 mg/mL to about 100 mg/mL, about 40 mg/mL to about 80 mg/mL, about 60 mg/mL to about 100 mg/mL, about 60 mg/mL to about 80 mg/mL, or about 80 mg/mL to about 100 mg/mL.

In some embodiments, the concentration of the compound of Formula (I-A) is about 0.1 mg/mL, about 1 mg/mL, about 2.5 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL. In some embodiments, the concentration of the compound of Formula (I-A) is about 10 mg/mL. In some embodiments, the concentration of the compound of Formula (I-A) is about 3 mg/mL. In some embodiments, the concentration of the compound of Formula (I-A) is about 1 mg/mL.

In some embodiments, the concentration of the buffer is from about 1 mM to about 500 mM, about 1 mM to about 250 mM, about 1 mM to about 100 mM, about 1 mM to about 50 mM, about 1 mM to about 20 mM, about 1 mM to about 10 mM, 10 mM to about 500 mM, about 10 mM to about 250 mM, about 10 mM to about 100 mM, about 10 mM to about 50 mM, about 10 mM to about 20 mM, about 20 mM to about 500 mM, about 20 mM to about 250 mM, about 20 mM to about 100 mM, about 20 mM to about 50 mM, about 50 mM to about 500 mM, about 50 mM to about 250 mM, about 50 mM to about 100 mM, about 100 mM to about 500 mM, or about 100 mM to about 250 mM.

In some embodiments, the concentration of the buffer is about 5 mM, about 10 mM, about 15 mM, about 20 mM, about 25 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 300 mM, about 350 mM, about 400 mM, about 450 mM, or about 500 mM. In some embodiments, the concentration of the buffer is about 10 mM.

In some embodiments, the buffer is a phosphate buffer.

In some embodiments, the concentration of the phosphate buffer is from about 1 mM to about 500 mM, about 1 mM to about 250 mM, about 1 mM to about 100 mM, about 1 mM to about 50 mM, about 1 mM to about 20 mM, about 1 mM to about 10 mM, 10 mM to about 500 mM, about 10 mM to about 250 mM, about 10 mM to about 100 mM, about 10 mM to about 50 mM, about 10 mM to about 20 mM, about 20 mM to about 500 mM, about 20 mM to about 250 mM, about 20 mM to about 100 mM, about 20 mM to about 50 mM, about 50 mM to about 500 mM, about 50 mM to about 250 mM, about 50 mM to about 100 mM, about 100 mM to about 500 mM, or about 100 mM to about 250 mM.

In some embodiments, the concentration of the phosphate buffer is about 5 mM, about 10 mM, about 15 mM, about 20 mM, about 25 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 300 mM, about 350 mM, about 400 mM, about 450 mM, or about 500 mM. In some embodiments, the concentration of the phosphate buffer is about 10 mM.

In some embodiments, the cyclodextrin is in an amount of from about 0.1% to about 10%, about 0.1% to about 7.5%, about 0.1% to about 5%, about 0.1% to about 3.5%, about 0.1% to about 1%, about 1% to about 10%, about 1% to about 7.5%, about 1% to about 5%, about 3% to about 10%, about 3% to about 7.5%, or about 3% to about 5% by weight relative to weight of the compound of Formula (I-A). In some embodiments, the cyclodextrin is in an amount of about 1%, 1.5%, 2%, 2.5%, 3%, 3.5%, 4%, 4.5% or 5% by weight relative to weight of the compound of Formula (I-A). In some embodiments, the cyclodextrin is in an amount of from about 0.1% to about 10% (e.g., about 0.5% to about 6% (e.g., about 0.7% to about 5.6% (e.g., about 2.1% to about 5%))) by weight relative to weight of the compound of Formula (I-A). In some embodiments, the cyclodextrin is in an amount of about 3.5% by weight relative to weight of the compound of Formula (I-A). In some embodiments, the cyclodextrin is in an amount of about 5% by weight relative to weight of the compound of Formula (I-A).

In some embodiments, the cyclodextrin is hydroxypropyl β-cyclodextrin.

In some embodiments, the excipient is in an amount of from about 0.1% to about 10%, about 0.1% to about 7.5%, about 0.1% to about 5%, about 0.1% to about 3.5%, about 0.1% to about 1%, about 1% to about 30%, about 1% to about 20%, about 1% to about 10%, about 1% to about 7.5%, about 1% to about 5%, about 3% to about 10%, about 3% to about 7.5%, about 3% to about 5%, about 3% to about 20%, about 3% to about 30%, about 5% to about 20%, or about 5% to about 30% by weight relative to weight of the compound of Formula (I-A). In some embodiments, the excipient is in an amount of about 1%, 1.5%, 2%, 2.5%, 3%, 3.5%, 4%, 4.5%, 5%, 10%, 20%, or 30% by weight relative to weight of the compound of Formula (I-A). In some embodiments, the excipient is in an amount of about 3% by weight relative to weight of the compound of Formula (I-A). In some embodiments, the excipient is an amount of about 5% by weight relative to weight of the compound of Formula (I-A).

In some embodiments, the excipient agent is mannitol. In some embodiments, the excipient is lactose.

In another aspect, provided herein is a lyophilized formulation comprising of a composition which prior to lyophilization corresponds to an aqueous pharmaceutical composition described herein (e.g., an aqueous pharmaceutical composition comprising a compound of Formula (I-A) or a pharmaceutically acceptable salt thereof, a cyclodextrin, and an excipient). In some embodiments, the lyophilized formulation as described herein is reconstituted in an aqueous medium, thereby preparing an aqueous pharmaceutical solution suitable for parenteral administration to a subject in need thereof.

In another aspect, provided herein is a pharmaceutical composition comprising particles, wherein the particles comprise a compound of Formula (I-A)

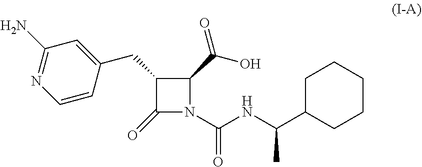 [SMILES: C[C@@H](NC(=O)N1C(=O)[C@H](Cc2ccnc(N)c2)[C@H]1C(=O)O)C1CCCCC1]

or a pharmaceutically acceptable salt thereof, a cyclodextrin, and a bulking agent.

In some embodiments, the pharmaceutical composition comprises the compound of Formula (I-A), the cyclodextrin, and the bulking agent. In some embodiments, the cyclodextrin is selected from the group consisting of alkyl cyclodextrin, hydroxyalkyl cyclodextrin, carboxyalkyl cyclodextrin, and sulfoalkyl ether cyclodextrin. In some embodiments, the cyclodextrin is hydroxypropyl β-cyclodextrin. In some embodiments, the cyclodextrin is sulfobutyl ether β-cyclodextrin.

In some embodiments, the bulking agent is a sugar (e.g., a saccharide (e.g., monosaccharide, disaccharide, or polysaccharide)) or a sugar alcohol. In some embodiments, the bulking agent is sucrose, lactose, trehalose, dextran, erythritol, arabitol, xylitol, sorbitol, or mannitol, or a combination thereof. In some embodiments, the bulking agent is mannitol. In some embodiments, the bulking agent is lactose.

In some embodiments, the bulking agent is a lyoprotectant.

In some embodiments, the concentration of the compound of Formula (I-A) is from about 0.1% to about 10%, about 0.1% to about 7.5%, about 0.1% to about 5%, about 0.1% to about 3.5%, about 0.1% to about 1%, about 1% to about 10%, about 1% to about 7.5%, about 1% to about 5%, about 3% to about 10%, about 3% to about 7.5%, or about 3% to about 5% by weight of the composition. In some embodiments, the concentration of the compound of Formula (I-A) is about 1%, 2%, 3%, 4%, 5%, 6%, 7%, 8%, 9%, or 10% by weight of the composition. In some embodiments, the concentration of the compound of Formula (I-A) is about 1% by weight of the composition. In some embodiments, the concentration of the compound of Formula (I-A) is about 0.3% by weight of the composition.

In some embodiments, the cyclodextrin is in an amount of from about 0.1% to about 10%, about 0.1% to about 7.5%, about 0.1% to about 5%, about 0.1% to about 3.5%, about 0.1% to about 1%, about 1% to about 10%, about 1% to about 7.5%, about 1% to about 5%, about 3% to about 10%, about 3% to about 7.5%, or about 3% to about 5% by weight relative to weight of the compound of Formula (I-A). In some embodiments, the cyclodextrin is in an amount of about 1%, 1.5%, 2%, 2.5%, 3%, 3.5%, 4%, 4.5% or 5% by weight relative to weight of the compound of Formula (I-A). In some embodiments, the cyclodextrin is in an amount of from about 0.1% to about 10% (e.g., about 0.5% to about 6% (e.g., about 0.7% to about 5.6% (e.g., about 2.1% to about 5%))) by weight relative to weight of the compound of Formula (I-A). In some embodiments, the cyclodextrin is in an amount of about 3.5% by weight relative to weight of the compound of Formula (I-A). In some embodiments, the cyclodextrin is in an amount of about 5% by weight relative to weight of the compound of Formula (I-A).

In some embodiments, the cyclodextrin is hydroxypropyl β-cyclodextrin.

In some embodiments, the excipient is in an amount of from about 0.1% to about 10%, about 0.1% to about 7.5%, about 0.1% to about 5%, about 0.1% to about 3.5%, about 0.1% to about 1%, about 1% to about 30%, about 1% to about 20%, about 1% to about 10%, about 1% to about 7.5%, about 1% to about 5%, about 3% to about 10%, about 3% to about 7.5%, about 3% to about 5%, about 3% to about 20%, about 3% to about 30%, about 5% to about 20%, or about 5% to about 30% by weight relative to weight of the compound of Formula (I-A). In some embodiments, the excipient is in an amount of about 1%, 1.5%, 2%, 2.5%, 3%, 3.5%, 4%, 4.5%, 5%, 10%, 20%, or 30% by weight relative to weight of the compound of Formula (I-A). In some embodiments, the excipient is in an amount of about 3% by weight relative to weight of the compound of Formula (I-A). In some embodiments, the excipient is an amount of about 5% by weight relative to weight of the compound of Formula (I-A).

In some embodiments, the excipient agent is mannitol. In some embodiments, the excipient is lactose.

In another aspect, provided herein is a process for preparing an aqueous pharmaceutical composition from the pharmaceutical composition described herein (e.g., a pharmaceutical composition comprising particles, wherein the particles comprise a compound of Formula (I-A) or a pharmaceutically acceptable salt thereof, a cyclodextrin, and a bulking agent), the process comprising reconstituting the pharmaceutical composition into an aqueous medium, thereby forming the aqueous composition. In some embodiments, the aqueous medium is deionized water. In some embodiments, the aqueous medium comprises sodium chloride. In some embodiments, the aqueous medium comprises about 5% dextrose.

In some embodiments, the composition is prepared to be suitable for parenteral administration to a subject in need thereof. In some embodiments, the composition is prepared to be suitable for intramuscular, subcutaneous or intravenous administration to a subject in need thereof.

In some embodiments, the pH of the reconstituted composition is from about 2 to about 8 (e.g., from about 3 to about 7, from about 4 to about 7, from about 5 to about 6, from about 6 to about 7, from about 6 to about 8, from about 5 to about 8, from about 4 to about 8, or from about 3 to about 8). In some embodiments, the pH of the reconstituted composition is from about 6 to about 8. In some embodiments, the pH of the reconstituted composition is about 6 to about 7. In some embodiments, the pH of the reconstituted composition is about 7. In some embodiments, the pH of the reconstituted composition is about 6.8.

In some embodiments, the concentration of the compound of Formula (I-A) in the reconstituted composition is from about 0.01 mg/mL to about 100 mg/mL, about 0.01 mg/mL to about 50 mg/mL, about 0.01 mg/mL to about 10 mg/mL, about 0.01 mg/mL to about 1 mg/mL, about 0.01 mg/mL to about 0.1 mg/mL, about 0.1 mg/mL to about 100 mg/mL, about 0.1 mg/mL to about 80 mg/mL, about 0.1 mg/mL to about 60 mg/mL, about 0.1 mg/mL to about 40 mg/mL, about 0.1 mg/mL to about 20 mg/mL, about 0.1 mg/mL to about 10 mg/mL, about 1 mg/mL to about 100 mg/mL, about 1 mg/mL to about 80 mg/mL, about 1 mg/mL to about 60 mg/mL, about 1 mg/mL to about 40 mg/mL, about 1 mg/mL to about 20 mg/mL, about 1 mg/mL to about 10 mg/mL, about 10 mg/mL to about 100 mg/mL, about 10 mg/mL to about 80 mg/mL, about 10 mg/mL to about 60 mg/mL, about 10 mg/mL to about 40 mg/mL, about 20 mg/mL to about 100 mg/mL, about 20 mg/mL to about 80 mg/mL, about 20 mg/mL to about 60 mg/mL, about 40 mg/mL to about 100 mg/mL, about 40 mg/mL to about 80 mg/mL, about 60 mg/mL to about 100 mg/mL, about 60 mg/mL to about 80 mg/mL, or about 80 mg/mL to about 100 mg/mL.

In some embodiments, the concentration of the compound of Formula (I-A) in the reconstituted formulation is about 0.01 mg/mL, 0.03 mg/mL, 0.05 mg/mL, 0.1 mg/mL, 0.3 mg/mL, 0.5 mg/mL, about 1 mg/mL, about 2.5 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL. In some embodiments, the concentration of the compound of Formula (I-A) is about 10 mg/mL. In some embodiments, the concentration of the compound of Formula (I-A) is about 1 mg/mL. In some embodiments, the concentration of the compound of Formula (I-A) is about 0.1 mg/mL. In some embodiments, the concentration of the compound of Formula (I-A) is about 0.3 mg/mL. In some embodiments, the concentration of the compound of Formula (I-A) is about 0.03 mg/mL.

### Routes of Administration

The pharmaceutical compositions provided herewith may be administered orally, rectally, or parenterally (e.g., intravenous infusion, intravenous bolus injection, inhalation, implantation). The term parenteral as used herein includes subcutaneous, intracutaneous, intravenous (e.g., intravenous infusion, intravenous bolus injection), intranasal, inhalation, pulmonary, transdermal, intramuscular, intraarticular, intraarterial, intrasynovial, intrasternal, intrathecal, intralesional and intracranial injection or other infusion techniques. The pharmaceutical compositions provided herewith may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The pharmaceutical compositions may be in the form of a sterile injectable preparation, for example, as a sterile injectable aqueous or oleaginous solution or suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans or other similar emulsifying agents or bioavailability enhancers which are commonly used in the manufacture of pharmaceutically acceptable solid, liquid, or other dosage forms may also be used for the purposes of formulation. In some embodiments, the intravenous pharmaceutical composition comprises a carrier selected from the group consisting of 5% w/w dextrose water (“5DW”) and saline.

The pharmaceutical compositions provided herewith may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions or emulsions are administered orally, the active ingredient may be suspended or dissolved in an oily phase is combined with emulsifying or suspending agents. If desired, certain sweetening or flavoring or coloring or taste masking agents may be added.

The compounds described herein can, for example, be administered by injection, intravenously (e.g., intravenous infusion, intravenous bolus injection), intraarterially, subdermally, intraperitoneally, intramuscularly, or subcutaneously; or orally, buccally, nasally, transmucosally, topically with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day (e.g., by intravenous bolus injection) or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

In some embodiments, the compound or pharmaceutical composition is administered to the subject intravenously. In some embodiments, the compound or pharmaceutical composition is administered to the subject subcutaneously. In some embodiments, the compound or pharmaceutical composition is administered to the subject as a continuous intravenous infusion. In some embodiments, the compound or pharmaceutical composition is administered to the subject as a bolus. In some embodiments, the compound or pharmaceutical composition is administered to the subject as a bolus followed by a continuous intravenous infusion.

In some embodiments, a pharmaceutical composition formulated for subcutaneous administration or intravenous administration is administered to a subject from 1 time per day to 6 times per day (e.g., 1 time per day, 2 times per day, or 4 times per day).

### Combinations

In carrying out the methods of the present invention, it may be desired to administer the compounds of the invention (e.g., Factor XIa or kallikrein inhibitors) in combination with each other and one or more other agents for achieving a therapeutic benefit such as antithrombotic or anticoagulant agents, anti-hypertensive agents, anti-ischemic agents, anti-arrhythmic agents, platelet function inhibitors, and so forth. For example, the methods of the present invention may be carried out by administering the small molecule Factor XIa or kallikrein inhibitors in combination with a small molecule Factor XIa or kallikrein inhibitor. More particularly, the inventive methods may be carried out by administering the small molecule Factor XIa or kallikrein inhibitors in combination with aspirin, clopidogrel, ticlopidine or CS-747, warfarin, low molecular weight heparins (such as LOVENOX), GPIIb/GPIIIa blockers, PAI-1 inhibitors such as XR-330 and T-686, P2Y1 and P2Y12 receptor antagonists; thromboxane receptor antagonists (such as ifetroban), prostacyclin mimetics, thromboxane A synthetase inhibitors (such as picotamide), serotonin-2-receptor antagonists (such as ketanserin); compounds that inhibit other coagulation factors such as FVII, FVIII, FIX, FX, prothrombin, TAFI, and fibrinogen, or other compounds that inhibit FXI or kallikrein; fibrinolytics such as TPA, streptokinase, PAI-1 inhibitors, and inhibitors of □-2-antiplasmin such as anti-□-2-antiplasmin antibody fibrinogen receptor antagonists, inhibitors of □-1-antitrypsin, hypolipidemic agents, such as HMG-CoA reductase inhibitors (e.g., pravastatin, simvastatin, atorvastatin, fluvastatin, cerivastatin, AZ4522, and itavastatin), and microsomal triglyceride transport protein inhibitors (such as disclosed in U.S. Pat. Nos. 5,739,135, 5,712,279 and 5,760,246); antihypertensive agents such as angiotensin-converting enzyme inhibitors (e.g., captopril, lisinopril or fosinopril); angiotensin-II receptor antagonists (e.g., irbesartan, losartan or valsartan); ACE/NEP inhibitors (e.g., omapatrilat and gemopatrilat); or □-blockers (such as propranolol, nadolol and carvedilol). The inventive methods may be carried out by administering the small molecule Factor XIa or kallikrein inhibitors in combination with anti-arrhythmic agents such as for atrial fibrillation, for example, amiodarone or dofetilide. The inventive methods may also be carried out in combination continuous renal replacement therapy for treating, e.g., acute kidney injury.

In carrying out the methods of the present invention, it may be desired to administer the compounds of the invention (Factor XIa or kallikrein inhibitors) in combination with agents that increase the levels of cAMP or cGMP in cells for a therapeutic benefit. For example, the compounds of the invention may have advantageous effects when used in combination with phosphodiesterase inhibitors, including PDE1 inhibitors (such as those described in Journal of Medicinal Chemistry, Vol. 40, pp. 2196-2210 [1997]), PDE2 inhibitors, PDE3 inhibitors (such as revizinone, pimobendan, or olprinone), PDE4 inhibitors (such as rolipram, cilomilast, or piclamilast), PDE7 inhibitors, or other PDE inhibitors such as dipyridamole, cilostazol, sildenafil, denbutyline, theophylline (1,2-dimethylxanthine), ARIFLOT™ (i.e., cis-4-cyano-4-[3-(cyclopentylox-y)-4-methoxyphenyl]cyclohexane-1-carboxyl-ic acid), arofyline, roflumilast, C-11294A, CDC-801, BAY-19-8004, cipamfylline, SCH351591, YM-976, PD-189659, mesiopram, pumafentrine, CDC-998, IC-485, and KW-4490.

The inventive methods may be carried out by administering the compounds of the invention in combination with prothrombolytic agents, such as tissue plasminogen activator (natural or recombinant), streptokinase, reteplase, activase, lanoteplase, urokinase, prourokinase, anisolated streptokinase plasminogen activator complex (ASPAC), animal salivary gland plasminogen activators, and the like.

The inventive methods may be carried out by administering the compounds of the invention in combination with 0-adrenergic agonists such as albuterol, terbutaline, formoterol, salmeterol, bitolterol, pilbuterol, or fenoterol; anticholinergics such as ipratropium bromide; anti-inflammatory cortiocosteroids such as beclomethasone, triamcinolone, budesonide, fluticasone, flunisolide or dexamethasone; and anti-inflammatory agents such as cromolyn, nedocromil, theophylline, zileuton, zafirlukast, monteleukast and pranleukast.

Small molecule Factor XIa or kallikrein inhibitors may act synergistically with one or more of the above agents. Thus, reduced doses of thrombolytic agent(s) may be used, therefore obtaining the benefits of administering these compounds while minimizing potential hemorrhagic and other side effects.

### Course of Treatment

The compositions described herein include an effective amount of a compound of the invention (e.g., a Factor XIa or kallikrein inhibitor) optionally in combination with one or more other agents (e.g., an additional therapeutic agent) such as antithrombotic or anticoagulant agents, anti-hypertensive agents, anti-ischemic agents, anti-arrhythmic agents, platelet function inhibitors, and so forth for achieving a therapeutic benefit.

In some embodiments, the additional therapeutic agent is administered following administration of the composition of the invention. In some embodiments, the additional therapeutic agent is administered 15 minutes, 30 minutes, 1 hour, 2 hours, 4 hours, 6 hours, 8 hours, 10 hours, 12 hours, 14 hours, 18 hours, 24 hours, 48 hours, 72 hours or longer after administration of the composition of the invention. In some embodiments, the additional therapeutic agent is administered (e.g., orally) after discharge from a medical facility (e.g., a hospital).

In some embodiments, the compound of the invention (e.g., a Factor XIa or kallikrein inhibitor) and the additional therapeutic agent are co-formulated into a single composition or dosage. In some embodiments, the compound of the invention (e.g., a Factor XIa or kallikrein inhibitor) and the additional therapeutic agent are administered separately. In some embodiments, the compound of the invention (e.g., a Factor XIa or kallikrein inhibitor) and the additional therapeutic agent are administered sequentially. In some embodiments, the compound of the invention (e.g., a Factor XIa or kallikrein inhibitor) and the additional therapeutic agent are administered separately and sequentially. In general, at least one of the compound of the invention (e.g., a Factor XIa or kallikrein inhibitor) and the additional therapeutic agent is administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally, for example, intramuscular injection or intravenously (e.g., intravenous infusion, intravenous bolus injection)). In some embodiments, compound of the invention is administered parenterally (e.g., intranasally, buccally, intravenously (e.g., intravenous infusion, intravenous bolus injection) or intramuscularly). In some embodiments, the additional therapeutic agent is administered orally. In some embodiments, the compound of the invention (e.g., a Factor XIa or kallikrein inhibitor) is administered parenterally (e.g., intranasally, buccally, intravenously (e.g., intravenous infusion, intravenous bolus injection) or intramuscularly) and the additional therapeutic agent is administered orally.

In some embodiments, the composition of the invention may be administered once or several times a day. A duration of treatment may follow, for example, once per day for a period of about 1, 2, 3, 4, 5, 6, 7 days or more. In some embodiments, the treatment is chronic (e.g., for a lifetime). In some embodiments, either a single dose in the form of an individual dosage unit or several smaller dosage units or by multiple administrations of subdivided dosages at certain intervals is administered. For instance, a dosage unit can be administered from about 0 hours to about 1 hr, about 1 hr to about 24 hr, about 1 to about 72 hours, about 1 to about 120 hours, or about 24 hours to at least about 120 hours post injury. Alternatively, the dosage unit can be administered from about 0.5, 1, 1.5, 2, 3, 4, 5, 6, 7, 8, 9, 10, 11, 12, 13, 14, 15, 16, 17, 18, 19, 20, 21, 22, 23, 24, 30, 40, 48, 72, 96, 120 hours or longer post injury. Subsequent dosage units can be administered any time following the initial administration such that a therapeutic effect is achieved. In some embodiments, the initial dose is administered orally. In some embodiments, doses subsequent to the initial dose are administered parenterally (e.g., intranasally, intramuscularly buccally, inhalation, implantation, transdermal, intravenously (e.g., intravenous infusion, intravenous bolus injection), subcutaneous, intracutaneous, intranasal, pulmonary, transdermal, intraarticular, intraarterial, intrasynovial, intrasternal, intrathecal, intralesional and intracranial injection or other infusion techniques); orally; or rectally.

In some embodiments, composition of the invention is administered orally, e.g., as an liquid or solid dosage form for ingestion, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the composition is administered orally as a liquid dosage form. In another embodiment, the composition is administered orally as a solid dosage form.

Where a subject undergoing therapy exhibits a partial response, or a relapse following completion of the first cycle of the therapy, subsequent courses of therapy may be needed to achieve a partial or complete therapeutic response (e.g., chronic treatment, e.g., for a lifetime).

In some embodiments, the composition described herein is administered intravenously, e.g., as an intravenous infusion or intravenous bolus injection, for about 5 minutes to about 1 week; about 30 minutes to about 24 hours, about 1 hour to about 12 hours, about 2 hours to about 12 hours, about 4 hours to about 12 hours, about 6 hours to about 12 hours, about 6 hours to about 10 hours; about 5 minutes to about 1 hour, about 5 minutes to about 30 minutes; about 12 hours to about 1 week, about 24 hours to about 1 week, about 2 days to about 5 days, or about 3 days to about 5 days. In one embodiment, the composition described herein is administered as an intravenous infusion for about 5, 10, 15, 30, 45, or 60 minutes or longer; about 1, 2, 4, 6, 8, 10, 12, 16, or 24 hours or longer; about 1, 2, 3, 4, 5, 6, 7, 8, 9, or 10 days or longer.

### Dosages and Dosing Regimens

The effective amount of a composition administered according to the present invention may be determined by one of ordinary skill in the art. The specific dose level and frequency of dosage for any particular subject may vary and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

Upon improvement of a patient's condition, a maintenance dose of a composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

### EXAMPLES

In order that the invention described herein may be more fully understood, the following examples are set forth. Starting materials and various intermediates described in the following examples may be obtained from commercial sources, prepared from commercially available organic compounds, or prepared using known synthetic methods. The examples described in this application are offered to illustrate the compounds provided herein and are not to be construed in any way as limiting their scope.

### General Procedures

All non-aqueous reactions were run under an atmosphere of nitrogen to maintain an anhydrous atmosphere and to maximize yields. All reactions were stirred using an overhead stirring assembly or magnetically, with the aid of a Teflon-coated stir bar. The description ‘drying over’ refers to drying of a reaction product solution over a specified drying agent and then filtration of the solution though a suitable filter paper or through a sintered glass funnel. The descriptions ‘was concentrated’, ‘was concentrated at reduced pressure’, or ‘evaporated’ refers to removal of solvents under reduced pressure using a rotary evaporator. Chromatography or chromatographed refers to the use of flash column chromatography on silica gel unless otherwise specified. Flash chromatography refers to column chromatography under gas pressure (for example, nitrogen) or a mechanical pump to apply solvent pressure such as with a commercial system as supplied by Biotage or other vendors. Unless otherwise specified, proton NMR spectra (H) are measured at 400 MHz and carbon NMR spectra (¹³C) are measured at 100 MHz in the specified solvent.

Abbreviations used in the experimental examples are listed in the Abbreviations Table below.

Abbreviation Table

| | |
|---|---|
| MeCN or ACN | Acetonitrile |
| EDC | 1,2-Dichloroethane |
| DCM | Dichloromethane |
| DMAP | 4-Dimethylaminopyridine |
| EA | Ethyl acetate |
| Ether | Diethyl ether |
| hr or h | Hours |
| HPLC | High-performance |
| | liquid chromatography |
| IPA | Isopropyl alcohol |
| min | Minutes |
| TBME | Methyl tert-butyl ether |
| TEA | Triethylamine |
| NMR | Nuclear magnetic |
| | resonance instrument |
| RT | Room temperature |
| TFA | Trifluoroacetic acid |
| THF | Tetrahydrofuran |
| TPP | Target product profile |
| Concentrated or | Concentration of organic solutions |
| concentrated | under reduced pressure and |
| in vacuo | generally with the use of a |
| | rotary evaporator |
| PMB | p-Methoxybenzyl |
| Boc or BOC | Tert-butyloxycarbonyl |

### Example 1. Exemplary Synthesis of Compound 1.HCl

A non-limiting example of the synthesis of (2S,3R)-3-[(2-aminopyridin-4-yl)methyl]-1-{[(1R)-1-cyclohexylethyl]carbamoyl}-4-oxoazetidine-2-carboxylic acid trifluoroacetate (structure 2 below), tert-butyl(4-bromomethyl)pyridin-2-yl](4-methoxybenzyl)carbamate (structure 3 below), and (R)-(1-isocyanatoethyl)cyclohexane (structure 8 below) can be found in U.S. Pat. No. 9,499,532, which is incorporated herein by reference.

### A. Synthesis of Compound 1.HCl from 2

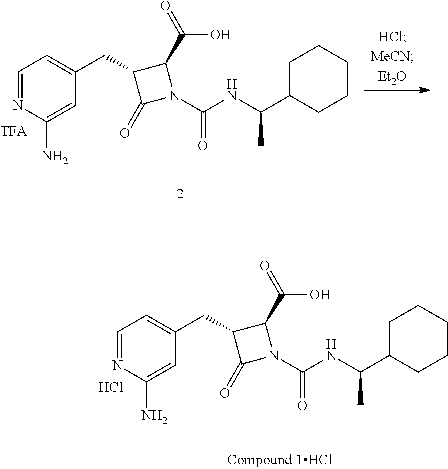 [SMILES: C[C@@H](NC(=O)N1C(=O)[C@H](Cc2ccnc(N)c2)[C@H]1C(=O)O)C1CCCCC1 ; Cl ; C[C@@H](NC(=O)N1C(=O)[C@H](Cc2ccnc(N)c2)[C@H]1C(=O)O)C1CCCCC1]

Acetonitrile (12 mL; 10 vol) was added to (2S,3R)-3-[(2-aminopyridin-4-yl)methyl]-1-{[(1R)-1-cyclohexylethyl]carbamoyl}-4-oxoazetidine-2-carboxylic acid trifluoroacetate (1.23 g; 2.52 mmol) to produce a hazy solution. This mixture was extracted twice with hexane (12 mL); then it was filtered (5 micron) to afford a clear solution. This solution was concentrated to 6 mL (5 volumes) at which point a suspension began to form. Concentrated HCl (0.42 mL; 2 equiv) was added. Then ether (2×12 mL) was added in two portions to induce formation of a precipitate. The mixture was cooled to −1° C. for 15 min. The solids were collected, rinsed with cold ether and air dried to give (2S,3R)-3-[(2-aminopyridin-4-yl)methyl]-1-{[(1R)-1-cyclohexylethyl]carbamoyl}-4-oxoazetidine-2-carboxylic acid hydrochloride 0.82 g (79%) as a white solid.

A highly purified sample was prepared by slurrying the solid in ether (7.5 volumes). The product was collected, rinsed with ether and dried at 50° C. in vacuo overnight.

¹H NMR (400 MHz, CD₃OD) ppm δ 7.79 (1H, d, J=6.8 Hz), 6.99 (1H, s), 6.90 (1H, dd, J=1.5, 6.8 Hz), 6.61 (1H, d J=8.8), 4.28 (1H, d, J=2.8) 3.70 (2H, m), 3.23 (2H, m) 1.75 (5H, m) 1.40 (1H, m) 1.25 (3H, m) 1.15 (3H, d, J=6.8 Hz) 1.00 (2H, m).

HPLC retention time: 3.21 min. HPLC conditions: Column, Zorbax 50 mm; flow=1.5 mL/min; 240 nm; temp=at 30° C.; Solvent A=1 mL TFA/1 L water; Solvent B=2.8 mL TFA/4 L MeCN; Gradient elution sequence: time=0, A:B=95:5; linear gradient to 2:98 A:B over 6 min; linear gradient back to A:B=95:5 from 1 min.

### B. Synthesis of Compound 1.HCl from 3

### Step 1. Preparation of 4: (2S,3R)-3-{2-[(tert-butoxycarbonyl)(4-methoxybenzyl) amino]pyridin-4-yl)methyl)-1-(tert-butyl(dimethyl)silyl]-4-oxoazetidine-2-carboxylic acid

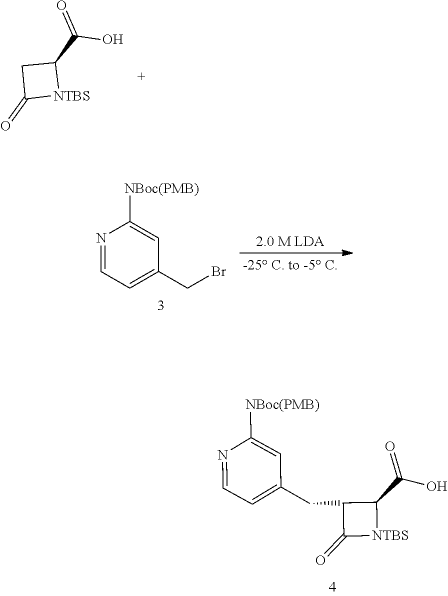

A solution of (2S)-1-tert-butyl(dimethyl)silyl-4-oxoazetidine-2-carboxylic acid (175 g, 0.763 mol) and THE (2 L) was cooled to −25° C. (internal temperature). 2M LDA solution in THE (800 mL, 2.1 eq.) was added dropwise while maintaining the temperature below −10° C. The reaction was stirred for 30 min and a gel-like suspension formed. A solution of tert-butyl(4-bromomethyl)pyridin-2-yl](4-methoxybenzyl)carbamate (342 g, 0.84 mol, 1.12 eq., structure 3) in THF (600 mL) was added dropwise while maintaining the reaction below −5° C. over 2 hr, and then stirred 30 min longer. The reaction was quenched with 1M aqueous KHSO₄ (2 L). The layers were separated and the aqueous layer was extracted with EA (2 L×2). The combined organic phase was washed with brine (1 L×2), dried (MgSO₄), filtered and concentrated to give (2S,3R)-3-{2-[(tert-butoxycarbonyl)(4-methoxybenzyl)amino]pyridin-4-yl)methyl)-1-(tert-butyl(dimethyl)silyl]-4-oxoazetidine-2-carboxylic acid as an oily product which was used without purification (436 g, ˜70% purity).

### Step 2. Preparation of compound 5: 4-Methoxybenzyl(2S,3R)-3-({2-[(tert-butoxycarbonyl)(4-methoxybenzyl)amino]pyridin-4-yl}methyl)-1-[(tert-butyl(dimethyl)silyl]-4-oxoazetidine-2-carboxylate

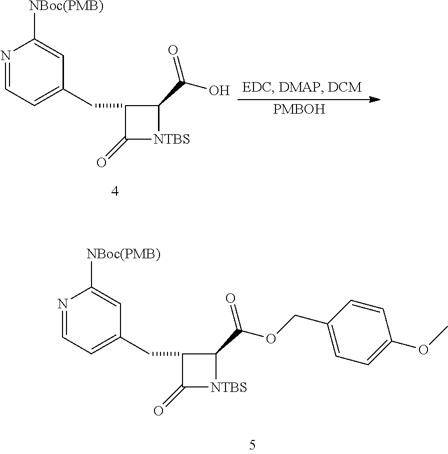

The crude (2S,3R)-3-{2-[(tert-butoxycarbonyl)(4-methoxybenzyl)amino]pyridin-4-yl)methyl)-1-(tert-butyl(dimethyl)silyl]-4-oxoazetidine-2-carboxylic acid was dissolved in DCM (2.5 L) and EDC (137 g, 0.714 mol, 1.3 eq.), PMBOH (76.2 g, 0.55 mol, 1 eq. based on 70% purity of the acid reagent) and DMAP (3.4 g, 0.05 eq.). The solution was stirred at overnight at RT. The mixture was extracted with water (500 ml) and brine (500 ml), dried (MgSO₄), and concentrated. The crude oily residue was chromatographed (gradient elution with 0% to 50% EA/hexanes) to give 4-methoxybenzyl (2S,3R)-3-({2-[(tert-butoxycarbonyl)(4-methoxybenzyl)amino]pyridin-4-yl}methyl)-1-[(tert-butyl(dimethyl)silyl]-4-oxoazetidine-2-carboxylate as a colorless oil (250 g, 48% yield over two steps).

1H NMR (400 MHz, CDCl₃) ppm δ 8.24 (1H, d, J=5.5 Hz), 7.55 (1H, s), 7.22 (2H, d, J=8.8 Hz), 7.20 (2H, d, J=8.8 Hz), 6.89 (1H, dd, J=1.4, 5.2 Hz), 6.87 (2H, d, J=8.6 Hz), 6.79 (2H, d, J=8.6 Hz), 5.09 (2H, s), 5.06 (2H, s), 3.81 (3H, s), 3.77 (1H, d, J=3.3 Hz), 3.76 (3H, s), 3.53 (1H, m), 3.06 (1H, dd, J=0.6, 14.6 Hz), 2.99 (1H, dd, J=7.6, 14.6 Hz), 1.41 (9H, s), 0.82 (9H, s), 0.19 (3H, s), −0.05 (3H, s).

### Step 3. Preparation of compound 6: 4-Methoxybenzyl(2S,3R)-3-({2-[(tert-butoxycarbonyl)(4-methoxybenzyl)amino]pyridin-4-yl}methyl)-4-oxoazetidine-2-carboxylate

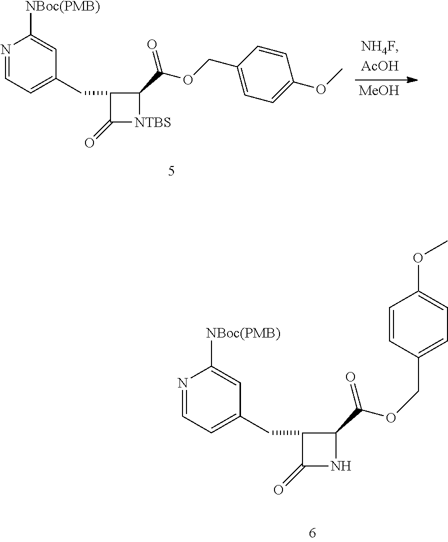

To a solution of 4-methoxybenzyl(2S,3R)-3-({2-[(tert-butoxycarbonyl)(4-methoxybenzyl)amino]pyridin-4-yl}methyl)-1-[(tert-butyl(dimethyl)silyl]-4-oxoazetidine-2-carboxylate (314 g, 0.465 mol) and methanol (1.5 L) was added first acetic acid (112 g, 1.87 mol) and then NH₄F (20.6 g, 0.556 mol, pre-dissolved in 1.2 L of methanol). The mixture was stirred 2 hr at RT. The reaction was concentrated. The residue was dissolved in EA (2 L) and saturated aqueous NaHCO₃ (2 L) was added. The phases were separated and the organic phase was dried (MgSO₄), and concentrated. The oily residue was chromatographed (gradient elution with 0% to 40% EA/hexanes) to give a clear oil which was crystallized from EA/hexanes (1:5) to give 4-methoxybenzyl(2S,3R)-3-({2-[(tert-butoxycarbonyl)(4-methoxybenzyl)amino]pyridin-4-yl}methyl)-4-oxoazetidine-2-carboxylate as a white solid (200 g, 77% yield).

1H NMR (400 MHz, CDCl₃) ppm δ 8.26 (1H, d, J=5.0 Hz), 7.55 (1H, s), 7.21 (2H, d, J=8.6 Hz), 7.19 (2H, d, J=8.6 Hz), 6.91 (1H, dd, J=1.5, 5.0 Hz), 6.88 (2H, d, J=8.8 Hz), 6.79 (2H, d, J=8.8 Hz), 5.92 (1H, s), 5.10 (2H, s), 5.06 (2H, s), 3.87 (1H, d, J=2.5), 3.81 (3H, s), 3.76 (3H, s), 3.56 (1H, m), 3.14 (1H, dd, J=5.8, 14.6 Hz), 3.03 (1H, dd, J=8.1, 14.6 Hz), 1.42 (9H, s).

### Step 4 Preparation of 7: 4-Methoxybenzyl(2S,3R)-3-((2-[(tert-butoxycarbonyl)(4-methoxybenzyl)amino]pyridin-4-yl}methyl)-1-([(1R)-1-cyclohexylethyl)carbamoyl}-4-oxoazetidine-2-carboxylate

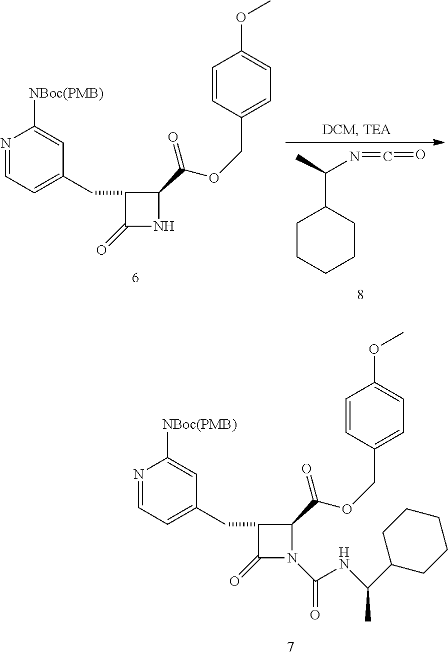

To a solution of 4-methoxybenzyl(2S,3R)-3-({2-[(tert-butoxycarbonyl)(4-methoxybenzyl)amino]pyridin-4-yl}methyl)-4-oxoazetidine-2-carboxylate (210 g, 0.374 mol) and DCM (3 L) was added TEA (188 g, 1.86 mol, 5 eq.) and (R)-(1-isocyanatoethyl)cyclohexane (143 g, 0.933 mol, 2.5 eq, structure 8). The mixture was stirred overnight at RT. The reaction was concentrated. The residue was chromatographed (gradient elution with 0% to 40% EA/hexanes) to give 4-methoxybenzyl-(2S,3R)-3-((2-[(tert-butoxycarbonyl)(4-methoxy benzyl)amino]pyridin-4-yl}methyl)-1-([(1R)-1-cyclohexylethyl)carbamoyl}-4-oxoazetidine-2-carboxylate (159 g, 60% yield) as a white foam.

¹H NMR (400 MHz, CDCl₃) ppm δ 8.25 (1H, d, J=5.1 Hz), 7.61 (1H, s), 7.23 (2H, d, J=8.8 Hz), 7.15 (2H, d, J=8.6 Hz), 6.85 (2H, d, J=8.8 Hz), 6.80 (2H, d J=8.6 Hz), 6.23 (1H, d, J=9.1 Hz), 5.12 (1H, d, J=15.9), 5.11 (2H, s), 5.04 (1H, d, J=12.1 Hz), 4.23 (1H, d, J=2.8 Hz), 3.80 (3H, s), 3.78 (1H, m), 3.76 (3H, s), 3.45 (1H, m), 3.15 (1H, dd, J=6.5, 14.8 Hz), 3.01 (1H, dd, J=8.9, 14.8 Hz), 1.74 (4H, m), 1.68 (2H, m), 1.41 (9H, s), 1.35 (1H, m), 1.21 (2H, m), 1.14 (3H, d, J=6.8 Hz), 0.98 (2H, m).

### Step 5. Preparation of Compound 1.HCl

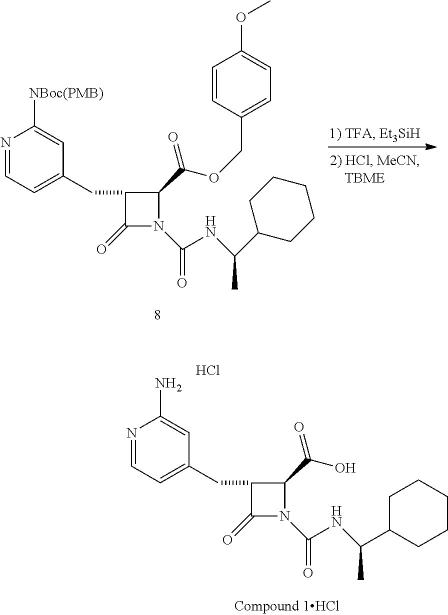

Trifluoroacetic acid (2.1 L) was added to 4-methoxybenzyl-(2S,3R)-3-((2-[(tert-butoxycarbonyl)(4-methoxybenzyl)amino]pyridin-4-yl}methyl)-1-([(1R)-1-cyclohexylethyl)carbamoyl}-4-oxoazetidine-2-carboxylate (283 g, 0.396 mol) giving a red solution. Et₃SiH (138 g, 1.18 mol, 3 eq.) was added and the solution became colorless. The reaction was stirred 4 hr at RT. The TFA was removed in vacuo overnight to give (2S,3R)-3-[(2-aminopyridin-4-yl)methyl]-1-([(1R)-1-cyclohexylethyl]carbamoyl}-4-oxoazetidine-2-carboxylic acid trifluoroacetate as a white foam.

Acetonitrile (1.8 L) was added to the crude TFA salt giving a hazy solution. The solution was clarified by filtration and the residue was washed with acetonitrile (100 mL). The combined acetonitrile solution was extracted with hexanes (1.8 L×3). The acetonitrile solution was concentrated at reduced pressure to about 900 mL. Concentrated HCl (66 mL, 0.792 mol, 2 eq.) was added slowly to form a suspension. TBME (3 L) was added slowly while stirring. The resulting suspension was cooled to 0° C. for 30 min. The solid precipitate was isolated by filtration and rinsed with TBME. The solid was air dried overnight and then dried at 50° C. in vacuo for 5 hr to give (2S,3R)-3 [(2-aminopyridin-4-yl)methyl]-1-{[(1R)-1-cyclohexylethyl]carbamoyl}-4-oxoazetidine-2-carboxylic acid hydrochloride as a white powder (130 g, 80% yield).

¹H NMR (400 MHz, D₂O) ppm δ 7.64 (1H, d, J=6.8 Hz), 6.84 (1H, s), 6.74 (1H, dd, J=1.5, 6.8 Hz), 4.22 (1H, d, J=2.8 Hz), 3.75 (1H, m), 3.54 (1H, m), 3.17 (2H, m), 1.58 (5H, m), 1.22 (1H, m), 1.07 (6H, m), 0.89 (2H, m).

### Example 2. HPLC Method Parameters for the Analysis of Compound 1

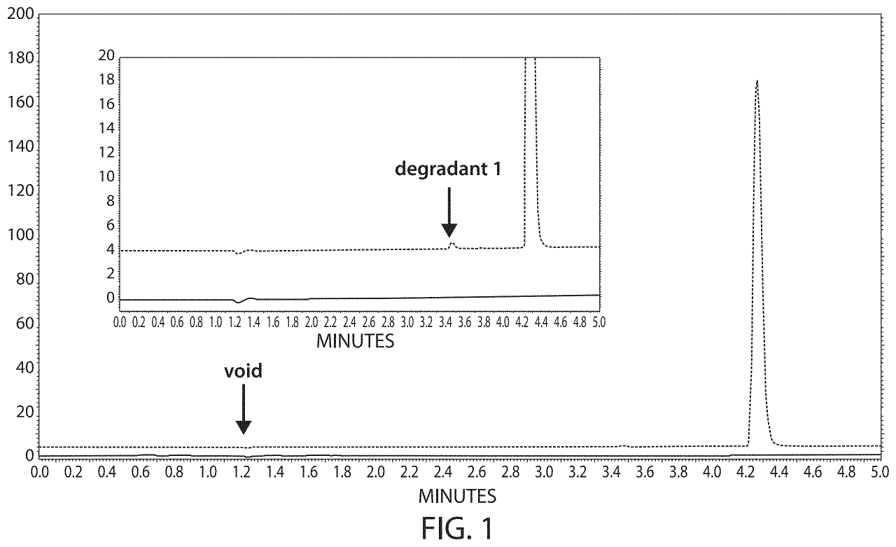

The HPLC method parameters are summarized in Table 1. A representative chromatogram of Compound 1 is shown in FIG. 1.

TABLE 1

| Summary of method parameters for analysis of Compound 1 | | | |
|---|---|---|---|
| Column | MAC-Mod Halo C18, | | |
| | 4.6 × 100 mm, 2.7 μm | | |
| Mobile Phase A | 99.9% water + 0.1% TFA | | |
| Mobile Phase B | 99.9% ACN + 0.1% TFA | | |
| Pump Gradient Program | Time (min) | % A | % B |
| | 0.0 | 80 | 20 |
| | 0.1 | 80 | 20 |
| | 5.1 | 40 | 60 |
| | 5.2 | 0 | 100 |
| | 6.2 | 0 | 100 |
| | 6.3 | 80 | 20 |
| | 10 | 80 | 20 |

| | |
|---|---|
| Diluent | water |
| Flow (mL/min) | 1 |
| Column Temperature | 35 |
| Injection Volume (μL) | 10 |
| Sample Temperature | Room temperature or 5° C. |
| | (application dependent) |
| Detection Wavelength | 280 |

### Example 3. Experimental Setup for Testing the Equilibrium Solubility of Compound 1 in Common Buffers

The equilibrium solubility of Compound 1 was tested in common buffers (Table 2) at a buffer strength of c(buffer)=200 mM. For this, samples of Compound 1 with c(Compound 1, target)=16 mg/mL were prepared in the different buffer solutions (Table 3), mixed by vortexing and inspected for solid residues. For the cases for which no solid residues were observed the solutions were arbitrarily supplemented with additional solid Compound 1. This process was repeated (hourly observation) until a solid residue remained readily observable. The samples were rotated at room temperature for 24 h.

TABLE 2
Buffer conditions and samples subjected to equilibrium solubility analysis

| | | | | | | Additional |
|---|---|---|---|---|---|---|
| | | | pH | c(Compound 1, | c(Compound 1, | Compound 1 |
| | | c(Buffer) | (Buffer, | FB, Target) | FB, Actual) | Solid |
| Sample # | Buffer System | [mM] | Actual) | [mg/mL] | [mg/mL] | Added |
| 100 | Phosphoric acid/Sodium | 200 | 2.00 | 16.0 | 16.4 | Yes |
| | phosphate (monobasic) | | | | | |
| 101 | Citric acid/Sodium | 200 | 3.00 | 16.0 | 16.4 | Yes |
| 102 | Citric acid/Sodium | 200 | 4.00 | 16.0 | 16.6 | No |
| 103 | Acetic acid/Sodium | 200 | 5.00 | 16.0 | 17.6 | No |
| | Acetate | | | | | |
| 104 | Sodium phosphate | 200 | 6.00 | 16.0 | 17.9 | No |
| | (monobasic/dibasic) | | | | | |
| 105 | Sodium phosphate | 200 | 7.00 | 16.0 | 16.7 | No |
| | (monobasic/dibasic) | | | | | |
| 106 | Sodium phosphate | 200 | 8.00 | 16.0 | 16.6 | No |
| | (monobasic/dibasic) | | | | | |

The equilibrium solubility samples were cleared by centrifugation (table top centrifuge, rcf=16,100×g, 10 min). The supernatant was subjected to HPLC analysis and assayed in triplicates for Compound 1 concentration using the HPLC method described in Example 2. The pH of the supernatant was measured (Table 3).

TABLE 3
Equilibrium solubility data in different buffer solutions

| | | | | | | | Main |
|---|---|---|---|---|---|---|---|
| | | | Avg. | | | | Degradant |
| | | Dil. | Compound 1 | Avg. | | | Peak Area |
| | | Factor | Retention | Compound 1 | Avg. | | as % of |
| pH | pH | (HPLC | Time | Peak Area | c(Compound 1, | StDev | Parent Peak |
| (Buffer) | (Meas.) | Samples) | [min] | [mAU²] | FB) [mg/mL] | c(Compound 1) | Area |
| 2.00 | 1.29 | 100 | 4.12 | 7552644 | 158.74 | 3.6E−03 | 1.48 |
| 3.00 | 2.72 | 100 | 4.21 | 302520 | 5.96 | 6.3E−04 | 3.46 |
| 4.00 | 3.74 | 100 | 4.22 | 313683 | 6.20 | 2.5E−04 | 3.10 |
| 5.00 | 4.57 | 100 | 4.20 | 374682 | 7.48 | 3.8E−04 | 4.21 |
| 7.00 | 6.33 | 100 | 4.20 | 588211 | 11.98 | 1.1E−03 | 3.01 |
| 8.00 | 6.67 | 100 | 4.20 | 746250 | 15.31 | 2.0E−03 | 6.55 |

### Example 4. Experimental Setup for Testing the pH1-Dependent Stability and pH1-Rate Profile of Compound 1

The stability Compound 1 was assessed over a 10-day period at several pH-values pH=2, 3, 4, 5, 6, 7, 8) and at two different temperatures (4° C., 40° C.). Samples of Compound 1 at c(Compound 1)=0.1 mg/mL were prepared in standard buffers (Table 2) at a buffer strength of c(buffer)=100 mM and incubated at either T=4° C. or T=40° C. (exclusion from light during incubation and analysis). The concentration of Compound 1 was assessed in such samples via HPLC analysis at an initial time point (to) and on days 1, 2, 3, 4, 7 and 10 (Δt=day×24 h). The pH was monitored at identical time points during the 10-day period.

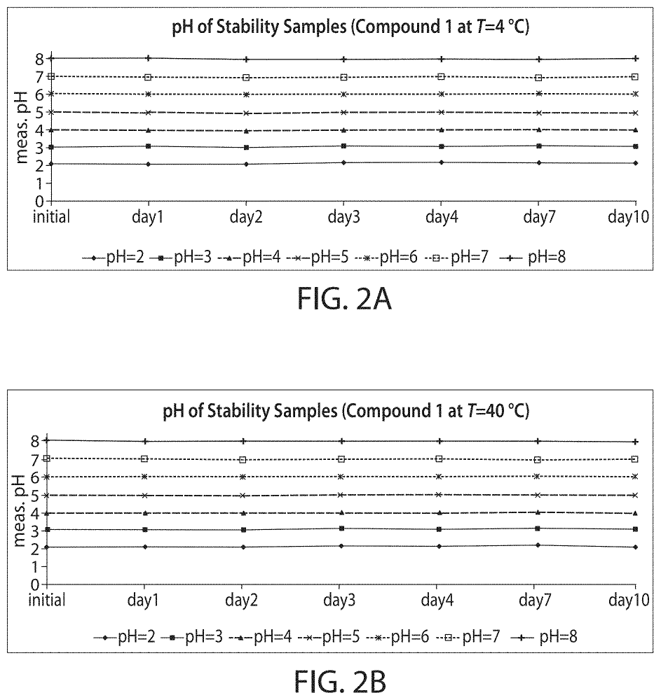

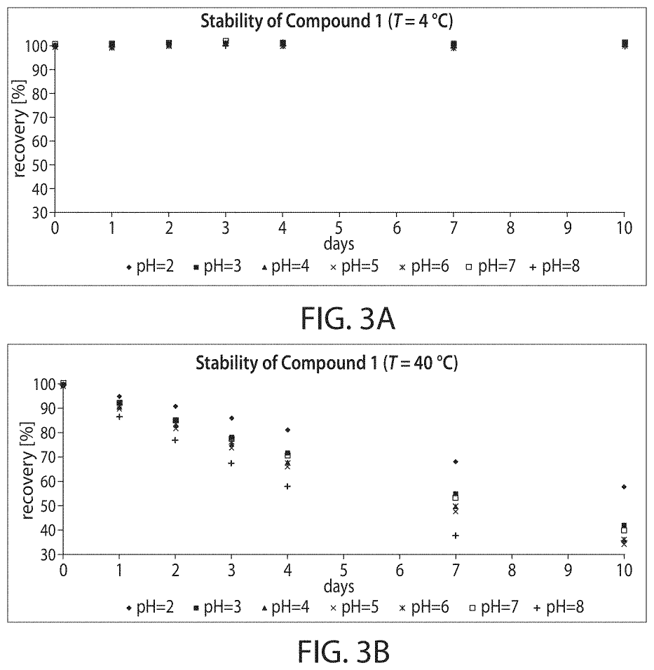

Over the 10-day period for both temperatures the pH of the sample solutions (FIG. 2A & FIG. 2B) was monitored and the recovery (R %) of Compound 1 (FIG. 3A & FIG. 3B) was calculated via:

$$R \% = ( \frac{c ( Compound 1 , theor . )}{c ( Compound 1 , actual )} ) \cdot 100 \qquad ( 1 )$$

whereas c(Compound 1, theor.)=0.1 mg/mL and c(Compound 1, actual) is determined by HPLC analysis via integration of the Compound 1 parent peak areas.

Compound 1 is stable in buffered aqueous solutions over abroad pH-range (pH=2-8) when stored in the dark at 4° C. At elevated temperature (T=40° C., in the dark) significant degradation is observed. The trend of the stability/pH-correlation is clear with Compound 1 tending to be more stable towards a low pH and less stable towards a higher pH. It can be excluded that the differences observed in stability can be attributed to changes in pH since the pH-values of the studies solutions remained constant within the 10 days of the experiment. All solutions were clear throughout the course of the experiment; no precipitation of the drug compound could be observed.

### Example 5. Solid State Characterization of Compound 1.HCl

An exemplary sample of the solid state of Compound 1.HCl was assessed by powder X-ray diffractometry (XPRD) using a Rigaku Miniflex X-ray diffractometer (Cu-Kα source, NaI-Scintillation Counter, U=30 kV, I=15 mA).

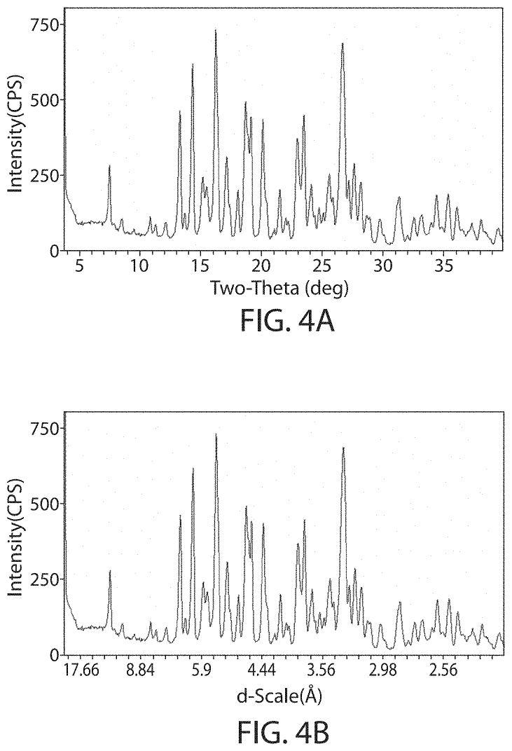

The solid Compound 1.HCl (m=15-20 mg) was analyzed in a zero-background holder silicon (Si). Scanning was carried out in FT-mode with a 2θ-scan (2θ=3°→40°, Δ2θ=0.05°, t_{count}=20 s). The obtained diffractogram is displayed in FIG. 4A on a 20-scale and as specimen dimensions in real-space (d-scale) as shown in FIG. 4B. Additionally, representative peaks from an exemplary XRD pattern of Compound 1.HCl can be indicated by their values of 2θ, d-spacing, and relative intensities, for example, in Table 4 below. At low d the indication of a plateau of total reflection indicated the proper sample alignment.

The defined peak pattern obtained for Compound 1.HCl shows the drug compound as crystalline, which makes likely that a crystalline drug product will result from solely pairing the drug compound with crystalline excipients like mannitol during a lyophilization process. The use of amorphous excipients on the other hand might shift a crystallization process towards an amorphous drug product potentially comprising enhanced properties regarding reconstitution, dissolution and solubility

TABLE 4
Selected experimental powder XRD pattern
data for Compound 1•HCl

| 2θ (degrees) | d-spacing (Å) | Relative Intensity (%) |
|---|---|---|
| 3.744 | 23.5791 | 100 |
| 7.539 | 11.7173 | 13.7 |
| 13.295 | 6.654 | 11.7 |
| 14.343 | 6.1703 | 19 |
| 16.256 | 5.4483 | 24.8 |
| 18.751 | 4.7284 | 21.2 |
| 19.151 | 4.6307 | 12.5 |
| 20.157 | 4.4018 | 17.2 |
| 23.539 | 3.7764 | 16.6 |
| 26.702 | 3.3358 | 19.1 |

### Example 6. Experimental Setup and Details for Testing of Vehicles

Two additional vehicles: sodium citrate (50 mM) and PBS (commercial) were formulated at different concentrations (Table 5, Table 6). Both vehicles at any concentration were compounded using the appropriate final sodium hydroxide concentration (0.1 M NaOH stock solution).

TABLE 5
Compounding information for citrate and PBS formulations

| | | | | Buffer | | | | Osml. |
|---|---|---|---|---|---|---|---|---|
| Sample | c(Compound 1, FB) | | V | Stock | c(HCl) | Target | final | [mOsm/ |
| # | [mg/mL] | Conditions | [mL] | Conc. | mM | pH | pH | kg] |
| 26 | 0.5 | PBS, after | 4 | 10× | — | 7.00 | 7.00 | 311 |
| | | compounding | | | | | | |
| 27 | 0.5 | PBS | 4 | 10× | 1.69 | 6.80 | 6.78 | 309 |
| | | | | Buffer | | | | Osml. |
| Sample | c(Compound 1, FB) | | V | Stock | c(NaOH) | Target | final | [mOsm/ |
| # | [mg/mL] | Conditions | [mL] | Conc. | mM | pH | pH | kg] |
| 28 | 1.0 | Citrate | 5 | 10× | 3.75 | 6.80 | 7.08 | 154 |
| 29 | 10.0 | Citrate | 2 | 10× | 37.5 | 6.80 | 6.82 | 216 |

TABLE 6
Concentration and recovery for citrate and PBS formulations (HPLC analysis)

| | | c(Compound 1, | Avg. Compound 1 | Avg. Compound 1 | Avg. c(Compound 1, | StDev. | Avg. % | |
|---|---|---|---|---|---|---|---|---|
| Sample | | FB, Theor.) | Retention Time | Peak Area | FB,Obs.) | c(Compound 1, | Compound 1 | pH |
| # | Conditions | [mg/mL] | [min] | [mAU²] | [mg/mL] | FB, Obs.) | Recovery | (Meas.) |
| 26 | After | 0.5 | 4.21 | 483261 | 0.5 | 1.6E−03 | 103 | 7.00 |
| | compounding | | | | | | | |
| 26 | After filtration | 0.5 | 4.21 | 482115 | 0.5 | 2.2E−03 | 103 | n/a |
| 27 | After | 0.5 | 4.21 | 487381 | 0.5 | 1.2E−03 | 104 | 6.78 |
| | compounding | | | | | | | |
| 27 | After filtration | 0.5 | 4.21 | 483822 | 0.5 | 2.2E−03 | 103 | n/a |
| 28 | After | 1.0 | 4.19 | 505228 | 1.1 | 0.00 | 106 | 7.08 |
| | compounding | | | | | | | |
| 28 | After filtration, | 1.0 | 4.19 | 502493 | 1.1 | 0.02 | 105 | n/a |
| | t0 | | | | | | | |
| 28 | t = 12 h | 1.0 | 4.19 | 506190 | 1.1 | 0.01 | 106 | 7.08 |
| 28 | t = 12 h, | 1.0 | 4.18 | 500765 | 1.1 | 0.00 | 105 | n/a |
| | +filtration | | | | | | | |
| 28 | t = 24 h | 1.0 | 4.18 | 485904 | 1.0 | 0.01 | 101 | 7.01 |
| 28 | t = 24 h, | 1.0 | 4.19 | 480614 | 1.0 | 0.00 | 100 | n/a |
| | +filtration | | | | | | | |
| 29 | After | 10.0 | 4.18 | 478936 | 10.0 | 0.03 | 100 | 6.82 |
| | compounding | | | | | | | |
| 29 | After filtration, | 10.0 | 4.19 | 467520 | 9.7 | 0.03 | 97 | n/a |
| 29 | t = 12 h | 10.0 | 4.18 | 472523 | 9.8 | 0.04 | 98 | 6.86 |
| 29 | t = 12 h, | 10.0 | 4.18 | 464674 | 9.6 | 0.02 | 96 | n/a |
| | +filtration | | | | | | | |
| 29 | t = 24 h | 10.0 | 4.18 | 484808 | 10.1 | 0.39 | 101 | 6.79 |
| 29 | t = 24 h, | 10.0 | 4.18 | 465309 | 9.7 | 0.01 | 97 | n/a |
| | +filtration | | | | | | | |

The PBS vehicle was compounded to two different pH-values while the citrate vehicle was compounded at different concentrations at the same target pH and tested for its stability over the course of 24 h (rt, exclusion from light). To evaluate the potential occurrence of particles from precipitation, a filtration step (0.2 μm micro centrifugal filters) was included at each time point.

In case of Sample #27 the amount of HCl(aq.) (0.1 M stock) added instead of the NaOH-solution was mixed with the 10× PBS buffer and added to the aqueous solution of Compound 1 in a single step.

In the case of PBS formulations at a low concentration of Compound 1 with c(Compound 1)=0.5 mg/mL, the concentrations of HCl(aq.) or NaOH(aq.) were carefully determined in iterative compounding titrations (see above). Stability of Compound 1 in citrate buffer was confirmed for □t=24 h; losses are comparable to the degradation generally observed for Compound 1 under the applied conditions (ambient temperature). Precipitation of Compound 1 was not observed within this time frame.

### Example 7. Development of the Lyophilization Process

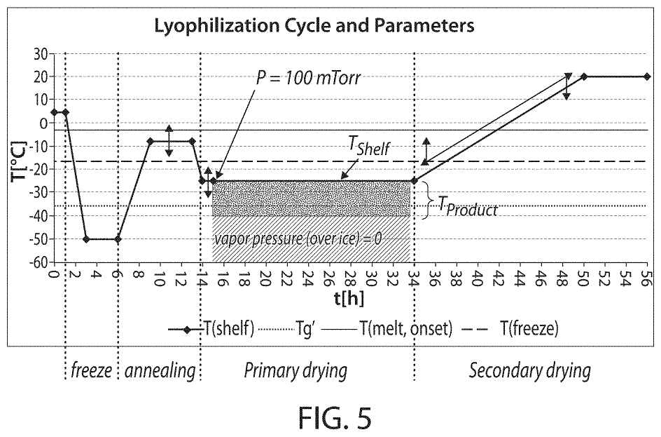

A conservative lyophilization cycle (FIG. 5) was developed for lyophilization of the Compound 1 target formulation. The lyophilization cycle comprises an annealing step and a primary drying time of 20 h. During cycle development the annealing temperature as well as the primary drying temperature (shelf temperature) were varied to achieve optimal drying properties and economic use of time. FIG. 5 shows the shelf temperature (T_{shelf}) of the lyophilizer as well as the exemplary parameters of an early stage formulation of Compound 1 with Tg′ as the glass transition temperature, T_{(melt, onset)} as the onset temperature of melting and T_{(freeze)} as the freezing temperature of the formulation as measured by differential scanning calorimetry (DSC).

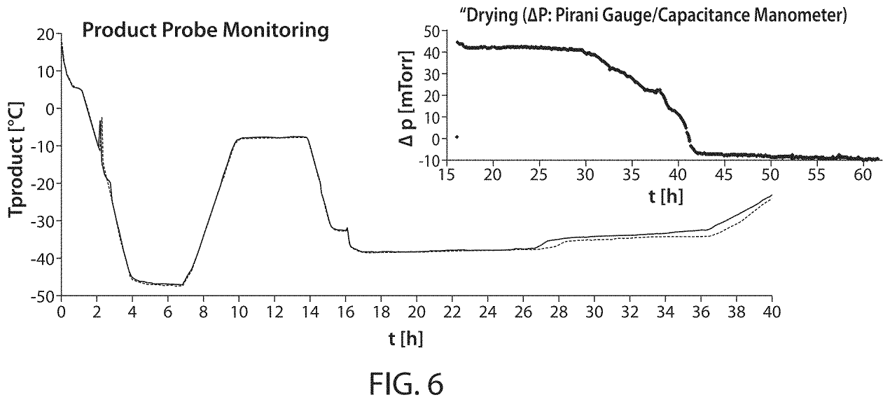

Due to the evaporation enthalpy of the aqueous phase during the drying process (100 mTorr) the product temperature T_{Product} is generally lower than the shelf temperature of the lyophilizer; below a temperature of T=−40° C. the water vapor over ice is close to zero. Variation of the shelf temperature was performed to achieve a slow primary drying with a primary drying temperature below the Tg′ (amorphous product) or the eutectic melting temperature (Tₑᵤ, crystalline product) of the formulation but above a T_{Product}>−40° C. During test lyophilizations the product vials were equipped with temperature sensors to record T_{Product}, in the final cycle a T_{Product}=−38.5° C. was observed at T_{Shelf}=−35° C. (FIG. 6). The end of primary drying was determined by measuring the water vapor release over time as a function of the pressure differences between the product chamber and the vacuum circuit of the lyophilizer (FIG. 6). It is noted that in the presence of certain excipients (e.g., mannitol) and at certain concentrations of those excipients primary drying can be accomplished well above a Tg′ or Tₑᵤ.

Parameters of the optimized lyophilization cycle can be found in Table 7. The residual moisture content of lyophilized formulation vehicles was determined by thermogravimetric analysis (TGA) with approximately 1.5-1.7% (w/w).

TABLE 7
Details of the lyophilization cycle program

| | | | | | | Step | |
|---|---|---|---|---|---|---|---|
| | | | Set Point, | Ramp Rate | Pressure | Time | |
| Step # | Phase | Type | (T_{shelf)} [° C.] | [° C./h] | [mTorr] | [min] | Total Time [h] |
| 0 | Loading | — | 5 | — | atm | — | — |
| 1 | Freeze | Hold | 5 | 0.0 | atm | 60 | 1.00 |
| 2 | Freeze | Rate | −50 | 20.0 | atm | 165 | 3.75 |
| 3 | Freeze | Hold | −50 | — | atm | 180 | 6.75 |
| 4 | Freeze/Anneal | Rate | −8 | 20.0 | atm | 174 | 9.65 |
| 5 | Freeze/Anneal | Hold | −8 | — | atm | 240 | 13.65 |
| 6 | Freeze/Anneal | Rate | −35 | 20.0 | atm | 81 | 15.00 |
| 7 | Freeze/Anneal | Hold | −35 | — | atm | 60 | 16.00 |
| 8 | Primary Drying | Hold | −35 | — | 100 | 1200 | 36.00 |
| 10 | Secondary drying | Rate | 20 | 2.5 | 100 | 1300 | 57.67 |
| 11 | Secondary drying | Hold | 20 | — | 100 | 360 | 63.67 |

The developed lyophilization cycle was successfully applied to Compound 1 formulations.

### Example 8. Exemplary Formulations

The target product profile (TPP) was defined as:
c(Compound 1)=10 mg/mL
Sodium phosphate buffer, pH=6.8, =270-320 mOsm/kg
Minimal number and minimal concentration of excipients (bulking agents, solubilizing agents) that facilitate robust preparation of a lyophilized drug product
Neutralization of the Compound 1.HCl during liquid compounding during preparation of (the lyophilization fill solutions

A limited formulation matrix of 108 formulations was created. The matrix comprised bulking agents, co-solvents, cyclodextrins at varying concentrations (Table 8); the concentration of sodium phosphate (10 mm) and the API (10 mg/mL) were kept constant.

TABLE 8
Excipients evaluated in the limited formulation testing matrix

| | | Approx. Conc. |
|---|---|---|
| Excipient | Category | [%], w/w |
| Mannitol | bulking agent | 4.0. 3.5. 3.0. 2.5 |
| Lactose | bulking agent | 8.0, 7.0, 6.0, |
| | | 5.0, 1.5 |
| HPβCD | bulking/solubilizing | 5.0, 2.1, 0.7, 0.0 |
| | (encapsulating) agent | |
| Captisol | bulking/solubilizing | 5.6, 2.4, 0.8, 0.0 |
| (Sulfobutyl ether | (encapsulating) agent | |
| β-cyclodextrin) | | |
| PEG 400 | solubilizing agent | 0.05, 0.00 |
| | (cosolvent) | |
| TWEEN 20 | solubilizing agent | 0.01, 0.00 |
| | (cosolvent) | |

Formulations always contained mannitol or lactose as a bulking agent and varying amounts of cyclodextrins and cosolvents. Usually the concentration of the bulking agent was varied to maintain an osmolality in the appropriate range. Lyophilization at an increased total volume concordant with a lower concentration of all formulation components compared to the final reconstitution strength was evaluated.

The 108 formulations were compounded from stock solutions including neutralization of Compound 1.HCl and lyophilized in triplicates and duplicates on a 1 mL scale using 5 mL lyophilization vials. The concentration of excipients denoted in percent w/w is relative to the weight of Compound 1. The concentration of excipients denoted in percent (w/w or w/v) can only be considered to be approximate and do not reflect absolute w/w or w/v percentages, since neutralization compounding from stock solutions involves dilutions that do not account for defined mass or volume-ratios.

The following formulations represent the top, equally well-performing candidates with the mannitol containing formulation providing a more elegant cake structure and the lactose formulation achieving a cleaner, less foamy reconstitution.
Formulation 1: 10 mg/mL Compound 1, 10 mM sodium phosphate, pH=6.8, 5% HPβCD, 3% Mannitol
Formulation 2: 10 mg/mL Compound 1, 10 mM sodium phosphate, pH=6.8, 5% HPβCD, 3% Mannitol, 0.05% PEG400
Formulation 3: 10 mg/mL Compound 1, 10 mM sodium phosphate, pH=6.8, 5% HPβCD, 5% Lactose
Formulation 4: 10 mg/mL Compound 1, 10 mM sodium phosphate, pH=6.8, 5% HPβCD, 5% Lactose, 0.05% PEG400
Formulation 5: 10 mg/mL Compound 1, 10 mM sodium phosphate, pH=6.8, 3.5% HPβCD, 3% Mannitol
Formulation 6: 10 mg/mL Compound 1, 10 mM sodium phosphate, pH=6.8, 3.5% HPβCD, 5% Lactose

### Example 9. Preparation of Formulation 5 at an Increased Bench Scale

Formulation 5 was prepared to the target concentration of c(Compound 1)=10 mg/mL on a 210 mL scale. First, a liquid fill solution was compounded including neutralization of the Compound 1.HCl component before lyophilization containers were filled and lyophilization was performed. Amber lyophilization containers at V_{container}=20 mL and a fill volume of V_{fill}=5 mL were applied.

The fill solution was compounded from the following stock solutions at the given multifold concentration:

i. Aq. Compound 1 stock solution, c(Compound 1)=40 mg/mL, [4×]
ii. 10% aq. mannitol stock solution (w/v), [3.33×]
iii. 28% aq. HPβCD stock solution (w/w), [8×]
iv. 500 mM aq. sodium hydroxide stock solution [12.82×]
v. 100 mM aq. sodium phosphate buffer, pH=6.8 [10×].

For preparation of the fill solution the residual volume of water to reach the target concentrations was added to the Compound 1 stock solution under constant stirring. Subsequently the mannitol and HPβCD stock solutions were dispensed into the mixture before the sodium hydroxide stock solution was added to yield a final c(NaOH)=39 mM. In the last step the 10× sodium phosphate buffer was added and the solution was allowed to cool to ambient temperature.

The mannitol, HPβCD and buffer stock solutions were filtered (0.2 μm PES membrane, 20 mm syringe filter, Acrodisc Supor EKV) prior to compounding without observing any difficulties. The ready-compounded lyophilization fill solution was likewise filtered (0.2 μm PES membrane, 20 mm syringe filter, Acrodisc Supor EKV) before dispensing into lyophilization vials under best clean conditions.

The 40 resulting lyophilization vials were arranged densely packed at the center of the lyophilization and placed at the center shelf of the lyophilizer product chamber. Product vials were surrounded by vials filled with buffer solution. The developed lyophilization cycle from Example 7 was applied for lyophilization of the product vials; vials were stoppered manually after vacuum release to ambient air.

### Example 10. Reconstitution and Reconstitution Stability of the Lyophilized Compound 1 Drug Product

The lyophilized Compound 1 drug product was subjected to testing of reconstitution and to stability testing for 6 h after reconstituted with DI water V_{DIwater}=5 mL.

The lyophilization cake readily reconstituted within 10-20 s. The solution during reconstitution appeared quite foamy and contained many bubbles, which cleared within approximately 2 min addition of the reconstitution solution. Residual micro-bubbles on the container wall can be removed by vortexing (2 s) or sonication (2 s). The reconstituted solution appears clear and colorless. The pH of the reconstituted solution was measured with pH=6.81; the osmolality was determined to φ=295 mOsm/kg. The reconstituted solution was practically free of particulates as determined by liquid particle counting (LPC, HIAC: Vₛₐₘₚₗₑ=5 mL, nᵣᵤₙₛ=4, 1^{st} run discarded, f_{dilution}=10, V_{nominal, container}=2 mL) with a cumulative count of 3200 particles at a size of 10 μm and 667 particles at a size of 25 μm.

The recovery of Compound 1 was monitored for 24 h after reconstitution to test for the in-use stability of the reconstituted solution (Table 9)

TABLE 9
Excipients evaluated in the limited formulation testing matrix

| | | Avg. | Avg. | Avg. | | | |
|---|---|---|---|---|---|---|---|
| | c(Compound 1, | Compound 1 | Compound 1 | c(Compound 1, | StDev. | Avg. % | |
| | FB, Theor.) | Retention Time | Peak Area | FB, Obs.) | c(Compound 1, | Compound 1 | pH |
| Conditions | [mg/mL] | [min] | [mAU²] | [mg/mL] | FB, Obs.) | Recovery | (Meas.) |
| Reconst, t₀ | 9.65* | 4.22 | 464901 | 9.65 | 0.78 | 100* | 6.80 |
| Reconst, | 9.65 | 4.22 | 456448 | 9.47 | 0.12 | 98 | 6.79 |
| t = l h | | | | | | | |
| Reconst, | 9.65 | 4.22 | 444244 | 9.20 | 0.09 | 95 | 6.80 |
| t = 2 h | | | | | | | |
| Reconst, | 9.65 | 4.21 | 850110^{#} | 9.19 | 0.28 | 95 | 6.79 |
| t = 4 h | | | | | | | |
| Reconst, | 9.65 | 4.22 | 448263 | 9.28 | 0.06 | 96 | 6.79 |
| t = 6 h | | | | | | | |
| Reconst, | 9.65 | 4.21 | 434893 | 8.99 | 0.01 | 93 | 6.85 |
| t = 24 h | | | | | | | |

*% recovery (Compound 1) [symbol] 100%, since V_{fill} solution = V_{DIwater, reconst.}; ^{#}different dilution was applied for preparation of the HPLC sample

The current phosphate-based formulation containing HPβCD and mannitol shows a loss in Compound 1 recovery of approximately 7% over the course of 24 h.

### Example 11. Compatibility with Infusion Vehicles and in Use Storage of Reconstituted Compound 1 Formulation

The compatibility of the reconstituted Compound 1 formulation with two infusion vehicles, normal saline (NS) and 5% dextrose in water (D5W) was tested after Δt=4 h for two concentration (high/low) at c(Compound 1, target)=0.1 mg/mL and c(Compound 1, target)=1.0 mg/mL and at ambient conditions (Table 10). The average values of % recovery are relative to the dilution of the reconstituted drug product into DI water at t₀.

TABLE 10
Assessment of compatibility with infusion vehicles

| | | Avg. | | | | |
|---|---|---|---|---|---|---|
| | | Compound 1 | Avg. | Avg. | | |
| | c(Compound 1, | Retention | Compound 1 | c(Compound | StDev. | Avg. % |
| | FB, Theor.) | Time | Peak Area | 1, FB, Obs.) | c(Compound | Compound 1 |
| Conditions | [mg/mL] | [min] | [mAU²] | [mg/mL] | 1, FB, Obs.) | Recovery |
| Water, t₀ | 0.09* | 4.22 | 449346 | 0.09 | 6 · 10⁻⁰⁵ | 100 |
| Water, t = 4 h | 0.09 | 4.22 | 448417 | 0.09 | 4 · 10⁻⁰⁵ | 100 |
| NS, t = 4 h | 0.09 | 4.21 | 450839 | 0.09 | 5 · 10⁻⁰⁵ | 100 |
| D5W, t = 4 h | 0.09 | 4.21 | 445119 | 0.09 | 2 · 10⁻⁰⁴ | 100 |
| Water, t₀ | 0.92* | 4.22 | 445348 | 0.92 | 3 · 10⁻⁰³ | 100 |
| Water, t = 4 h | 0.92 | 4.22 | 444601 | 0.92 | 6 · 10⁻⁰⁴ | 100 |
| NS, t = 4 h | 0.92 | 4.21 | 446396 | 0.92 | 3 · 10⁻⁰³ | 100 |
| D5W, t = 4 h | 0.92 | 4.21 | 446458 | 0.92 | 1 · 1⁻⁰³ | 99 |

*% recovery (Compound 1) [symbol] 100%, since V_{fill solution} = V_{DIwater, reconst.};

Both infusion, NS, and D5W are compatible with the reconstituted Compound 1 drug product at the tested concentrations and within the course of 4 h at ambient temperature and lighting conditions.

### Example 12. Compatibility of Reconstituted Compound 1 Formulation with Vials and Stoppers

The compatibility of the reconstituted Compound 1 formulation with sterile manufacturing vials and stoppers (Table 17) was tested for a contact time of Δt=1 h at c(Compound 1, target)=10 mg/mL at ambient conditions. The average values of % recovery are relative to the reconstituted drug product that has not been in contact with the tested materials.

TABLE 11
Assessment of compatibility with vials and stoppers for manufacturing

| | | Avg. | | | | |
|---|---|---|---|---|---|---|
| | | Compound 1 | Avg. | Avg. | | |
| | c(Compound 1, | Retention | Compound 1 | c(Compound | StDev. | Avg. % |
| | FB, Theor.) | Time | Peak Area | 1, FB, Obs.) | c(Compound | Compound 1 |
| Conditions | [mg/mL] | [min] | [mAU²] | [mg/mL] | 1, FB, Obs.) | Recovery |
| Reconst. Soln., | 9.32* | 4.21 | 450793 | 9.32 | 0.02 | 100* |
| Vial, t = l h | 9.32 | 4.21 | 449081 | 9.28 | 0.02 | 100 |
| Stopper, t = l h | 9.32 | 4.20 | 449072 | 9.28 | 0.01 | 100 |

*% recovery (Compound 1) [symbol] 100%, since V_{fill solution} = V_{DIwater, reconst}.

Table 11 shows the tested vial and stopper material is compatible with the reconstituted Compound 1 drug product at the tested concentrations and within the course of 1 h at ambient temperature and lighting conditions.

### Example 13. Compatibility of Reconstituted Compound 1 Formulation with Sterilization Filters

The feasibility of aseptic processing was evaluated and three different filter materials were tested for compatibility with the reconstituted Compound 1 formulation. Aseptic processing is the generally suggested method for sterilization of Compound 1, since Compound 1 comprises a decreased stability at elevated temperature and only dry sterilization cycles (T=160° C., t=120 min) are applicable for lyophilized drug product as a terminal sterilization option.

Filter compatibility of the reconstituted Compound 1 drug product was tested with different filter material (Table 17) composed of polyethersulfone (PES), nylon, and polyvinylidene fluoride (PVDF). For this a volume of V_{filter pass}=10 mL was passed through the respective filter and the first as well as the last 10 vol % of the filtered volume were assayed for recovery of Compound 1 (Table 12).

TABLE 12
Assessment of compatibility with sterilization filters

| | c(Com- | Avg. | Avg. | Avg. | |
|---|---|---|---|---|---|
| | pound | Com- | Com- | c(Com- | Avg. |
| | 1, | pound 1 | pound 1 | pound 1, | % Com- |
| | FB, | Retention | Peak | FB, | pound |
| | Theor.) | Time | Area | Obs.) | 1 |
| Conditions | [mg/mL] | [min] | [mAU²] | [mg/mL] | Recovery |
| Sample Soln. | 0.95* | 4.14 | 4296856 | 0.95 | 100* |
| (pre-filtration) | | | | | |
| PES, first 10% | 0.95 | 4.15 | 4285782 | 0.94 | 100 |
| PES, last 10% | 0.95 | 4.14 | 4296190 | 0.95 | 100 |
| Nylon, first 10% | 0.95 | 4.15 | 4255708 | 0.94 | 99 |
| Nylon, last 10% | 0.95 | 4.14 | 4297770 | 0.95 | 100 |
| PVDF, first 10% | 0.95 | 4.13 | 4306693 | 0.95 | 100 |
| PVDF, last 10% | 0.95 | 4.13 | 4305766 | 0.95 | 100 |

*%recovery(Compound 1) [symbol] 100%, since V_{fill solution} = V_{DIwater, reconst.}

The average values of % recovery are relative to the reconstituted drug product that has not been in contact with the tested materials. No increase in back-pressure was observed with any of the tested filters. At the studied filter pass volumes and concentrations no significant loss in Compound 1 recovery was observed.

### Example 14. Compatibility of Reconstituted Compound 1 Formulation with Infusion Bags and IV Systems

The compatibility of the reconstituted Compound 1 formulation with two infusion bags (different volumes and material) and two IV lines was tested (Table 13) at c(Compound 1)=0.1 mg/mL. After injection of the reconstituted drug product solution into infusion bags filled with NS, the recovery of Compound 1 was measured i) directly after mixing with the infusion vehicle (to, Table 14) and ii) after Δt=10 min of contact time with the infusion bag (Table 15). Additionally, storage in an infusion bag at ambient temperature and lighting conditions was assessed for Δt=6 h (Table 15). The average values of % recovery are relative to the reconstituted drug product that has not been in contact with the tested materials.

TABLE 13
Tested infusion bag and IV system material

| | Size | | | Article #/ | |
|---|---|---|---|---|---|
| Material | [mL] | Manufact. | Description | Catalog # | Lot # |
| Inf. | 500 | Baxter | 0.9% Sodium | FE1323 | 14J15E3D |
| bag #1 | | | Chloride | | |
| | | | Injection USP | | |
| | | | (500 mL | | |
| | | | Viaflo Bag) | | |
| Inf. | 50 | Baxter | 0.9% Sodium | 2B1301 | P326306 |
| bag #2 | | | Chloride | | |
| | | | Injection | | |
| | | | USP (50 mL | | |
| | | | Viaflex Bag) | | |
| IV | — | Baxter | Clearlink/ | EMS3110 | R14H27059 |
| system | | | Interlink, Non- | | |
| #1 | | | DEHP solution | | |
| | | | set, Duo-vent | | |
| | | | spike | | |
| IV | — | Baxter | Clearlink system, | 2C8519S | R14J10060 |
| system | | | continuo- | | |
| #2 | | | Flo solution set | | |

TABLE 14
Assessment of compatibility with infusion bags at t0.

| | | Avg. | | | | |
|---|---|---|---|---|---|---|
| | | Compound 1 | Avg. | Avg. | | |
| | c(Compound 1, | Retention | Compound 1 | c(Compound | StDev. | Avg. % |
| | FB, Theor.) | Time | Peak Area | 1, FB, Obs.) | c(Compound | Compound 1 |
| Conditions | [mg/mL] | [min] | [mAU²] | [mg/mL] | 1, FB, Obs.) | Recovery |
| Sample soln. | 0.09* | 4.19 | 441971 | 0.09 | 1.3E−01 | 100* |
| (water) | | | | | | |
| infus. bag1, t₀ | 0.09 | 4.19 | 423961 | 0.09 | 1.8E−04 | 96 |
| infus. bag2, t₀ | 0.09 | 4.19 | 393294 | 0.08 | 7.1E−05 | 88 |

*% recovery (Compound 1) [symbol] 100%, since V_{fill solution} = V_{DIwater, reconst.}

TABLE 15
Assessment of compatibility with infusion bags at t = 10 min and 6 h storage in infusion bag

| | | Avg. | | | | |
|---|---|---|---|---|---|---|
| | | Compound 1 | Avg. | Avg. | | |
| | c(Compound 1, | Retention | Compound 1 | c(Compound | StDev. | Avg. % |
| | FB, Theor.) | Time | Peak Area | 1, FB, Obs.) | c(Compound | Compound 1 |
| Conditions | [mg/mL] | [min] | [mAU²] | [mg/mL] | 1, FB, Obs.) | Recovery |
| infus. bag1, t₀ | 0.09* | 4.18 | 436470 | 0.09 | 2.7E−04 | 100* |
| infus. bag1, | 0.09 | 4.18 | 436694 | 0.09 | 3.6E−04 | 100 |
| t = 10 min | | | | | | |
| infus. bag1, | 0.09 | 4.19 | 436411 | 0.09 | 2.0E−04 | 100 |
| t = 6 h | | | | | | |
| infus. bag2, t₀ | 0.08* | 4.18 | 401640 | 0.08 | 1.1E−04 | 100* |
| infus. bag2, | 0.08 | 4.18 | 401638 | 0.08 | 5.2E−05 | 100 |
| t = 10 min | | | | | | |
| infus. bag2, | 0.08 | 4.19 | 400252 | 0.08 | 4.2E−04 | 100 |
| t = 6 h | | | | | | |

*% recovery (Compound 1) [symbol] 100%, since V_{fill solution} = V_{DIwater, reconst.}

The two IV systems were evaluated for compatibility with the reconstituted drug product solution by filling the IV lines with the diluted (NS) drug product solution and flowing V_{flow trough (FT)}=101 mL through the respective IV system at a flow rate of 5 mL/min. Flow-through samples of Vₛₐₘₚₗₑ=1 mL were collected i) immediately (V_{FT}=0 mL), ii) after V_{FT}=10 mL and iii) after V_{FT}=100 mL. Flow through samples were assayed for recovery of Compound 1 and compared to the infusion solution in the reservoir at t₀ (Table 16).

TABLE 16
Assessment of compatibility with IV systems

| | | Avg. | | | | |
|---|---|---|---|---|---|---|
| | | Compound 1 | Avg. | Avg. | | |
| | c(Compound 1, | Retention | Compound 1 | c(Compound | StDev. | Avg. % |
| | FB, Theor.) | Time | Peak Area | 1, FB, Obs.) | c(Compound | Compound 1 |
| Conditions | [mg/mL] | [min] | [mAU²] | [mg/mL] | 1, FB, Obs.) | Recovery |
| infus. bag, t₀ | 0.09* | 4.18 | 436470 | 0.09 | 2.7E−04 | 100* |
| (IV#1) | | | | | | |
| IV#1, VFT = | 0.09 | 4.18 | 436836 | 0.09 | — | 100 |
| 0 mL | | | | | | |
| IV#1, VFT = | 0.09 | 4.18 | 438353 | 0.09 | — | 100 |
| 10 mL | | | | | | |
| IV#1, VFT = | 0.09 | 4.18 | 440111 | 0.09 | — | 100 |
| 100 mL | | | | | | |
| infus. bag, t0 | 0.09* | 4.18 | 436640 | 0.09 | 1.1E−04 | 100* |
| (IV#2) | | | | | | |
| IV#2, VFT = | 0.09 | 4.18 | 436142 | 0.09 | — | 100 |
| 0 mL | | | | | | |
| IV#2, VFT = | 0.09 | 4.18 | 436115 | 0.09 | — | 100 |
| 10 mL | | | | | | |
| IV#2, VFT = | 0.09 | 4.19 | 436142 | 0.09 | — | 100 |
| 100 mL | | | | | | |

*% recovery (Compound 1) [symbol] 100%.

The tested infusion bags and IV system material is compatible with the reconstituted Compound 1 drug product at the tested concentrations and exposure times. While storage in the infusion bags up to 6 h does not change the concentration of Compound 1 in the respective infusion vehicle compared to t₀ (approximately 1 min after exposure), it seems to be the case that some Compound 1 material is adsorbed from the infusion bag material immediately after contact. Thereby, the observed changes in Compound 1 recovery do not correlate with the surface area of the tested infusion bags but seem to be dependent on the infusion bag material. Infusion bag #1 (Viaflo) shows a lesser extent of Compound 1 adsorption than infusion bag #2 (Viaflex).

Both IV systems appear to be inert towards binding of Compound 1; no changes in Compound 1 recovery were observed after flowing the Compound 1 solution derived from dilution of the reconstituted drug product solution through either IV test system.

### Example 15. Materials and Equipment

TABLE 17
Materials

| Material | Supplier/Manufactur | Part Number | Lot #/Serial # |
|---|---|---|---|
| 0.9% Sodium Chloride Injection | Baxter | 2B1301 | P326306 |
| USP (50 mL Viaflex Bag) | | | |
| 0.9% Sodium Chloride Injection | Baxter | FE1323 | 14J15E3D |
| USP (500 mL Viaflo Bag) | | | |
| Acetic Acid (glacial) | BDH | UN2789 | 2013042245 |
| Acetonitrile | Fisher Scientific | A998-4 | 146154/151811 |
| | | | / |
| Cannulas (Precision Glide | Becton Dickinson | 305175 | 6145499 |
| Needle D-D20G1)) | | | |
| Captisol (beta cyclodextrin | CyDex Inc. | — | NC-04A-05023 |
| sulfobutyl ethers, sodium salts) | | | |
| Citric acid anhydrous | Spectrum | Cl131 | XC0269 |
| Clearlink system, continuo-Flo | Baxter | 2C8519S | R14J10060 |
| solution set | | | |
| Clearlink/Interlink, Non-DEHP | Baxter | EMS3110 | R14H27059 |
| solution set, Duo-vent spike | | | |
| Crimp Seals (Afton Ready-To- | Afton Scientific | 54202258 | 1745-15 |
| Fill Sterilized Seals) | | | |
| Deionized water (ultra pure) | In-house, | Nanopure Infinity | (Barnstead) water |
| DSC pans (Pan + Lid, Hermetic | TA Instruments | T131003/T140603 | 900796.901/ |
| Alodined) | | | 900790.901 |
| Ethanol (dehydrated, 200 proof, | Spectrum | ET107 | ZT0426 |
| Undenatured, USP) | | | |
| Halo C18 HPLC column | MAC-MOD | 92814-602 | AH142237/ |
| | | | USRG003098 |
| HPLC (vials clear) | VWR | 46610-722 | 24108033 |
| HPLC vials (amber) | VWR | 46610-726 | 24107863 |
| Hydrochloric Acid | Fisher Scientific | A144-500 | 48038830 |
| Hydrogen Peroxide 35%, w/w, | Alfa Aesar | L14000 | W03A029 |
| stab. | | | |
| Hydroxypropyl-β-cyclodextrin | Spectrum | H2690 | 1D60239 |
| Lactose Monohydrate | Spectrum | LA106 | RO0212 |
| Lyophilization bottles | Wheaton | 223762 | 1550400 |
| (Wheaton, 20 mL, Serum | | | |
| bottles, Borosilicate glass, | | | |
| amber) | | | |
| Lyophilization stoppers | Wheaton | W224100-202 | 1539836 |
| (Wheaton, 20 mm, STPR, 3- | | | |
| Leg, Lyo, 13 × 20, GRY, BTYL, | | | |
| SLZD) | | | |
| Lyophilization vials (Wheaton, | Wheaton | 223685 | — |
| 5 mL vials, serum, Type I | | | |
| Borosilicate glass, clear) | | | |
| Lyophilization vials (Wheaton, | Wheaton | 223695 | 1548721 |
| 5 mL vials, serum, Type I | | | |
| Borosilicate glass, amber) | | | |
| Magnetic Stir Bars (various | VWR | — | — |
| sizes) | | | |
| Maleic acid | Spectrum | M1075 | VG1288 |
| Mannitol (USP) | Spectrum | MA165 | XE1369 |
| Manufacturing stoppers, sterile | West Pharmaceutical | 19700311 | D000039730 |
| (20 mm Lyo NovaPure RP | Services, Inc. | | |
| V10-F597W 4432/50 West | | | |
| ready pack) | | | |
| Manufacturing vials, sterile | Afton Scientific (via | 68000369 | 1775-15 |
| (Ready-to-fill Sterilized Vials, | West Pharmaceutical | | |
| 5 ML 20 MM S/L FNT W/BB | Services, Inc.) | | |
| PF WOS RU/RP | | | |
| Osmometry Std. 100 mOsm/kg | Fisher Scientific | 12-827-12 | 606261 |
| (Precision Systems No. 2101) | | | |
| Osmometry Std. 300 mOsm/kg | Fisher Scientific | 12-827-13 | 608182 |
| (Precision Systems No. 2103) | | | |
| Osmometry Std. 500 mOsm/kg | Fisher Scientific | 12-827-14 | 606262 |
| (Precision Systems No. 2105) | | | |
| pH Standard (pH 10.000) | Ricca Chemical | 1602-16 | 250 1A26 |
| | Company | | |
| pH Standard (pH 4.000) | Ricca Chemical | 1502-16 | 2410B96 |
| | Company | | |
| pH Standard (pH 7.000) | Ricca Chemical | 1552-16 | 2410761 |
| | Company | | |
| Phosphoric acid | Sigma Aldrich | 438081-500 ML | MKBL4262V |
| Polyethylene glycol 400, NF | Spectrum | PO110 | ZQ0106 |
| (PEG 400) | | | |
| Polysorbate 80, NF | Spectrum | PO1138 | 2DJ0314 |
| Prominence Autosampler Vial | Shimadzu | 228-45454-91 | 5454543230 |
| Closures | | | |
| Sodium acetate anhydrous | EMD | 7510 | 1775B65 |
| Sodium citrate•2H₂O | Acros Organics | 446330010 | A0342566 |
| Sodium hydroxide solution | VWR | BDH7247-1 | 410417 |
| 10N | | | |
| Sodium hydroxide solution | Fisher Scientific | SS276-1 | 100160 |
| N/10 | | | |
| Sodium phosphate dibasic | Spectrum | SO138 | WG0695 |
| anhydrous | | | |
| Sodium phosphate dibasic | Spectrum | S1108 | VM0472 |
| dihydrate | | | |
| Sodium phosphate monobasic | Spectrum | SO187 | VK1125 |
| anhydrous | | | |
| Sucrose (NF) | Spectrum | SU103 | RB0137 |
| Syringe filters (0.2 μm, 25 mm, | Pall | 4436 | 11961095 |
| Acrodisc, Nylon) | | | |
| Syringe filters (0.2 μm, 25 mm, | Pall | 4405 | 12491302 |
| Acrodisc, Supor EKV) | | | |
| Syringe filters (0.22 μm, 25 | Millipore | SLGVM33RS | R3SA41564 |
| mm, Milles GV, PVDF) | | | |
| Syringes Luer-Lock TIP (60 ml, | BD | 309653/-04/-46 | 8107397/ |
| 10 mL, 5 mL) | | | 5026529/ |
| TGA Pans (ceramic) | TA Instruments | T060516 | 952323.902 |
| Trifluoroacetic acid | EMD | TX1276-6 | 48038830 |
| TWEEN 20 | Fisher Scientific | BP337-500 | 117274 |
| Weighing pans | Fisher Scientific | NC0184742 | n/a |
| (Aluminum,12 mm) | | | |

TABLE 18
Equipment

| Equipment | Description | |
|---|---|---|
| Analytical Balance | Mettler Toledo MX-5, SN #1126472420 | |
| Bath-type Sonicator | Fisher Scientific, FS30 (100 W, 42 kHz), | |
| | S/N #RTA12119995B | |
| Biological Safety | NuAire Class II, Type A/B3, | |
| Cabinet | Model: Nu-425-600, S/N | |
| | #12757041800 | |
| Crimper | Wheaton, 20 mm, 224323 | |
| Decrimper | Wheaton, 20 mm, W225353 | |
| Differential | TA Instrument, DSC Q100 | |
| Scanning | | |
| Calorimeter | | |
| Digital Timer | VWR Laboratory Timer, Product | |
| | #62344-641, S/N #101445261 | |
| Environmental | ThermoForma 3911, S/N #40759-101 | |
| Chambers | ThermoForma 3911, S/N #57484-132 | |
| HIAC | Pacific Scientific, HIAC Royco | |
| | Model 9703 Liquid Particle | |
| | Counting System, Sensor: | |
| | HRLD400CE (serial#F06211); Pharm | |
| | Spec Software | |
| LC-MS/MS | Controller Unit: | Shimadzu Prominence CBM-20A |
| System | Pump System: | Shimadzu Prominence LC-20 AD |
| | Autosampler: | CTC HTS PAL |
| | Degasser: | Shimadzu Prominence DGU-20As |
| | MS Detector: | Applied Biosystems API 4000 |
| | Column Oven: | Shimadzu Prominence CTO-20A |
| | Software: | Applied Biosystems/MSD SCIEX |
| | Instruments: | Analyst Software |
| Lyophilizer | VirTis 25L Genesis SQ Super | |
| | XL lyophilizer, S/N #214950; | |
| | Encore iFIX Software V. 3.5 | |
| Magnetic Stir Plate | Barnstead/Thermolyne, Cimarec 2 (S46725) | |
| Micro Osmometer | Precision Systems, μOsmette, Mode1: 5004 | |
| Micro-pipettes | Rainin Instrument Co., Pipetmen P-10, P-20, | |
| | P-100, P-200, P-1000 and P-5000 | |
| pH-Meter/pH- | Thermo Scientific Orion Star A211, S/N #127 | |
| Electrode | VWR Symphony semi-micro | |
| | pH-electrode (epoxy) BNC, #89231- | |
| | 576 Thermo Scientific, Part #9810BN, | |
| | Lot # SS1 | |
| Photostability | Caron Photostability Chamber 6540-1, | |
| Chamber | S/N #011607-6540-1-8 | |
| Refrigerator | GE Refrigerator GTS18FBMFRWW, | |
| | S/N #FF759776 | |
| | Jordon Scientific Refrigerator | |
| Repeater Pipette | Eppendorf Repeater Plus, incl. CombiTips | |
| | Plus (10 mL, 5 mL, 2.5 mL) | |
| Rotisserie | Barnstead 415110, S/N #1105060242886, | |
| | incl. sample holder for | |
| | 15 mL conical tubes | |
| Tabletop | Eppendorf Centrifuge 5414D | |
| Centrifuge | (24-place fixed-angle rotor for 1.5- | |
| | 2.0 mL tubes/F-24-45-11) | |
| Thermogravimetric | TA Instruments, TGA Q500 | |
| Balance | | |
| UV-HPLC | Pump System: | Shimadzu Prominence LC-20 AT |
| System 12 | Auto-sampler: | Shimadzu Prominence SIL-20AC |
| | Degasser: | Shimadzu Prominence DGU-20As |
| | Detector: | Shimadzu Prominence SPD-20A |
| | Column Oven: | Shimadzu Prominence CTO-20A |
| | Software: | Shimadzu Class VP |
| | | Client/Server V7.4 |
| | Column: | MAC-MOD Halo C18, |
| | | Part #: 92814-602, |
| | | SN#: AH142237/USRG003098 |
| Vortex Mixer | National Labnet Co. Inc, VX100 (S01100) | |
| XRD Equipment | Diffractometer: | Rigaku MiniFlex, #2005G302, S/N |
| | | #CD04539 (30 kV/50 mA, Cu-Kα) |
| | Cooling unit: | Haskris WA1 |
| | Sample Holder | Six-Position Sample |
| | Mount: | Changer with Sample |
| | | Spinner ASC-6, #2455E431 |
| | Sample Holder: | Zero Background Sample |
| | | Holder-100 micron indent, |
| | | round, #SH-LBSI511-RNDB |

### Example 16. Long-Term Stability Studies

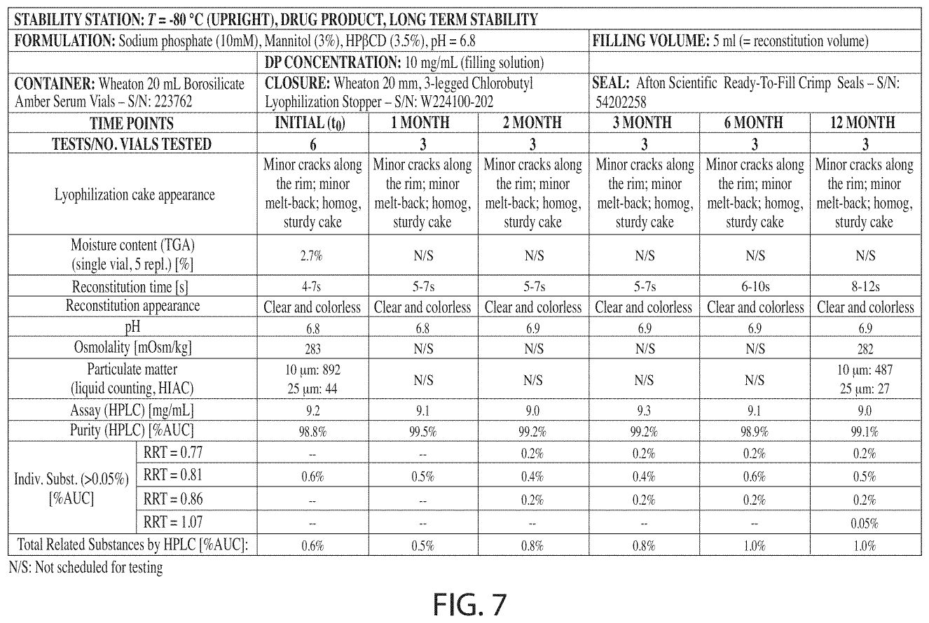

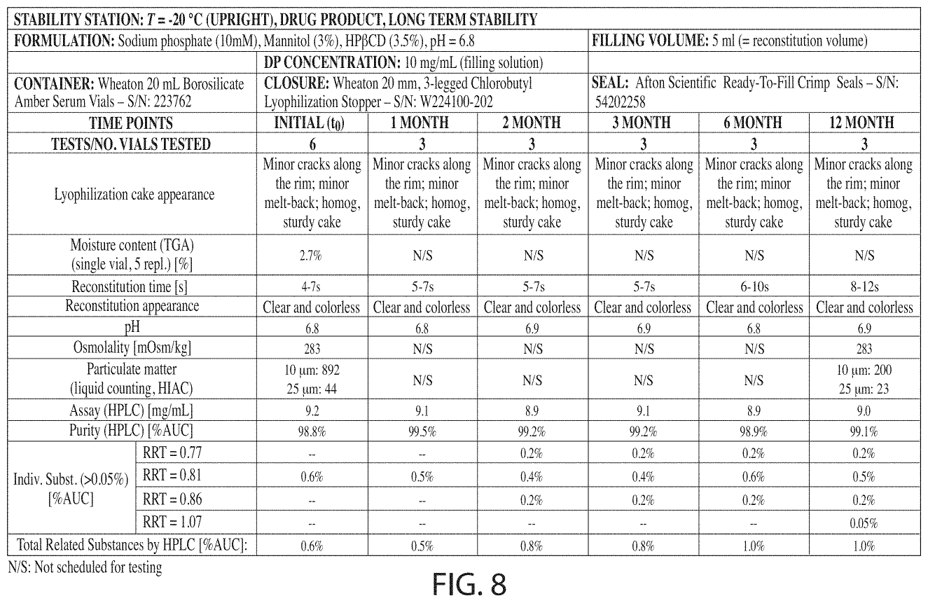

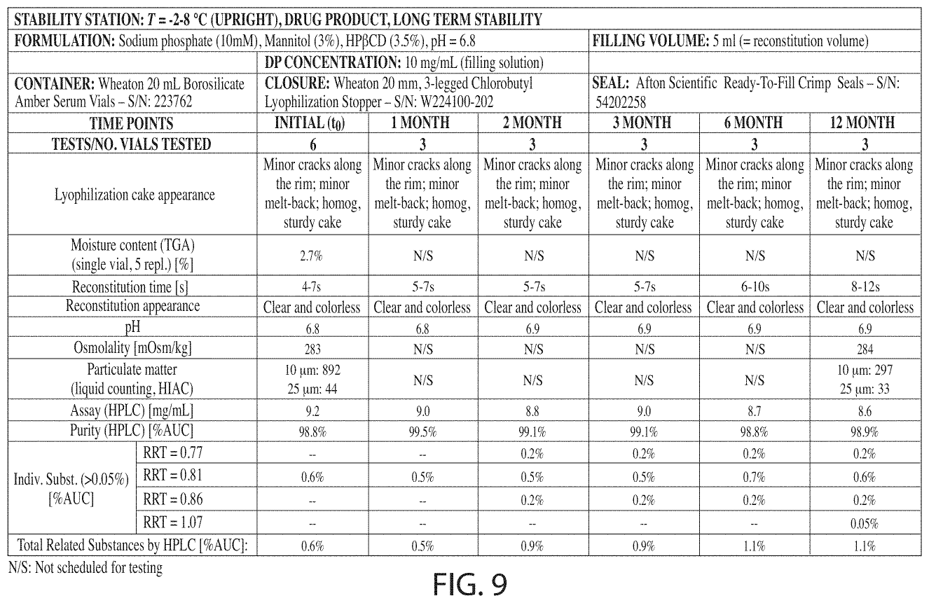

Exemplary long-term stability studies of the Compound 1 lyophilized drug product are shown in FIGS. 7-9 for T=−80° C., T=−20° C., and T=2-8° C., respectively. The studies were initiated by collecting data of an initial time point (to) and subjecting the required amount of sample to the respective testing conditions. Stability of the Compound 1 lyophilized drug product was evaluated after t=1 month, 3 month, 6 month, 9 month, and 12 month. The tests include appearance, reconstitution time, reconstitution appearance, recovery & impurity (HPLC assay), pH, and LPC (HIAC, particulates).

### Example 17. Compounding Process for Compound 1 Liquid Injectable Formulation

Compounding process for liquid formulations of Compound 1, designed for dilution into infusion vehicles was developed with 3.0 mg/mL of Compound 1 (or 3.13 mg/mL Compound 1 free base equivalent), phosphate buffer solution (PBS), and pH of 6.4-7.2. The compounding process was applied over a wide range of scales (25-500 mL).

The compounding of the drug product required a neutralization step using 0.5N sodium hydroxide solution and buffering with PBS to adjust the pH to values compatible with an IV infusion. The compounding process also required a filtration step, which also served as aseptic processing. Either a minimal loss or no loss of Compound 1 was observed.

The compounded formulations were diluted into infusion vehicles: normal saline, 5% dextrose in water (D5W), and lactated Ringer's solution (buffered and unbuffered). The dilution into normal saline was carried out at 100-fold and 600-fold dilution.

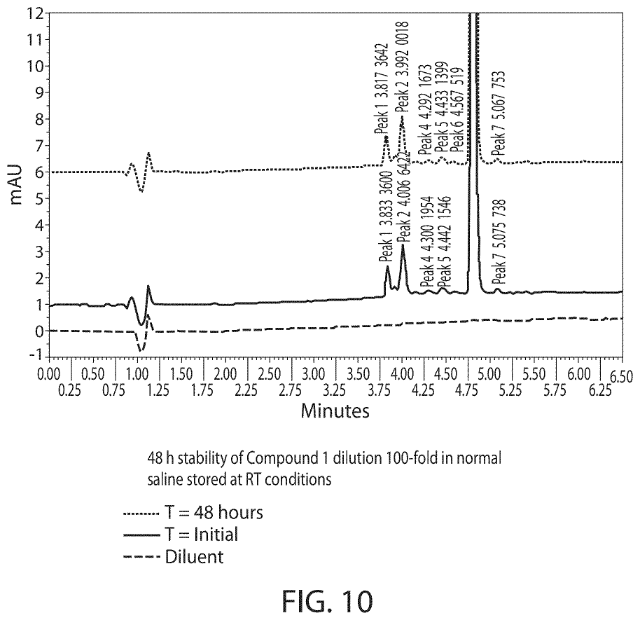

Compound 1 formulations were stable at 2-8° C. for at least one week with nominal degradation of <4%. The observed Compound 1 was within the error range of sample preparation, so it is feasible that no measurable degradation was occurring during the studied time frame. When Compound 1 formulation was diluted into normal saline, the resulting infusate solution at c(Compound 1)=0.03 mg/mL was stable at ambient temperature for at least 48 hours. The pH was constant over 48 hours and no significant loss in Compound 1 recovery was observed. The purity for Compound 1 appeared unchanged for over 48 hours and the chromatographic trace recorded after 48 hours did not reveal additional peaks or growth of observed degradants (FIG. 10).

### Example 18. Efficacy Study of Compound 1 in a Hound Cardiopulmonary Bypass Model

The objective of this study was to demonstrate the efficacy of Compound 1 compared to the Standard of Care (SOC), heparin, for preventing activation of blood coagulation components while using the Cardiopulmonary Bypass (CPB) circuit during an extended run time on Day 1 in a mixed breed hound dog model. The study design is shown in Table 19:

TABLE 19
Experimental Design (Target Doses of Compound 1^{b})

| | | | IV | | | IV | | | | | |
|---|---|---|---|---|---|---|---|---|---|---|---|
| | | IV | Bolus | | | Infusion | Dose | Dose | | | |
| | | Loading | Dose | Dose | | Dose | Infusion | Concentration | | Compound | |
| | No. of | Dose | Volume | Concentration | | Level | Rate | for IV | | 1 in Prime | |
| Group | Animals | (mg/kg) | (mL/kg) | for IV Bolus | | (mg/kg/hr) | (mL/kg/hr) | Infusion | | Solution | |
| 1 | 3 | NA | NA | NA | | 3 | 5 | 0.6 | mg/mL | NA | |
| 2 | 2 | 3 | 1 | 0.6 | μg/mL and | 3 | 5 | 0.6 | μg/mL | 0.01 | mg/mL |
| | | | | 3 | mg/mL^{a} | | | | | | |
| 3 | 2 | 10 | 1 | 10 | mg/mL | 10 | 5 | 2.0 | mg/mL | 0.01 | mg/mL |
| 4 | 2 | 10 | 1 | 10 | mg/mL | 10 | 5 | 2.0 | mg/mL | 0.01 | mg/mL |
| 5 | 2 | 10 | 1 | 10 | mg/mL | 10 | 5 | 2.0 | mg/mL | 0.01 | mg/mL |

NA—Not Applicable
^{a}Animal No. 1001 received 0.6 μg/mL and Animal No. 1004 received 3 mg/mL.
^{b}Doses shown are targets for the dosing on this study; actual dose values are shown in the results section.

The following parameters and endpoints were evaluated in this study: mortality, body weight, physical, clinical pathology parameters (hematology and coagulation), coagulation time, and bioanalytical parameters.

### Experimental Design

### Administration

The vehicle and test article were administered via intravenous (IV) infusion once on Day 1 for 135 minutes (initiated 30 minutes prior to starting the Cardiopulmonary Bypass (CPB) and continuing for 105 minutes of CPB). Group 2 animals received a 0.6 μg/mL or 3.0 mg/mL IV bolus dose immediately prior to the start of IV infusion. Group 3, 4, and 5 animals received a 10 mg/kg IV bolus dose prior to the start of the IV infusion; with the CPB machine primed with test article at 10 μg/mL.

### Surgical Procedure

Group 1 had an infusion pump setup with an open system/reservoir. Infusion of the Compound 1 was started 30 minutes prior to the animal being placed on the CPB pump. The CPB pump was primed with 0.9% saline.

Groups 2, 3, and 4 had an infusion pump setup with an open system/reservoir. Venous and arterial sheaths were flushed with the Compound 1 at a concentration of 10 μg/mL. An IV bolus dose of the test article was administered immediately prior to the start of the infusion. Infusion of Compound 1 was started 30 minutes prior to the animal being placed on the CPB pump. The CPB patient was primed with 10 μg/mL of the Compound 1 prior to initiation of the CPB pump.

Group 5 had an infusion pump setup with a closed system/“bag.” Venous and arterial sheaths were then flushed with Compound 1 at 10 μg/mL. An IV bolus dose of the Compound 1 was administered immediately prior to the start of the infusion. Infusion of Compound 1 was started 30 minutes prior to the animal being placed on the CPB pump.

### Results

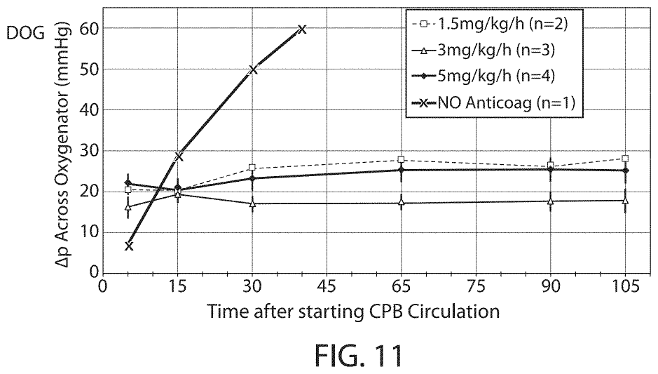

FIG. 11 shows pressure gradients assessed across the membrane oxygenator. Studies previously conducted with no anticoagulant demonstrated that the pressure across the membrane oxygenator built within 15 minutes of pump start and exponentially increased over the next 30 minutes such that the oxygenator was occluded and the circulation was stopped, whereas with Compound 1 at multiple doses, the pressure gradient across the membrane oxygenator stayed consistent through the entire run, indicating that the test article successfully maintained anticoagulation allowing the continuation of the pump run for the entirety of the protocol.

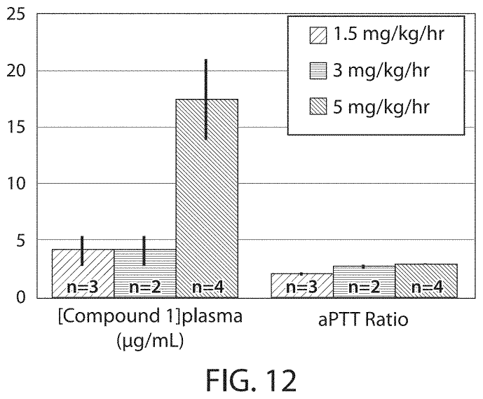

FIG. 12 shows a correlation between Compound 1 plasma concentration and aPTT. All animals survived to study termination. Overall, Compound 1 was not associated with any increases in morbidity or mortality at the dose levels used in this study during the Cardiopulmonary bypass/ECMO protocol.

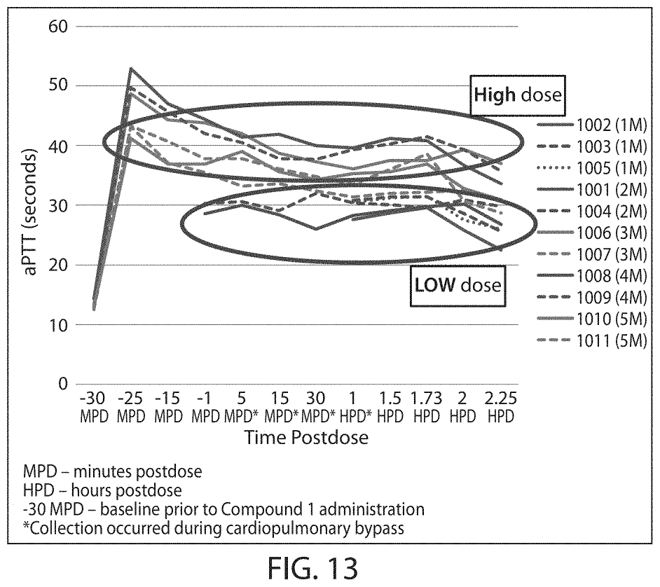

During Compound 1 infusion and prior to CPB, aPTT was moderately to markedly prolonged in all animals (FIG. 13). Prolongations in aPTT persisted throughout Compound 1 infusion and CPB. In groups that received a loading dose of Compound 1 (Groups 2 through 5), prolongations in aPTT were most pronounced prior to (Group 3 through 5) or during the first 30 minutes of CPB (Group 2), but then improved slightly before reaching steady-state. Group 1 animals did not receive an Compound 1 loading dose, and prolongations in aPTT remained relatively consistent at all measured timepoints during Compound 1 infusion in this group. In all groups following cessation of Compound 1 infusion and CPB, aPTT trended towards baseline values, but remained moderately prolonged at the conclusion of the study.

### CONCLUSIONS

Administration of the Compound 1 to the model was successful in preventing the activation of blood coagulation in components of cardiopulmonary bypass. The anticoagulant effects of Compound 1 were selective to inhibition of activated partial thromboplastin time (aPTT). Additionally, the data demonstrated that adding a bolus dose immediately prior to starting the infusion enabled targeted plasma levels of Compound 1 to rapidly be achieved, along with desired steady state levels, and was sufficient to achieve a successful 105-minute CPB run and prevent coagulation in most of the circuit components.

Overall, these data indicate that Compound 1 may be an acceptable alternative to heparin in preventing blood coagulation in components of cardiopulmonary bypass.

What is claimed is:

1. An aqueous pharmaceutical composition comprising a compound of Formula (I-A) 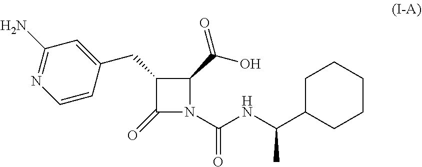 [SMILES: C[C@@H](NC(=O)N1C(=O)[C@H](Cc2ccnc(N)c2)[C@H]1C(=O)O)C1CCCCC1]
or a pharmaceutically acceptable salt thereof, a cyclodextrin, and an excipient.

2. The pharmaceutical composition of claim 1, wherein the cyclodextrin is selected from the group consisting of alkyl cyclodextrin, hydroxyalkyl cyclodextrin, carboxyalkyl cyclodextrin, and sulfoalkyl ether cyclodextrin.

3. The pharmaceutical composition of claim 1 or 2, wherein the cyclodextrin is hydroxypropyl β-cyclodextrin.

4. The pharmaceutical composition of claim 1 or 2, wherein the cyclodextrin is sulfobutyl ether β-cyclodextrin.

5. The pharmaceutical composition of any one of claims 1 to 4, wherein the excipient is a sugar (e.g., a saccharide (e.g., monosaccharide, disaccharide, or polysaccharide)) or a sugar alcohol.

6. The pharmaceutical composition of any one of claims 1 to 5, wherein the excipient is sucrose, lactose, trehalose, dextran, erythritol, arabitol, xylitol, sorbitol, or mannitol, or a combination thereof.

7. The pharmaceutical composition of any one of claims 1 to 6, wherein the excipient is mannitol.

8. The pharmaceutical composition of any one of claims 1 to 6, wherein the excipient is lactose.

9. The pharmaceutical composition of any one of claims 1 to 8, further comprising a buffer.

10. The pharmaceutical composition of claim 9, wherein the buffer is a monoprotic acid or a polyprotic acid or a combination thereof.

11. The pharmaceutical composition of claim 9 or 10, wherein the buffer is a solution of one or more substances.

12. The pharmaceutical composition of any one of claims 9 to 11, wherein the buffer is a solution of a salt of a weak acid and a weak base.

13. The pharmaceutical composition of any one of claims 9 to 11, wherein the buffer is a solution of a salt of the weak acid with a strong base.

14. The pharmaceutical composition of any one of claims 9 to 13, wherein the buffer is selected from the group consisting of a maleate buffer, a citrate buffer, and a phosphate buffer.

15. The pharmaceutical composition of any one of claims 9 to 14, wherein the buffer is a phosphate buffer.

16. The pharmaceutical composition of claim 15, wherein the phosphate buffer is a solution of monosodium phosphate, disodium phosphate, trisodium phosphate, or a combination thereof.

17. The pharmaceutical composition of any one of claims 1 to 17, further comprising a solubilizing agent.

18. The pharmaceutical composition of claim 17, wherein the solubilizing agent is a polyoxyethylene sorbitan ester (e.g, TWEEN® 20) or a polyethylene glycol (e.g., PEG400).

19. The pharmaceutical composition of any one of claims 1 to 18, wherein the pH of the composition is from about 2 to about 8.

20. The pharmaceutical composition of any one of claims 1 to 19, wherein the pH of the composition is about 6.8.

21. The pharmaceutical composition of any one of claims 1 to 20, wherein the concentration of the compound of Formula (I-A) is from about 0.1 mg/mL to about 100 mg/mL.

22. The pharmaceutical composition of any one of claims 1 to 21, wherein the concentration of the compound of Formula (I-A) is about 10 mg/mL.

23. The pharmaceutical composition of any one of claims 9 to 22, wherein the concentration of the buffer is from about 1 mM to about 500 mM.

24. The pharmaceutical composition of any one of claims 9 to 23, wherein the concentration of the buffer is about 10 mM.

25. The pharmaceutical composition of claim 23 or 24, wherein the buffer is a phosphate buffer.

26. The pharmaceutical composition of any one of claims 1 to 25, wherein the cyclodextrin is in an amount of from about 0.1% to about 10% (e.g., about 0.5% to about 6% (e.g., about 0.7% to about 5.6% (e.g., about 2.1% to about 5%))) by weight relative to weight of the compound of Formula (I-A).

27. The pharmaceutical composition of any one of claims 1 to 26, wherein the cyclodextrin is in an amount of about 3.5% by weight relative to weight of the compound of Formula (I-A).

28. The pharmaceutical composition of any one of claims 1 to 26, wherein the cyclodextrin is in an amount of about 5% by weight relative to weight of the compound of Formula (I-A).

29. The pharmaceutical composition of any one of claims 26 to 28, wherein the cyclodextrin is hydroxypropyl β-cyclodextrin.

30. The pharmaceutical composition of any one of claims 1 to 29, wherein the excipient is in an amount of from about 0.1% to about 10% by weight relative to weight of the compound of Formula (I-A).

31. The pharmaceutical composition of any one of claims 1 to 30, wherein the excipient is in an amount of about 3% by weight relative to weight of the compound of Formula (I-A).

32. The pharmaceutical composition of any one of claims 1 to 30, wherein the excipient is in an amount of about 5% by weight relative to weight of the compound of Formula (I-A).

33. The pharmaceutical composition of any one of claims 30 to 32, wherein the excipient is mannitol.

34. The pharmaceutical composition of any one of claims 30 to 32, wherein the excipient is lactose.

35. A pharmaceutical composition comprising particles, wherein the particles comprise a compound of Formula (I-A) 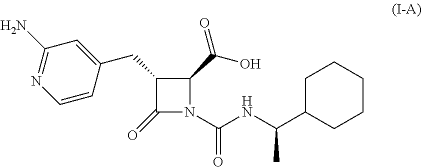 [SMILES: C[C@@H](NC(=O)N1C(=O)[C@H](Cc2ccnc(N)c2)[C@H]1C(=O)O)C1CCCCC1]
or a pharmaceutically acceptable salt thereof, a cyclodextrin, and a bulking agent.

36. The pharmaceutical composition of claim 35, wherein the cyclodextrin is selected from the group consisting of alkyl cyclodextrin, hydroxyalkyl cyclodextrin, carboxyalkyl cyclodextrin, and sulfoalkyl ether cyclodextrin.

37. The pharmaceutical composition of claim 35 or 36, wherein the cyclodextrin is hydroxypropyl β-cyclodextrin.

38. The pharmaceutical composition of claim 35 or 36, wherein the cyclodextrin is sulfobutyl ether β-cyclodextrin.

39. The pharmaceutical composition of any one of claims 35 to 38, wherein the bulking agent is a sugar (e.g., a saccharide (e.g., monosaccharide, disaccharide, or polysaccharide)) or a sugar alcohol.

40. The pharmaceutical composition of any one of claims 35 to 39, wherein the bulking agent is sucrose, lactose, trehalose, dextran, erythritol, arabitol, xylitol, sorbitol, or mannitol, or a combination thereof.

41. The pharmaceutical composition of any one of claims 35 to 40, wherein the bulking agent is mannitol.

42. The pharmaceutical composition of any one of claims 35 to 40, wherein the bulking agent is lactose.

43. The pharmaceutical composition of any one of claims 35 to 42, wherein the bulking agent is a lyoprotectant.

44. The pharmaceutical composition of any one of claims 35 to 43 wherein the concentration of the compound of Formula (I-A) is from about 0.1 to about 10% by weight of the composition.

45. The pharmaceutical composition of any one of claims 35 to 44, wherein the concentration of the compound of Formula (I-A) is about 1% by weight of the composition.

46. The pharmaceutical composition of any one of claims 35 to 44, wherein the concentration of the compound of Formula (I-A) is about 0.3% by weight of the composition.

47. The pharmaceutical composition of any one of claims 35 to 46, wherein the cyclodextrin is in an amount of from about 0.1% to about 10% (e.g., about 0.5% to about 6% (e.g., about 0.7% to about 5.6% (e.g., about 2.1% to about 5%))) by weight relative to weight of the compound of Formula (I-A).

48. The pharmaceutical composition of any one of claims 35 to 47, wherein the cyclodextrin is in an amount of about 3.5% by weight relative to weight of the compound of Formula (I-A).

49. The pharmaceutical composition of any one of claims 35 to 47, wherein the cyclodextrin is in an amount of about 5% by weight relative to weight of the compound of Formula (I-A).

50. The pharmaceutical composition of any one of claims 47 to 49, wherein the cyclodextrin is hydroxypropyl β-cyclodextrin.

51. The pharmaceutical composition of any one of claims 35 to 50, wherein the bulking agent is in an amount of from about 0.1% to about 10% by weight relative to weight of the compound of Formula (I-A).

52. The pharmaceutical composition of any one of claims 35 to 51, wherein the bulking agent is in an amount of about 3% by weight relative to weight of the compound of Formula (I-A).

53. The pharmaceutical composition of any one of claims 35 to 51, wherein the bulking agent is in an amount of about 5% by weight relative to weight of the compound of Formula (I-A).

54. The pharmaceutical composition of any one of claims 51 to 53, wherein the bulking agent is mannitol.

55. The pharmaceutical composition of any one of claims 51 to 53, wherein the bulking agent is lactose.

56. A process for preparing an aqueous pharmaceutical composition from the pharmaceutical composition of any one of claims 35 to 55, the process comprising reconstituting the pharmaceutical composition into an aqueous medium, thereby forming the aqueous composition.

57. The process of claim 56, wherein the aqueous medium is deionized water.

58. The process of claim 56 or 57, wherein the aqueous medium comprises sodium chloride.

59. The process of claim 56 or 57, wherein the aqueous medium comprises about 5% dextrose.

60. The process of any one of claims 56 to 59, wherein the composition is prepared to be suitable for parenteral administration to a subject in need thereof.

61. The process of any one of claims 56 to 59, wherein the composition is prepared to be suitable for intramuscular, subcutaneous or intravenous administration to a subject in need thereof.

62. A method of treating a thromboembolic disorder in a subject in need thereof, the method comprising administering to the subject an effective amount of a pharmaceutical composition of any one of claims 1-34, wherein the blood of the subject is contacted with an artificial surface.

63. A method of reducing the risk of a thromboembolic disorder in a subject in need thereof, the method comprising administering to the subject an effective amount of a pharmaceutical composition of any one of claims 1-34, wherein the blood of the subject is contacted with an artificial surface.

64. A method of prophylaxis of a thromboembolic disorder in a subject in need thereof, the method comprising administering to the subject an effective amount of a pharmaceutical composition of any one of claims 1-34, wherein the blood of the subject is contacted with an artificial surface.

65. The method of any one of claims 62-64, wherein the artificial surface is in contact with blood in the subject's circulatory system.

66. The method of any one of claims 62-65, wherein the artificial surface is an implantable device, a dialysis catheter, a cardiopulmonary bypass circuit, an artificial heart valve, a ventricular assist device, a small caliber graft, a central venous catheter, or an extracorporeal membrane oxygenation (ECMO) apparatus.

67. The method of any one of claims 62-66, wherein the artificial surface causes or is associated with the thromboembolic disorder.

68. The method of any one of claims 62-67, wherein the thromboembolic disorder is a venous thromboembolism, deep vein thrombosis, or pulmonary embolism.

69. The method of any one of claims 62-67, wherein the thromboembolic disorder is a blood clot.

70. The method of any one of claims 62-69, further comprising conditioning the artificial surface with a separate dose of a pharmaceutical composition of any one of claims 1-34 prior to contacting the artificial surface with blood in the circulatory system of the subject.

71. The method of any one of claims 62-69, further comprising conditioning the artificial surface with a separate dose of a pharmaceutical composition of any one of claims 1-34 prior to or during administration of the pharmaceutical composition to the subject.

72. The method of any one of claims 62-69, further comprising conditioning the artificial surface with a separate dose of a pharmaceutical composition of any one of claims 1-34 prior to and during administration of the pharmaceutical composition to the subject.

73. The method of any one of claims 62-72, wherein the artificial surface is a cardiopulmonary bypass circuit.

74. The method of any one of claims 62-72, wherein the artificial surface is an extracorporeal membrane oxygenation (ECMO) apparatus.

75. The method of claim 74, wherein the ECMO apparatus is venovenous ECMO apparatus or venoarterial ECMO apparatus.

76. A method of preventing or reducing a risk of a thromboembolic disorder in a subject during or after a medical procedure, comprising:
(i) administering to the subject an effective amount of a pharmaceutical composition of any one of claims 1-34, before, during, or after the medical procedure; and
(ii) contacting blood of the subject with an artificial surface;
thereby preventing or reducing the risk of the thromboembolic disorder during or after the medical procedure.

77. The method of claim 76, wherein the artificial surface is conditioned with a pharmaceutical composition of any one of claims 1-34 prior to administration of the pharmaceutical composition to the subject prior to, during, or after the medical procedure.

78. The method of claim 77, wherein the pharmaceutical composition for conditioning the artificial surface further comprises a solution, wherein the solution is selected from the group consisting of a saline solution, Ringer's solution, and blood.

79. The method of any one of claims 76-78, wherein the thromboembolic disorder is a blood clot.

80. The method of any one of claims 76-79, wherein the medical procedure comprises one or more of i) a cardiopulmonary bypass, ii) oxygenation and pumping of blood via extracorporeal membrane oxygenation, iii) assisted pumping of blood (internal or external), iv) dialysis of blood, v) extracorporeal filtration of blood, vi) collection of blood from the subject in a repository for later use in an animal or a human subject, vii) use of venous or arterial intraluminal catheter(s), viii) use of device(s) for diagnostic or interventional cardiac catherisation, ix) use of intravascular device(s), x) use of artificial heart valve(s), and xi) use of artificial graft(s).

81. The method of any one of claims 76-80, wherein the medical procedure comprises a cardiopulmonary bypass.

82. The method of any one of claims 76-80, wherein the medical procedure comprises an oxygenation and pumping of blood via extracorporeal membrane oxygenation (ECMO).

83. The method of claim 82, wherein the ECMO is venovenous ECMO or venoarterial ECMO.

84. The method of any one of claims 62-83, wherein the subject is in contact with the artificial surface for at least 1 day (e.g., about 2 days, about 3 days, about 4 days, about 5 days, about 6 days, about 1 week, about 10 days, about 2 weeks, about 3 weeks, about 4 weeks, about 2 months, about 3 months, about 6 months, about 9 months, about 1 year).

85. A method of treating the blood of a subject in need thereof, the method comprising administering to the subject an effective amount of a pharmaceutical composition of any one of claims 1-34.

86. The method of any one of claims 62-85, wherein the pharmaceutical composition is administered to the subject intravenously.

87. The method of any one of claims 62-85, wherein the pharmaceutical composition is administered to the subject subcutaneously.

88. The method of any one of claims 62-85, wherein the pharmaceutical composition is administered to the subject as a continuous intravenous infusion.

89. The method of any one of claims 62-85, wherein the pharmaceutical composition is administered to the subject as a bolus.

90. The method of any one of claims 62-89, wherein the subject is a human.

91. The method of any one of claims 62-90, wherein the subject has an elevated risk of a thromboembolic disorder.

92. The method of claim 91, wherein the thromboembolic disorder is a result of a complication in surgery.

93. The method of any one of claims 62-92, wherein the subject is sensitive to or has developed sensitivity to heparin.

94. The method of any one of claims 62-92, wherein the subject is resistant to or has developed resistance to heparin.

95. The method of any one of claims 62-94, wherein the subject is a pediatric subject.

96. The method of any one of claims 62-94, wherein the subject is an adult.
